SEQUENCE LISTING (25 sequences)

SEQ ID NO: 1 - protein, 13 aa, Artificial Sequence
  Synthetic Construct
GCCSDPRCRYRCR

SEQ ID NO: 2 - protein, 13 aa, Artificial Sequence
  Synthetic Construct
  MISC_FEATURE (9)..(9): Citrulline
  MISC_FEATURE (10)..(10): 3-Iodo-tyrosine
GCCTDPRCXXQCY

SEQ ID NO: 3 - protein, 15 aa, Artificial Sequence
  Synthetic Construct
  MISC_FEATURE (1)..(2): Any Amino acid other than Cysteine
  MISC_FEATURE (5)..(5): Any Amino acid other than Cysteine
  MISC_FEATURE (10)..(12): Any Amino acid other than Cysteine
  MISC_FEATURE (14)..(15): Any Amino acid other than Cysteine
XXCCXDPRCXXXCXX

SEQ ID NO: 4 - protein, 15 aa, Artificial Sequence
  Synthetic Construct
  MISC_FEATURE (1)..(1): E or D
  MISC_FEATURE (2)..(2): Any Amino acid other than Cysteine
  MISC_FEATURE (5)..(5): Any Amino acid other than Cysteine
  MISC_FEATURE (10)..(12): Any Amino acid other than Cysteine
  MISC_FEATURE (14)..(14): Any Amino acid other than Cysteine
  MISC_FEATURE (15)..(15): K or Dap
XXCCXDPRCXXXCXX

SEQ ID NO: 5 - protein, 15 aa, Artificial Sequence
  Synthetic Construct
  MISC_FEATURE (1)..(1): E or D
  MISC_FEATURE (2)..(2): Any Amino acid other than Cysteine
  MISC_FEATURE (10)..(11): Any Amino acid other than Cysteine
  MISC_FEATURE (15)..(15): K or Dap
XXCCTDPRCXXQCYX

SEQ ID NO: 6 - protein, 15 aa, Artificial Sequence
  Synthetic Construct
  MISC_FEATURE (1)..(1): E or D
  MISC_FEATURE (2)..(2): G or bA
  MISC_FEATURE (10)..(10): R or (Cit)
  MISC_FEATURE (11)..(11): Y or (iY)
  MISC_FEATURE (15)..(15): K or Dap
XXCCTDPRCXXQCYX

SEQ ID NO: 7 - protein, 15 aa, Artificial Sequence
  Synthetic Construct
  MISC_FEATURE (10)..(10): Citrulline
  MISC_FEATURE (15)..(15): L-2,3-diaminopropionic acid
DGCCTDPRCXYQCYX

SEQ ID NO: 8 - protein, 15 aa, Artificial Sequence
  Synthetic Construct
  MISC_FEATURE (10)..(10): Citrulline
DGCCTDPRCXYQCYK

SEQ ID NO: 9 - protein, 15 aa, Artificial Sequence
  Synthetic Construct
  MISC_FEATURE (10)..(10): Citrulline
EGCCTDPRCXYQCYK

SEQ ID NO: 10 - protein, 15 aa, Artificial Sequence
  Synthetic Construct
  MISC_FEATURE (2)..(2): beta-alanine
  MISC_FEATURE (10)..(10): Citrulline
EXCCTDPRCXYQCYK

SEQ ID NO: 11 - protein, 15 aa, Artificial Sequence
  Synthetic Construct
  MISC_FEATURE (10)..(10): Citrulline
  MISC_FEATURE (11)..(11): 3-Iodo-tyrosine
EGCCTDPRCXXQCYK

SEQ ID NO: 12 - protein, 15 aa, Artificial Sequence
  Synthetic Construct
  MISC_FEATURE (11)..(11): 3-Iodo-tyrosine
EGCCTDPRCRXQCYK

SEQ ID NO: 13 - protein, 13 aa, Artificial Sequence
  Synthetic Construct
  MISC_FEATURE (1)..(1): Any Amino acid other than Cysteine
  MISC_FEATURE (4)..(4): Any Amino acid other than Cysteine
  MISC_FEATURE (9)..(11): Any Amino acid other than Cysteine
  MISC_FEATURE (13)..(13): Any Amino acid other than Cysteine
XCCXDPRCXXXCX

SEQ ID NO: 14 - protein, 13 aa, Artificial Sequence
  Synthetic Construct
  MISC_FEATURE (1)..(1): Any Amino acid other than Cysteine
  MISC_FEATURE (4)..(4): Any Amino acid other than Cysteine
  MISC_FEATURE (9)..(9): (Cit) or positive amino acid (His, Arg, or Lys)
  MISC_FEATURE (10)..(10): aromatic amino acid (Phe, Tyr, or Trp)
  MISC_FEATURE (11)..(11): positive amino acid (His, Arg, or Lys)
  MISC_FEATURE (13)..(13): aromatic amino acid (Phe, Tyr, or Trp)
XCCXDPRCXXXCX

SEQ ID NO: 15 - protein, 13 aa, Artificial Sequence
  Synthetic Construct
  MISC_FEATURE (9)..(10): Any Amino acid other than Cysteine
  MISC_FEATURE (13)..(13): Any Amino acid other than Cysteine
GCCTDPRCXXQCX

SEQ ID NO: 16 - protein, 13 aa, Artificial Sequence
  Synthetic Construct
  MISC_FEATURE (9)..(9): Citrulline or Arg
  MISC_FEATURE (10)..(10): iY or Y
  MISC_FEATURE (13)..(13): bhY, Y, or bA
GCCTDPRCXXQCX

SEQ ID NO: 17 - protein, 13 aa, Artificial Sequence
  Synthetic Construct
  MISC_FEATURE (9)..(9): Citrulline
  MISC_FEATURE (10)..(10): 3-Iodo-tyrosine
GCCTDPRCXXQCY

SEQ ID NO: 18 - protein, 13 aa, Artificial Sequence
  Synthetic Construct
  MISC_FEATURE (9)..(9): Citrulline
  MISC_FEATURE (10)..(10): 3-Iodo-tyrosine
GCCTDPRCXXQCY

SEQ ID NO: 19 - protein, 13 aa, Artificial Sequence
  Synthetic Construct
  MISC_FEATURE (9)..(9): Citrulline
  MISC_FEATURE (10)..(10): 3-Iodo-tyrosine
GCCTDPRCXXQCY

SEQ ID NO: 20 - protein, 14 aa, Artificial Sequence
  Synthetic Construct
  MISC_FEATURE (1)..(1): Any Amino acid other than Cysteine
  MISC_FEATURE (4)..(4): Any Amino acid other than Cysteine
  MISC_FEATURE (9)..(11): Any Amino acid other than Cysteine
  MISC_FEATURE (13)..(14): Any Amino acid other than Cysteine
XCCXDPRCXXXCXX

SEQ ID NO: 21 - protein, 14 aa, Artificial Sequence
  Synthetic Construct
  MISC_FEATURE (1)..(1): Any Amino acid other than Cysteine
  MISC_FEATURE (4)..(4): Any Amino acid other than Cysteine
  MISC_FEATURE (9)..(9): (Cit) or positive amino acid (His, Arg, or Lys)
  MISC_FEATURE (10)..(10): aromatic amino acid (Phe, Tyr, or Trp)
  MISC_FEATURE (11)..(11): positive amino acid (His, Arg, or Lys)
  MISC_FEATURE (13)..(13): aromatic amino acid (Phe, Tyr, or Trp)
  MISC_FEATURE (14)..(14): Any Amino acid other than Cysteine
XCCXDPRCXXXCXX

SEQ ID NO: 22 - protein, 14 aa, Artificial Sequence
  Synthetic Construct
  MISC_FEATURE (9)..(10): Any Amino acid other than Cysteine
  MISC_FEATURE (13)..(14): Any Amino acid other than Cysteine
GCCTDPRCXXQCXX

SEQ ID NO: 23 - protein, 14 aa, Artificial Sequence
  Synthetic Construct
  MISC_FEATURE (9)..(9): Citrulline or Arg
  MISC_FEATURE (10)..(10): iY or Y
  MISC_FEATURE (13)..(13): bhY, Y, or bA
GCCTDPRCXXQCXR

SEQ ID NO: 24 - protein, 14 aa, Artificial Sequence
  Synthetic Construct
  MISC_FEATURE (10)..(10): 3-Iodo-tyrosine
  MISC_FEATURE (13)..(13): L-beta-homo-tyrosine
GCCTDPRCRXQCXR

SEQ ID NO: 25 - protein, 14 aa, Artificial Sequence
  Synthetic Construct
  MISC_FEATURE (10)..(10): 3-Iodo-tyrosine
  MISC_FEATURE (13)..(13): beta-alanine
GCCTDPRCRXQCXR

(19) United States
(12) Patent Application Publication
(10) Pub. No.: US 2023/0391838 A1
(43) Pub. Date: Dec. 7, 2023

(54) Conformationally-Constrained Alpha-RGIA Analogues

(71) Applicant: University of Utah Research Foundation, Salt Lake City, UT (US)

(72) Inventors: Joseph Michael McIntosh, Salt Lake City, UT (US); Nan Zheng, Salt Lake City, UT (US); Hung-Chieh Chou, Salt Lake City, UT (US)

(21) Appl. No.: 17/760,668

(22) PCT Filed: Jun. 3, 2021
(86) PCT No.: PCT/US2021/035799

§ 371 Date: Mar. 15, 2022

Related U.S. Application Data
(60) Provisional application No. 63/034,395, filed on Jun. 3, 2020.

(51) Int. Cl. C07K 14/435

(52) U.S. Cl. CPC C07K 14/43504

(57) ABSTRACT

α-RgIA4 peptide analogs, compositions therewith, and methods for their treatment and use are disclosed and described. For example, an α-RgIA4 peptide analog can comprise a recognition finger region configured to bind to an α9α10 nicotinic acetylcholine receptor, and a side chain bonding configuration that protects an inter-cysteine sulfur linkage.

### RELATED APPLICATION

This application claims the benefit of U.S. Provisional Patent Application Ser. No. 63/034,395, filed Jun. 3, 2020, the entirety of which is incorporated herein by reference.

### GOVERNMENT INTEREST

This invention was made with government support under grant numbers GM048677, GM103801, GM136430 awarded by the National Institutes of Health and grant number W81WXH-17-1-0413 awarded by the Office of the Assistant Secretary of Defense for Health Affairs. The Government has certain rights to this invention.

### FIELD OF THE DISCLOSURE

The present disclosure relates to peptides and their analogs and therapeutic uses therefor. Accordingly, the present disclosure relates generally to the fields of biology, cell physiology, chemistry, pharmaceutical sciences, medicine, and other health sciences.

### BACKGROUND

Neuropathic pain is debilitating both physically and psychologically and is a highly prevalent complication of a large variety of diseases, including cancer, diabetes, stroke, AIDS and nerve damage. Opioids have been a first line of defense in treating such pain. However, the use of opioid-based medications for the treatment of neuropathic pain is challenging not only because of severe side-effects, but also due to the strong propensity for drug tolerance and addiction in long-term use. As a result, non-opioid therapeutics for the treatment of neuropathic pain continue to be sought.

Peptides from cone snail venoms have served as invaluable molecules for a variety of therapeutic uses, including to target pain-related receptors, including nicotinic acetylcholine receptors (nAChR). Unfortunately, native or wildtype cone snail peptides, such as α-RgIA can suffer from unfavorable physicochemical properties, which limit their therapeutic potential and require alteration in order to have therapeutic effectiveness in mammals. Furthermore, the process of altering native peptides into analogs, such as α-RgIA4 analogs, has many uncertainties and such analogs often achieve only mild potency. Accordingly, peptide analogs from cone snail venoms which have high potency in treating various conditions in mammals continue to be sought.

### SUMMARY

In one embodiment, an α-RgIA4 peptide analog can include a recognition finger region configured to bind to an α9α10 nicotinic acetylcholine receptor, and a side chain bonding configuration that protects an inter-cysteine sulfur linkage. The analog can have a binding affinity for the α9α10 nicotinic acetylcholine receptor that is at least 2.5% of a binding affinity of an α-RgIA4 peptide.

In another embodiment, an α-RgIA4 peptide analog can have a structure (e.g., a globular structure) maintained by a protected inter-cysteine sulfur linkage. The globular structure can provide a binding affinity for the α9α10 nicotinic acetylcholine receptor that is at least 2.5% of a binding affinity of an α-RgIA4 peptide.

In yet another embodiment, an α-RgIA4 peptide analog can include a recognition finger region comprising D P R; and cystine residues comprising C^{I}, C^{II}, C^{III}, and C^{IV}, The cysteine residues C^{I} and C^{III} can be linked by a first inter-cysteine sulfur linkage, and the cysteine residues C^{II} and C^{IV} can be linked by a second inter-cysteine sulfur linkage. The second inter-cysteine sulfur linkage can be protected by a side chain bonding configuration.

In another embodiment, a method of maintaining an α-RgIA4 potency for an α9α10 nicotinic acetylcholine receptor in an α-RgIA4 analog can include protecting inter-cysteine sulfur linkages with a side chain bonding configuration that maintains a recognition finger region of the analog in an α-RgIA4 configuration (e.g., a globular α-RgIA4 configuration).

In one more embodiment, a composition can include a combination of a therapeutically effective amount of the analog with a pharmaceutically acceptable carrier. In another embodiment, a method for treating in a subject, a condition that is responsive to α9α10 nicotinic acetylcholine receptor binding can include administering a therapeutically effective amount of the composition to the subject.

There has thus been outlined, rather broadly, the more important features of the disclosure so that the detailed description thereof that follows may be better understood, and so that the present contribution to the art may be better appreciated. Other features of the present disclosure will become clearer from the following detailed description, taken with the accompanying drawings and claims, or may be learned by the practice of the disclosure.

### BRIEF DESCRIPTION OF THE DRAWINGS

Features and advantages of the disclosure will be apparent from the detailed description which follows, taken in conjunction with the accompanying drawings, which together illustrate, by way of example, features of the disclosure.

FIG. 1a-A shows rapid conformation equilibrium between A1 (active) and A2 (inactive), and FIG. 1a-B shows constrained conformation disfavors conformation change from B1 (active) to B2 (inactive) in accordance with an example.

FIG. 1b shows an active conformation including a lactam bridge in accordance with an example.

FIG. 1c shows an active conformation including methylene thioacetal in accordance with an example.

FIG. 2 shows a synthetic route for macrocyclic α-RgIA analogues. a) Fmoc-SPPS. b) Boc₂O, DIEA, DCM. c) Method 1 for normal tyrosine (PG₁=allyl, PG₂=aloc): Pd(PPh₃)₄, DMBA, DCM; Method 2 for 3-iodo-tyrosine (PG₁=Dmab, PG₂=ivDde): 5% Hydrazine in DMF. d) PyBOP, DIEA, HOBt, DMF. e) TFA:H₂O:TIPS:EDT=95:2:2:1 (v/v) then RP-HPLC. f) 0.02 M Na₂HPO₄ (pH 8.0), air. g) 12, AcOH, H₂O in accordance with an example.

FIG. 3a shows A) Concentration responses of synthesized analogues on human α9α10 nAChRs. Data represents separate oocytes (n=3-6) measurement and error bar represents SD. B) Analogue 6 prevents chemotherapy-induced neuropathic pain on cold-plate testing. Dose=80 μg/kg (s.c.). Values are expressed as the mean±SEM (n=8-9) for each experimental determination. **p<0.01. Sal=saline; Ox=oxaliplatin; SD=standard deviation in accordance with an example.

FIG. 3b shows dose-responsive curves of Analogue 6 on human α9α10 versus α7 receptors in accordance with an example.

FIG. 3c shows LC-Chromatogram and MS-Spectrum for α-RgIA4 in accordance with an example.

FIG. 3d shows LC-Chromatogram and MS-Spectrum for analogue 1 in accordance with an example.

FIG. 3e shows LC-Chromatogram and MS-Spectrum for analogue 2 in accordance with an example.

FIG. 3f shows LC-Chromatogram and MS-Spectrum for analogue 3 in accordance with an example.

FIG. 3g shows LC-Chromatogram and MS-Spectrum for analogue 4 in accordance with an example.

FIG. 3h shows LC-Chromatogram and MS-Spectrum for analogue 5 in accordance with an example.

FIG. 3i shows LC-Chromatogram and MS-Spectrum for analogue 6 in accordance with an example.

FIG. 3j shows LC-Chromatogram and MS-Spectrum for analogue 6[1,4] in accordance with an example.

FIG. 3k shows LC-Chromatogram and MS-Spectrum for RgIA4[1,4] in accordance with an example.

FIG. 4a shows Stability of α-RgIA4 and analogue 6 in human serum. A) Human serum stability comparison. Values are the mean±SD of 3 separate replicates. B) Representative RP-HPLC trace of disulfide scrambling at different time points in human serum at 37° C. (Conc. 0.1 mg/mL). **p<0.01 in accordance with an example.

FIG. 4b shows determination of isomer by RP-HPLC co-injection. FIG. b-A shows synthesized RgIA4 and its [1,4] isomer; FIG. 4b-B shows synthesized analogue 6 and its [1,4]isomer; and FIG. 4b-C shows sequence representation of compounds tested in accordance with an example.

FIG. 5a shows NMR study of α-RgIA4, analogue 3 and 6. A) Secondary Hα chemical shift overlay. Residue No. 0 represents for Glu and No. 14 for Lys. B) Superposition of the representative NMR solution structure of B) α-RgIA4 (black) and 3 (red) C) α-RgIA4 (black) and 6 (blue) in accordance with an example.

FIGS. 5b to 5d show for α-RgIA4 an overlay of the amide regions of TOCSY (blue) and NOESY (red), HSQC aliphatic region, and aromatic region. Assignments were made using SPARKY. (CIR=citrulline, TIY=3-iodo-tyrosine) in accordance with an example.

FIG. 5e to 5g show for Analogue 3 an overlay of the amide regions of TOCSY (blue) and NOESY (red), HSQC aliphatic region and aromatic region. (CIR=citrulline) in accordance with an example.

FIG. 5h to 5j show for Analogue 6 an overlay of the amide regions of TOCSY (blue) and NOESY (red), HSQC aliphatic region and aromatic region. (CIR=citrulline, TIY=3-iodo-tyrosine) in accordance with an example.

FIG. 5k-A shows for RgIA4 backbone superimposition and FIG. 5k-B shows side chain superimposition with conformational restraints, atomic RMSD (2-12), and a Ramachandran plot in accordance with an example.

FIG. 5l-A shows, for Analogue 3, backbone superimposition; FIG. 5k-B shows side chain superimposition with conformational restraints, and FIG. 5k-C shows linker superimposition (green) with atomic RMSD (2-12), and a Ramachandran plot in accordance with an example.

FIG. 5m shows, for Analogue 6, backbone superimposition; FIG. 5m-B shows side chain superimposition with conformational restraints, and FIG. 5m-C shows linker superimposition (green) with atomic RMSD (2-12), and a Ramachandran plot in accordance with an example.

FIG. 6a shows selected binding model from docking of NMR structure ensemble of analogue 6 into a homology model of human (A, B) α9(+)/α9(−) and (C, D) α10(+)/α9(−) nAChR interface using RossetaDock. α9-ECD shown in green, α10-ECD in light blue and analogue 6 in orange. Binding residues are shown as stick representation with oxygen, nitrogen, and sulfur atoms in red, blue, and yellow, respectively. Dashed lines indicate hydrogen bonds formed between analogue 6 and receptors. The hα9-ECD structure was generated from RgIA bound X-ray crystal structure (PDB 6HY7) and hα10-ECD was generated from the previously reported homology model based on the same structure in accordance with an example.

FIG. 6b shows docking clustering files in accordance with an example.

FIG. 6c shows docking clustering files in accordance with an example.

FIG. 7a shows (A) Amino acid sequence alignments of α-Ctx RgIA, ImI, Vc1.1, PeIA, MII, and PnIA. Disulfide connectivity is Cys^{I}-Cys^{III} (loop I) and Cys^{II}-Cys^{IV} (loop II). #=C-terminal amide; \hat{ }=C-terminal carboxylate acid. (B) The binding surface of RgIA bound to α9 nAChR subunit crystal structure (PDB 6HY7). Binding residues (Ser4, Asp5, Arg7, and Arg9) are labeled in black font and shown as stick representation with oxygen, nitrogen, and sulfur atoms in red, blue, and yellow, respectively. (C) Chemical structures of native disulfide and developed disulfide mimetics. Pen=L-penicillamine. (D) Chemical syntheses of methylene thioacetal RgIA analogues in this study. All linear peptides were synthesized through Fmoc-SPPS on the automated synthesizer. Fully folded peptides were obtained via a two-operation procedure. Reaction conditions a) TCEP·HCl, K₂CO₃, H₂O; then Et3N, CH₂I₂, THF. b) I₂, AcOH, H₂O in accordance with an example.

FIG. 7b shows RP-HPLC analysis of the folding of RgIA-5524; FIG. 7b-A shows transformations of linear peptide to partial-folded and fully-folded analogue; and FIG. 7b-B shows HPLC traces for corresponding peptide in accordance with an example.

FIG. 7c shows LC-Chromatogram and MS-Spectrum for RgIA-5617 in accordance with an example.

FIG. 7d shows LC-Chromatogram and MS-Spectrum for RgIA-5533 in accordance with an example.

FIG. 7e shows LC-Chromatogram and MS-Spectrum for RgIA-5618 in accordance with an example.

FIG. 7f shows LC-Chromatogram and MS-Spectrum for RgIA-5524 in accordance with an example.

FIG. 7g shows LC-Chromatogram and MS-Spectrum for RgIA-5573 in accordance with an example.

FIG. 8a shows (A) Amino acid sequences and potencies of synthesized methylene thioacetal RgIA analogues on hα9α10 nAChRs. ^{a}All native disulfide or methylene thioacetal are connected in a Cys^{I}-Cys^{III}, Cys^{II}-Cys^{IV} configuration. Methylene thioacetal replacement is labeled in bold color with red for loop-I and green for loop-II. \hat{ } represents C-terminal carboxylic acid; Cit=L-citrulline; iY=L-3-iodo-tyrosine; bA=β-alanine; bhY=L-β-homotyrosine. ^{b}Calculated from concentration-response curves. Numbers in parenthesis are 95% confidence intervals. (B) Concentration-response analysis for inhibition of human α9α10 nAChR by synthesized peptides on blocking ACh-induced current on human nAChR currents expressed in Xenopus laevis oocytes. (C) IC₅₀ for inhibition of nAChR subtypes by RgIA-5524 and RgIA-5533 on blocking ACh-induced current on human nAChR currents expressed in Xenopus laevis oocytes. (D) Concentration-response analysis for inhibition of hα9α10 versus hα7 nAChR by RgIA-5524 and RgIA-5533. Data points represent the mean SEM from 3-4 independent experiments in accordance with an example.

FIG. 8b shows the effect (100 nM peptide) on blocking ACh induced current on human nAChR subtype currents expressed in X. laevis oocytes with Data points representing the mean±SEM from 3-4 independent experiments in accordance with an example.

FIG. 9 shows in vivo pain-relieving effects of RgIA-5524 in chronic chemotherapy-induced neuropathic pain. RgIA-5524 relieves pain induced by repeated oxaliplatin dosing. Mice were injected once per day, 5 days per week with the chemotherapeutic agent oxaliplatin (3.5 mg/kg, i.p.) over a three-week period. On the days of oxaliplatin injection, mice also received either saline or RgIA-5524 (40 μg/kg). Once per week, twenty-four hours after last RgIA-5524 injection, mice were assessed for cold allodynia using a cold plate as described in Experimental Sections. Allodynia reached statistical significance by day 21 and was effectively reversed by RgIA-5524. Statistical evaluations of the data were performed by one-way analysis of variance (ANOVA) followed by Dunnett's multiple comparison test. Data are expressed as means±SEM n=8 mice per group. ^{xxx}P<0.001 for significant difference from Ox/Sal vs Sal/Sal treated mice; ***P<0.001 for significant difference from Ox/Sal vs Ox/RgIA-5524 treated mice. Ox, oxaliplatin; Sal, 0.9% saline; s, seconds in accordance with an example.

FIG. 10 shows the α9 nAChR subunit are used for analgesic effects of RgIA-5524. (A, B) On day 1, a single dose of oxaliplatin 5 mg/kg i.p. was given along with either RgIA 5524 (40 ug/kg, s.c.) or 0.9% saline. Mice were assessed for cold allodynia on day 5. RgIA-5524 prevented the development of allodynia in (A) wildtype mice but not in (B) α9-subunit null mice (n=12 mice/group). (C, D) On day 1, mice were administered a higher dose of oxaliplatin (10 mg/kg i.p.) and either RgIA-5524 (40 ug/kg s.c.) or saline. Oxaliplatin-induced cold allodynia was prevented by the single dose of RgIA-5524 administered on day 1 in (C) wildtype mice but not in (D) α9-subunit null mice (n=8 mice/group). Statistical evaluations of the data were performed by one-way analysis of variance (ANOVA) followed by Dunnett's multiple comparison test. All results are expressed as means±SEM *P<0.05, **P<0.01, and ***P<0.001 for significant difference from Sal/Sal treated mice. ° P<0.05, ° ° P<0.01, and ° ° ° P<0.001 for significant difference from Ox/Sal treated mice. Ox, oxaliplatin; Sal, 0.9% saline; s, seconds, α9^{-/-}, α9 knockout mice in accordance with an example.

FIG. 11a shows (A) Binding activity of RgIA-5524 on other pain-related ion channels and receptors at 10 μM. ^{a}Each experiment was conducted with duplicate wells. Binding was calculated as % inhibition of the binding of a radioactively labeled ligand specific for each target and the enzyme inhibition effect was calculated as % inhibition of control enzyme activity. A secondary, concentration-response analysis was conducted when the screening assay indicated ≥50% inhibition. ^{b}nicotinic neuronal type. ^{c}strychnine sensitive. ^{d}strychnine insensitive; AR, adenosine receptor; AT, angiotensin; BK2, bradykinin receptor; CB, Cannabinoid receptor; CCK, cholecystokinin receptor; CRF, Corticotropin-releasing factor; D, dopamine; ET, endothelin receptor; GABA, γ-aminobutyric acid; GAL, galanin receptor; mGluR, metabotropic glutamate receptor; GlyR, Glycine receptor (strychnine-sensitive); H, histamine receptor; CysLT, cysteinyl leukotriene; M, muscarinic acetylcholine receptor; NK, neurokinin receptor; DOP, δ-opioid receptor; KOP, κ-opioid receptor; MOP, μ-opioid receptor; NOP, nociceptin/orphanin FQ receptor; GR, glucocorticoid receptor; ER, estrogen receptor; AR, androgen receptor; PAFR, platelet-activating factor receptor; TRH1, thyrotropin-releasing hormone; VPAC, vasoactive intestinal peptide receptor; V, vasopressin receptor; LTCC, L-type Ca²⁺ channel; NTCC, N-type Ca²⁺ channel; BZD, benzodiazepine; PCP, phencyclidine. (B) Functional activity of RgIA-5524 on GABAB receptors and hERG K⁺ channel. ^{e}Two separate experiments were conducted with duplicate wells for the IC₅₀ and EC₅₀ studies. Cellular agonist and antagonist effect were calculated as % of control response and % inhibition to a known reference agonist or antagonist, respectively; ^{f}Measured at 100 μM of RgIA-5524. (C) Enzyme and uptake assays of RgIA-5524. ^{g}Each experiment was conducted with duplicate wells. The antagonist effect was calculated as % inhibition to the measured component. TXA₂ synthetase, Thromboxane A2 synthetase; constitutive NOS, constitutive NO synthase; MAO, monoamine oxidase. Concentration-response analysis of (D) agonist and (E) antagonist effect of RgIA-5524 on GABAB receptors. (F) Concentration-response analysis of RgIA-5524 on hERG K⁺ channel measured by tail current inhibition. (G) Inhibition of CYP enzyme isoforms by RgIA-5524 at 100 nM and 10 μM. Duplicated experiments were conducted for each concentration and data are expressed as means±SEM in accordance with an example.

FIG. 11b to 11d show for RgIA-5533 an overlay of the amide regions of TOCSY (blue) and NOESY (red), HSQC aliphatic region and aromatic region. Assignments were made using SPARKY. (SCS=L-S-methylene-Cys, CIR=citrulline, TIY=3-iodo-tyrosine) in accordance with an example.

FIG. 11e to 11g show for RgIA-5617 an overlay of the amide regions of TOCSY (blue) and NOESY (red), HSQC aliphatic region and aromatic region. Assignments were made using SPARKY. (SCS=L-S-methylene-Cys, CIR=citrulline, TIY=3-iodo-tyrosine) in accordance with an example.

FIG. 11h to 11j show for RgIA-5524 an overlay of the amide regions of TOCSY (blue) and NOESY (red), HSQC aliphatic region and aromatic region. (SCS=L-S-methylene-Cys, CIR=citrulline, TIY=3-iodo-tyrosine, BHY=L-beta-homotyrosine) in accordance with an example.

FIG. 11k shows for RgIA-5533 A) Backbone superimposition and B) Side chain superimposition with conformational restraints, atomic RMSD (2-12), and a Ramachandran plot in accordance with an example.

FIG. 11l shows for RgIA-5617 A) Backbone superimposition and B) Side chain superimposition with conformational restraints, atomic RMSD (2-12), and a Ramachandran plot in accordance with an example.

FIG. 11m shows for RgIA-5524 A) Backbone superimposition and B) Side chain superimposition with conformational restraints, atomic RMSD (2-12), and a Ramachandran plot in accordance with an example.

FIG. 12 shows (A) Overlay of the Secondary-chemical-shifts of RgIA (black), RgIA4 (grey), RgIA-5617 (pink), RgIA-5533 (green) and RgIA-5524 (blue). The x-axis shows the peptide sequences with substituted residue for the mutant at residue 4, 9, 10, 13 and 14 calculated based on their corresponding standard chemical shifts. Representative NMR structures and distance measurements (Å) between Cα in two intramolecular bridges. (B) RgIA bound to hα9 nAChR subunit crystal structure (PDB 6HY7); Representative NMR solution structures of (C) RgIA (PDB 2JUQ); (D) RgIA4; (E) RgIA-5533; (F) RgIA-5617 and (G) RgIA-5524. Structures are shown as stick representation with atom oxygen, nitrogen, sulfur and iodine colored in red, blue, yellow and purple, respectively. Cα distances were measured by PyMOL program in accordance with an example.

FIG. 13 shows RgIA-5524 shows greatly enhanced stability compared with RgIA4. (A) Complete disulfide scrambling prevention was observed indicated by HPLC traces at certain time points post peptide incubation in 90% human serum at 37° C. The front peak in left panel is the scrambled isomer RgIA4[1,4]. (B) Stability assay of RgIA-5524 and RgIA-5533 vs. RgIA4 in human serum. Peptides were incubated in 90% human serum AB type (0.1 mg/mL) at 37° C. (C) Reductive stability assay of RgIA-5524 vs. RgIA4. Peptide samples were dissolved at 0.1 mg/mL in the presence of reduced GSH (10 equiv.) in pH 7.4 PBS and incubated at 37° C. Statistical evaluations of the data were performed by student t (unpaired) test. All results are expressed as means±SD (n=3), **P<0.01, ***P<0.001 in accordance with an example.

Reference will now be made to the exemplary embodiments illustrated, and specific language will be used herein to describe the same. It will nevertheless be understood that no limitation of the scope of the disclosure is thereby intended.

### DETAILED DESCRIPTION

While these exemplary embodiments are described in sufficient detail to enable those skilled in the art to practice the disclosure, it should be understood that other embodiments may be realized and that various changes to the disclosure may be made without departing from the spirit and scope of the present disclosure. Thus, the following more detailed description of the embodiments of the present disclosure is not intended to limit the scope of the disclosure, as claimed, but is presented for purposes of illustration only and not limitation to describe the features and characteristics of the present disclosure, to set forth the best mode of operation of the disclosure, and to sufficiently enable one skilled in the art to practice the disclosure. Accordingly, the scope of the present disclosure is to be defined solely by the appended claims.

### Definitions

In describing and claiming the present disclosure, the following terminology will be used.

The singular forms “a,” “an,” and “the” include plural referents unless the context clearly dictates otherwise. Thus, for example, reference to “a peptide” includes reference to one or more of such structures and reference to “the analog” refers to one or more of such analogs.

As used herein, the term “substantially” refers to the complete or nearly complete extent or degree of an action, characteristic, property, state, structure, item, or result. For example, an object that is “substantially” enclosed would mean that the object is either completely enclosed or nearly completely enclosed. The exact allowable degree of deviation from absolute completeness may in some cases depend on the specific context. However, generally speaking the nearness of completion will be so as to have the same overall result as if absolute and total completion were obtained. The use of “substantially” is equally applicable when used in a negative connotation to refer to the complete or near complete lack of an action, characteristic, property, state, structure, item, or result. For example, a composition that is “substantially free of” particles would either completely lack particles, or so nearly completely lack particles that the effect would be the same as if it completely lacked particles. In other words, a composition that is “substantially free of” an ingredient or element may still actually contain such item as long as there is no measurable effect thereof.

As used herein, the term “about” is used to provide flexibility and imprecision associated with a given term, metric or value. Unless otherwise stated, use of the term “about” in accordance with a specific number or numerical range should also be understood to provide support for such numerical terms or range without the term “about”. For example, for the sake of convenience and brevity, a numerical range of “about 50 angstroms to about 80 angstroms” should also be understood to provide support for the range of “50 angstroms to 80 angstroms.” Furthermore, it is to be understood that in this written description support for actual numerical values is provided even when the term “about” is used therewith. For example, the recitation of “about” 30 should be construed as not only providing support for values a little above and a little below 30, but also for the actual numerical value of 30 as well. The degree of flexibility for a particular variable can be readily determined by one skilled in the art. However, unless otherwise enunciated, the term “about” generally connotes flexibility of less than 2%, and most often less than 1%, and in some cases less than 0.01%.

As used herein, the terms “treat,” “treatment,” or “treating” and the like refers to administration of a therapeutic agent or therapeutic action to a subject who is either asymptomatic or symptomatic. In other words, “treat,” “treatment,” or “treating” can refer to the act of reducing or eliminating a condition (i.e., symptoms manifested), or it can refer to prophylactic treatment (i.e., administering to a subject not manifesting symptoms in order to prevent their occurrence). Such prophylactic treatment can also be referred to as prevention of the condition, preventative action, preventative measures, and the like.

As used herein, the terms “therapeutic agent,” “active agent,” and the like can be used interchangeably and refer to agent that can have a beneficial or positive effect on a subject when administered to the subject in an appropriate or effective amount. In one aspect, the therapeutic or active agent can be an α-RgIA4 peptide analog. The terms “additional active agent,” “supplemental active agent,” “secondary active agent,” and the like can be used interchangeably and refer to a compound, molecule, or material other than be an α-RgIA4 peptide analog.

As used herein, the terms “formulation” and “composition” are used interchangeably and refer to a mixture of two or more compounds, elements, or molecules. In some aspects, the terms “formulation” and “composition” may be used to refer to a mixture of one or more active agents with a carrier or other excipients.

Furthermore, the term “dosage form” can include one or more formulation(s) or composition(s) provided in a format (e.g. a specific form, shape, vehicle, etc.) for administration to a subject. For example, an “oral dosage form” can be suitable for administration to a subject's mouth. A “topical dosage form” can be suitable for administration to a subject's skin by rubbing, etc.

As used herein, a “treatment situs” refers to a location on or within a subject where treatment is desired. For example, when treating pain, the treatment situs can be the area of the pain. Further, as used herein, an “application situs” refers to a location on or in a subject where treatment is administered. Further, the application situs for an infusion dosage formulation may be an area where the infusion equipment enters the subject's circulatory system. Yet further, the application situs for a topical dosage formulation may be the area of skin or mucosa to which the topical dosage formulation is applied. In some embodiments, the application situs may be substantially the same as the treatment situs (e.g., the composition or formulation is administered directly to the treatment site). In other embodiments, the application situs may be different from (e.g., distal from) the treatment situs. In such cases even though administration may be distal from the treatment situs, the composition or formulation still exerts a therapeutic effect at the treatment situs.

As used herein, “topical composition” or “topical administration” and the like refer to a composition suitable for administration directly to a skin or mucosa surface and from which an effective amount of a drug is released. In some embodiments, topical compositions can provide a local or localized therapeutic effect (e.g. at or near an application situs). For example, a topical composition when applied to a wound, a lesion, a burn, a canker sore, etc. (e.g. a treatment situs), may primarily exert a therapeutic effect at or around the application situs, but not substantially beyond it. In other embodiments, a topical composition can provide a regional effect. For example, a topical composition administered to a skin surface on a region of the body, such as a finger, arm, ankle, joint, etc. can exert a therapeutic effect within the region, but not substantially beyond. For example, a topical composition administered to the region of an ankle can have a therapeutic effect in and around the ankle, by for example, reducing edema, joint inflammation, pain, etc. In other embodiments, topical compositions can provide a systemic effect. In some aspects, a topical composition can provide the therapeutic effect though a mechanism of action where the drug or active agent itself arrives at the treatment situs. In other aspects, the topical composition can provide the therapeutic effect through an intermediate mechanism of action, such as biochemical cascade event, such as an enzymatic cascade or other signaling (e.g. cellular signaling, or inter/intra cellular signaling) event which ultimately exerts the desired therapeutic effect at the treatment situs. In some examples, such intermediate mechanism can allow treatment of a treatment situs that is distal from an application situs. In yet other examples, when treatment of a distal treatment situs occurs, the active agent may travel through dermal and other tissues from the application situs to the treatment situs and exert a direct effect.

As used herein, “transdermal” refers to the route of administration of a therapeutic agent through an unbroken skin surface when administered to the skin surface. When transdermally administered, the drug or active agent migrates from the application situs to a treatment situs and exerts a therapeutic effect. Transdermal compositions and dosage forms can include structures and/or devices which assist in holding the composition on a skin surface, such as, for example, backing films, adhesives, reservoirs, etc. Furthermore, transdermal compositions can include agents which aid or otherwise facilitate movement of the active agent from an application situs to a treatment situs (e.g., through the skin and into the subject's circulatory system) such as penetration or permeation enhancers. Such penetration or permeation enhancers can also be used with topical formulations in some embodiments.

The term “skin” or “skin surface” includes not only the outer skin of a subject comprising one or more epidermal layers, but also mucosal surfaces such as the mucosa of the respiratory (including nasal and pulmonary), oral (mouth and buccal), vaginal, and rectal cavities. Hence, the term “transdermal” may encompass “transmucosal” as well.

As used herein, “co-administering” a first therapeutic agent with a second therapeutic agent can include concomitant administration within a suitable time window. In one example, the suitable time window can be less than one or more of: 1 hour, 45 minutes, 30 minutes, 15 minutes, 5 minutes, 2 minutes, 1 minute, or combinations thereof. Concomitant administration can be from the same composition or from different compositions.

As used herein, a “subject” refers to a mammal that may benefit from the method or device disclosed herein. Examples of subjects include humans, and may also include other animals such as horses, pigs, cattle, dogs, cats, rabbits, and aquatic mammals. In one specific aspect, the subject is a human.

As used herein, a “dosing regimen” or “regimen” such as an “initial dosing regimen” or “starting dose” or a “maintenance dosing regimen” refers to how, when, how much, and for how long a dose of the compositions of the present disclosure can be administered to a subject. For example, an initial or starting dose regimen for a subject may provide for a total daily dose of from about 15 mcg/1 mL to about 1500 mcg/1 mL administered in two divided doses at least 12 hours apart (e.g., once with breakfast and once with dinner) with meals repeated daily for 30 days.

As used herein, “daily dose” refers to the amount of active agent (e.g., an α-RgIA4 peptide analog) administered to a subject over a 24-hour period of time. The daily dose can be administered two or more administrations during the 24-hour period. In one embodiment, the daily dose provides for two administrations in a 24-hour period. With this in mind, an “initial dose” or initial daily dose” refers to a dose administered during the initial regimen or period of a dosing regimen.

As used herein, an “effective amount” or a “therapeutically effective amount” of a drug refers to a non-toxic, but sufficient amount of the drug, to achieve therapeutic results in treating a condition for which the drug is known to be effective. It is understood that various biological factors may affect the ability of a substance to perform its intended task. Therefore, an “effective amount” or a “therapeutically effective amount” may be dependent in some instances on such biological factors. Further, while the achievement of therapeutic effects may be measured by a physician or other qualified medical personnel using evaluations known in the art, it is recognized that individual variation and response to treatments may make the achievement of therapeutic effects a somewhat subjective decision. The determination of an effective amount is well within the ordinary skill in the art of pharmaceutical sciences and medicine. See, for example, Meiner and Tonascia, “Clinical Trials: Design, Conduct, and Analysis,” Monographs in Epidemiology and Biostatistics, Vol. 8 (1986), incorporated herein by reference.

As used herein, an “acute” condition refers to a condition that can develop rapidly and have distinct symptoms needing urgent or semi-urgent care. By contrast, a “chronic” condition refers to a condition that is typically slower to develop and lingers or otherwise progresses over time. Some examples of acute conditions can include without limitation, an asthma attack, bronchitis, a heart attack, pneumonia, and the like. Some examples of chronic conditions can include without limitation, arthritis, diabetes, hypertension, high cholesterol, and the like.

As used herein, “selectivity” refers to modifying an action that provides a difference within a group (e.g., a group of cells) or between groups (e.g., a group of non-viable cells and a group of viable cells). For example, the action can be receptor binding and the groups can be a first receptor and a second receptor. For example, “selective receptor binding” of a first receptor compared to a second receptor can provide a difference between the first receptor and the second receptor at a selectivity ratio. In one example, the selectivity ratio differs from a 1:1 ratio. In one example, the selectivity ratio can be a ratio that is greater than at least one of: 1:1, 2:1, 3:1: 4:1, 5:1, 6:1, 7:1, 8:1, 9:1, 10:1, 15:1, 20:1, 25:1, 30:1, 35:1, 40:1, 45:1, 50:1, 100:1, the like, and a combination thereof.

As used herein “D-substituted analogs” include RgIA and RgIA4 analogs disclosed herein having one or more L-amino acids substituted with D-amino acids. The D-amino acid can be the same amino acid type as that found in the analog sequence or can be a different amino acid. Accordingly, D-analogs are also Variants.

As used herein “Variants” include RgIA analogs disclosed herein wherein one or more amino acids have been replaced with a non-amino acid component, or where the amino acid has been conjugated to a functional group or a functional group has been otherwise associated with an amino acid. The modified amino acid may be, e.g., a glycosylated amino acid, a PEGylated amino acid (covalent and non-covalent attachment or amalgamation of polyethylene glycol (PEG) polymers), a farnesylated amino acid, an acetylated amino acid, an acylated amino acid, a biotinylated amino acid, a phosphorylated amino acid, an amino acid conjugated to a lipid moiety such as a fatty acid, or an amino acid conjugated to an organic derivatizing agent. The presence of modified amino acids may be advantageous in, for example, (a) increasing polypeptide serum half-life and/or functional in vivo half-life, (b) reducing polypeptide antigenicity, (c) increasing polypeptide storage stability, (d) increasing peptide solubility, (e) prolonging circulating time, and/or (f) increasing bioavailability, e.g. increasing the area under the curve (AUCsc). Amino acid(s) can be modified, for example, co-translationally or post-translationally during recombinant production (e.g., N-linked glycosylation at N-X-S/T motifs during expression in mammalian cells) or modified by synthetic means. The modified amino acid can be within the sequence or at the terminal end of a sequence. Variants can include derivatives as described elsewhere herein.

As used herein, “I-3-Y” is 3-iodo-tyrosine, and “3-R-tyrosine” and “R-3-Y” is a peptide residue selected from the group consisting of 3-chloro-tyrosine, 3-fluoro-tyrosine, 3-iodo-tyrosine, and tyrosine.

As used herein, “Cit” is citrulline.

As used herein, “iY” is L-3-iodo-tyrosine.

As used herein, “Dap” is L-2,3-diaminopropionic acid.

As used herein, “^{b}A” and “bA” is β-alanine.

As used herein, “bhY” is beta-homotyrosine.

As used herein, “Xaa” is any amino acid. Moreover, as used in this written description, Xaa provides express support for any amino acid. For example, Xaa provides express support for any amino acid or derivative thereof. For example, Xaa provides support for: alanine (Ala or A), arginine (Arg or R), asparagine (Asn or N), aspartic acid (Asp or D), cysteine (Cys or C), glutamic acid (Gly or E), glutamine (Gln or Q), glycine (Gly or G), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Tyr or W), Tyrosine (Tyr or Y), Valine (Val or V), selenosysteine (Sec or U), pyrrolysine (Pyl or O), the like, or a combination thereof.

As used herein, “Variants of RgIA analogs” or “Variants of RgIA-4 analogs” disclosed herein include peptides having one or more amino acid additions, deletions, or substitutions, as compared to an RgIA peptide disclosed herein or an RgIA-4 peptide disclosed herein.

Embodiments disclosed herein include the RgIA analogs described herein as well as variants, D-substituted analogs, modifications, and derivatives of the RgIA analogs described herein. In some embodiments, variants, D-substituted analogs, modifications, and derivatives have 1, 2, 3, 4, 5, 6, 7, 8, 9, 10, 11, 12, 13, 14, or 15 sequence additions, deletions, substitutions, replacements, conjugations, associations, or permutations. Each analog peptide disclosed herein may also include additions, deletions, substitutions, replacements, conjugations, associations, or permutations at any position including positions 1, 2, 3, 4, 5, 6, 7, 8, 9, 10, 11, 12, 13, 14, or 15 of an analog peptide sequence disclosed herein.

In some embodiments an Xaa position can be included in any position of an analog peptide, wherein Xaa represents an addition, deletion, substitution, replacement, conjugation, association or permutation. In particular embodiments, each analog peptide has 1, 2, 3, 4, 5, 6, 7, 8, 9, 10, 11, 12, 13, 14, or 15 Xaa positions at one or more of positions 1, 2, 3, 4, 5, 6, 7, 8, 9, 10, 11, 12, 13, 14, or 15.

An analog can have more than one change (addition, deletion, substitution, replacement, conjugation, association, or permutation) and qualify as one or more of a variant, D-substituted analog, modification, and/or derivative. That is, inclusion of one classification of analog, variant, D-substituted analog, modification and/or derivative is not exclusive to inclusion in other classifications and all are collectively referred to as “analog peptides” herein.

An amino acid substitution can be a conservative or a non-conservative substitution. Variants of RgIA analogs disclosed herein can include those having one or more conservative amino acid substitutions. As used herein, a “conservative substitution” involves a substitution found in one of the following conservative substitutions groups: Group 1: alanine (Ala or A), glycine (Gly or G), serine (Ser or S), threonine (Thr or T); Group 2: aspartic acid (Asp or D), glutamic acid (Glu or E); Group 3: asparagine (Asn or N), glutamine (Gln or Q); Group 4: arginine (Arg or R), lysine (Lys or K), histidine (His or H); Group 5: isoleucine (Ile or I), leucine (Leu or L), methionine (Met or M), valine (Val or V); and Group 6: phenylalanine (Phe or F), tyrosine (Tyr or Y), tryptophan (Trp or W).

Additionally, amino acids can be grouped into conservative substitution groups by similar function, chemical structure, or composition (e.g., acidic, basic, aliphatic, aromatic, sulfur-containing). For example, an aliphatic grouping may include, for purposes of substitution, Gly, Ala, Val, Leu, and Ile. Other groups containing amino acids that are considered conservative substitutions for one another include: sulfur-containing: Met and Cys; acidic: Asp, Glu, Asn, and Gln; small aliphatic, nonpolar or slightly polar residues: Ala, Ser, Thr, Pro, and Gly; polar, negatively charged residues and their amides: Asp, Asn, Glu, and Gln; polar, positively charged residues: His, Arg, and Lys; large aliphatic, nonpolar residues: Met, Leu, Ile, Val, and Cys; and large aromatic residues: Phe, Tyr, and Trp. Additional information is found in Creighton (1984) Proteins, W.H. Freeman and Company.

As used herein, a “positive amino acid” includes the proteinogenic positive amino acids His, Arg, and Lys and the non-proteinogenic positive amino acids.

As used herein, an “aromatic amino acid” includes the proteinogenic aromatic amino acids Phe, Tyr, and Trp and the non-proteinogenic aromatic amino acids.

Variants of RgIA analogs or RgIA-4 analogs disclosed or referenced herein also include sequences with at least 70% sequence identity, at least 80% sequence identity, at least 85% sequence identity, at least 90% sequence identity, at least 95% sequence identity, at least 96% sequence identity, at least 97% sequence identity, at least 98% sequence identity, or at least 99% sequence identity to a peptide sequence disclosed or referenced herein. More particularly, variants of the RgIA analogs or RgIA-4 analogs disclosed herein include peptides that share: 70% sequence identity with any of SEQ ID NOs:1-13; 80% sequence identity with any of SEQ ID NOS:1-13; 81% sequence identity with any of SEQ ID NOs: 1-13; 82% sequence identity with any of SEQ ID NO: 1-13; 83% sequence identity with any of SEQ ID NOs: 1-13; 84% sequence identity with any of SEQ ID NO: 1-13; 85% sequence identity with any of SEQ ID NO: 1-13; 86% sequence identity with any of SEQ ID NO: 1-13; 87% sequence identity with any of SEQ ID NO: 1-13; 88% sequence identity with any of SEQ ID NO: 1-13; 89% sequence identity with any of SEQ ID NO: 1-13; 90% sequence identity with any of SEQ ID NO: 1-13; 91% sequence identity with any of SEQ ID NO: 1-13; 92% sequence identity with any of SEQ ID NO: 1-13; 93% sequence identity with any of SEQ ID NO: 1-13; 94% sequence identity with any of SEQ ID NO:1-13; 95% sequence identity with any of SEQ ID NO: 1-13; 96% sequence identity with any of SEQ ID NO: 1-13; 97% sequence identity with any of SEQ ID NO: 1-13; 98% sequence identity with any of SEQ ID NO: 1-13; or 99% sequence identity with any of SEQ ID NO: 1-13.

The C-terminus of a synthetic analgesic peptide may be a carboxylic acid or an amide group. The present disclosure also relates to RgIA analogs further modified by (i) additions made to the C-terminus, such as tyrosine, 3-iodo-tyrosine, a fluorescent tag, lipids, carbohydrates, or beta-homo amino acids, D/L-sulfono-γ-AApeptides, L-γ-AApeptides, and/or (ii) additions made to the N-terminus, such as tyrosine, 3-iodo-tyrosine, pyroglutamate, a fluorescent tag, lipids, carbohydrates, or beta-homo amino acids.

As used herein, the term “gene” refers to a nucleic acid sequence that encodes a peptide. This definition includes various sequence polymorphisms, mutations, and/or sequence variants wherein such alterations do not affect the function of the encoded peptide. The term “gene” may include not only coding sequences but also regulatory regions such as promoters, enhancers, and termination regions. “Gene” further can include all introns and other DNA sequences spliced from the mRNA transcript, along with variants resulting from alternative splice sites. Nucleic acid sequences encoding the peptide can be DNA or RNA that directs the expression of the peptide. These nucleic acid sequences may be a DNA strand sequence that is transcribed into RNA or an RNA sequence that is translated into protein. The nucleic acid sequences include both the full-length nucleic acid sequences as well as non-full-length sequences derived from the full-length protein. The sequences can also include degenerate codons of the native sequence or sequences that may be introduced to provide codon preference in a specific cell type. Gene sequences to encode peptides disclosed herein are available in publicly available databases and publications.

As used herein, recitation of a specific amino acid also includes support for the specific amino acid and any analog, variant, D-substituted analog, modification and/or derivatives thereof. In one example, recitation of tyrosine also explicitly includes support for 3-chloro-tyrosine, 3-fluoro-tyrosine, 3-iodo-tyrosine, tyrosine, ortho-tyrosine, 3-nitro-tyrosine, 3-amino-tyrosine, O-methyl-tyrosine, 2,6-dimethyl-tyrosine, beta-homo-tyrosine, Boc-Tyr(3,5-I₂)-OSu, [CpRu(Fmoc-tyrosin)]CF₃CO₂, O-(2-Nitrobenzyl)-L-tyrosine hydrochloride, 3-Nitro-L-tyrosine ethyl ester hydrochloride, N-(2,2,2-trifluoromethyl)-L-Tyrosine Ethyl Ester, DL-o-Tyrosine, the like, or a combination thereof. In one example, recitation of cysteine also explicitly includes support for cysteine, L-cysteic acid monohydrate, L-cysteinesulfinic acid monohydrate, seleno-L-cystine, the like, or a combination thereof. In one example, recitation of lysine also explicitly includes support for Fmoc-Lys(Me,Boc)-OH, Fmoc-Lys(Me)₃-OH Chloride, Fmoc-L-Lys(Nvoc)-OH, Fmoc-Lys(palmitoyl)-OH, Fmoc-L-Photo-Lysine, DL-5-Hydroxylysine hydrochloride, H-L-Photo-lysine HCl, the like, or a combination thereof.

In this disclosure, “comprises,” “comprising,” “containing” and “having” and the like can have the meaning ascribed to them in U.S. patent law and can mean “includes,” “including,” and the like, and are generally interpreted to be open ended terms. The terms “consisting of” or “consists of” are closed terms, and include only the components, structures, steps, or the like specifically listed in conjunction with such terms, as well as that which is in accordance with U.S. patent law. “Consisting essentially of” or “consists essentially of” have the meaning generally ascribed to them by U.S. patent law. In particular, such terms are generally closed terms, with the exception of allowing inclusion of additional items, materials, components, steps, or elements, that do not materially affect the basic and novel characteristics or function of the item(s) used in connection therewith. For example, trace elements present in a composition, but not affecting the compositions nature or characteristics would be permissible if present under the “consisting essentially of” language, even though not expressly recited in a list of items following such terminology. When using an open-ended term, like “comprising” or “including,” in the written description it is understood that direct support should be afforded also to “consisting essentially of” language as well as “consisting of” language as if stated explicitly and vice versa.

The terms “first,” “second,” “third,” “fourth,” and the like in the description and in the claims, if any, are used for distinguishing between similar elements and not necessarily for describing a particular sequential or chronological order. It is to be understood that any terms so used are interchangeable under appropriate circumstances such that the embodiments described herein are, for example, capable of operation in sequences other than those illustrated or otherwise described herein. Similarly, if a method is described herein as comprising a series of steps, the order of such steps as presented herein is not necessarily the only order in which such steps may be performed, and certain of the stated steps may possibly be omitted and/or certain other steps not described herein may possibly be added to the method.

As used herein, comparative terms such as “increased,” “decreased,” “better,” “worse,” “higher,” “lower,” “enhanced,” “improved,” “maximized,” “minimized,” and the like refer to a property of a device, component, composition, biologic response, biologic status, or activity that is measurably different from other devices, components, compositions, biologic responses, biologic status, or activities that are in a surrounding or adjacent area, that are similarly situated, that are in a single device or composition or in multiple comparable devices or compositions, that are in a group or class, that are in multiple groups or classes, or as compared to an original (e.g. untreated) or baseline state, or the known state of the art. For example, an α-RgIA4 analog with “improved” performance in reducing neurologic pain would present an improvement with respect to at least one aspect of stability, binding efficacy, potency, or other performance related property as compared to other α-RgIA4 analogs.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary.

As used herein, the term “at least one of” is intended to be synonymous with “one or more of” For example, “at least one of A, B and C” explicitly includes only A, only B, only C, or combinations of each.

Concentrations, amounts, levels and other numerical data may be expressed or presented herein in a range format. It is to be understood that such a range format is used merely for convenience and brevity and thus should be interpreted flexibly to include not only the numerical values explicitly recited as the limits of the range, but also to include all the individual numerical values or sub-ranges or decimal units encompassed within that range as if each numerical value and sub-range is explicitly recited. As an illustration, a numerical range of “about 1 to about 5” should be interpreted to include not only the explicitly recited values of about 1 to about 5, but also include individual values and sub-ranges within the indicated range. Thus, included in this numerical range are individual values such as 2, 3, and 4 and sub-ranges such as from 1-3, from 2-4, and from 3-5, etc., as well as 1, 2, 3, 4, and 5, individually. This same principle applies to ranges reciting only one numerical value as a minimum or a maximum. Furthermore, such an interpretation should apply regardless of the breadth of the range or the characteristics being described.

Any steps recited in any method or process claims may be executed in any order and are not limited to the order presented in the claims. Means-plus-function or step-plus-function limitations will only be employed where for a specific claim limitation all of the following conditions are present in that limitation: a) “means for” or “step for” is expressly recited; and b) a corresponding function is expressly recited. The structure, material or acts that support the means-plus function are expressly recited in the description herein. Accordingly, the scope of the invention should be determined solely by the appended claims and their legal equivalents, rather than by the descriptions and examples given herein.

The term “coupled,” as used herein, is defined as directly or indirectly connected in a chemical, mechanical, electrical or nonelectrical manner. Objects described herein as being “adjacent to” each other may be in physical contact with each other, in close proximity to each other, or in the same general region or area as each other, as appropriate for the context in which the phrase is used.

Occurrences of the phrase “in one embodiment,” or “in one aspect,” herein do not necessarily all refer to the same embodiment or aspect. Reference throughout this specification to “an example” means that a particular feature, structure, or characteristic described in connection with the example is included in at least one embodiment. Thus, appearances of the phrases “in an example” in various places throughout this specification are not necessarily all referring to the same embodiment.

### EXAMPLE EMBODIMENTS

An initial overview of disclosure embodiments is provided below and specific embodiments are then described in further detail. This initial summary is intended to aid readers in understanding the technological concepts more quickly, but is not intended to identify key or essential features thereof, nor is it intended to limit the scope of the claimed subject matter.

Conotoxins (CTxs), derived from the venom of marine predatory cone snails, are promising candidates for non-opioid analgesics due to their high potency and selectivity for ion channels implicated in neuropathic pain. ω-conotoxin MVIIA, also known as Ziconotide (Prialt®), selectively targets voltage-gated calcium channel subtype (Cav2.2), was approved by the U.S. FDA in 2004, and has been used clinically for the treatment of intractable chronic pain.

Nicotinic acetylcholine receptors (nAChRs), distributed throughout both peripheral and central nervous systems, are a group of transmembrane ligand-gated cationic channels that mediate fast synaptic transmission and are involved in a wide range of nervous system disorders including neuropathic pain, Parkinson's disease, schizophrenia, alcohol, and drug addiction. Different nAChR subunits including α, β, γ, δ, and ε associate in various combinations within these homo- or hetero-pentameric receptors, leading to a complex variety of nAChR subtypes with distinct pharmacological and biophysical functions. The nAChRs have previously been targeted for analgesic drug discovery albeit with progress being hindered by a narrow therapeutic window and side effects caused by indiscriminate subtype targeting.

Recent studies have identified inhibition of the α9α10 nicotinic acetylcholine receptor (nAChR) subtype as a potential non-opioid based mechanism for chemotherapy-induced neuropathic pain. Among the α9α10 nAChR antagonists, the second generation analogue α-RgIA4, modified from the parent sequence α-RgIA, crosses over the “species-related affinity gap” and exhibits high potency for both rodent (IC₅₀ 0.9 nM) and human α9α10 nAChR (IC₅₀ 1.5 nM) without inhibiting other subtypes and other pain-related receptors (E.g., selectivity >1000 fold). Therefore, α-RgIA4 has potential as a lead compound for non-opioid analgesic development.

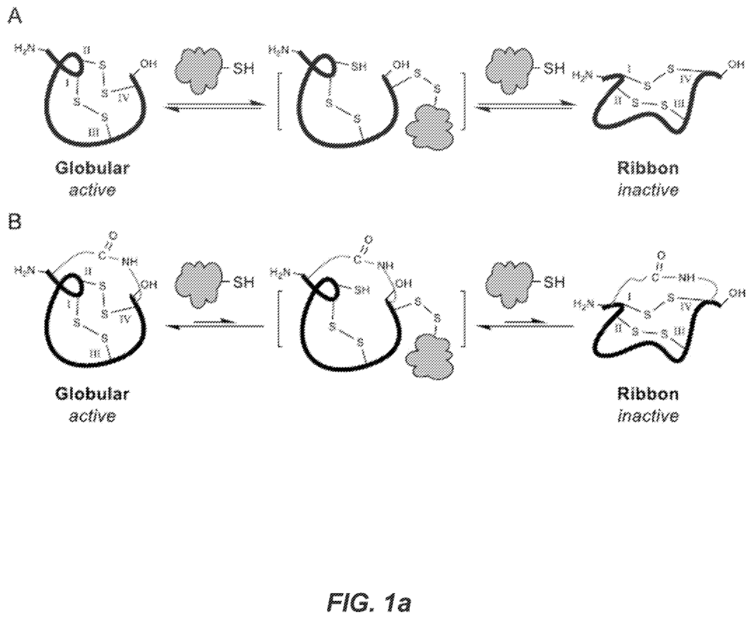

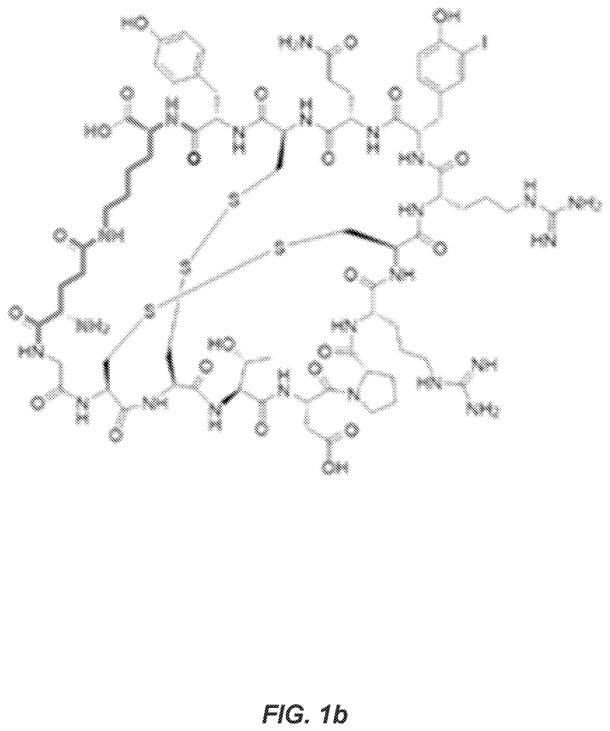

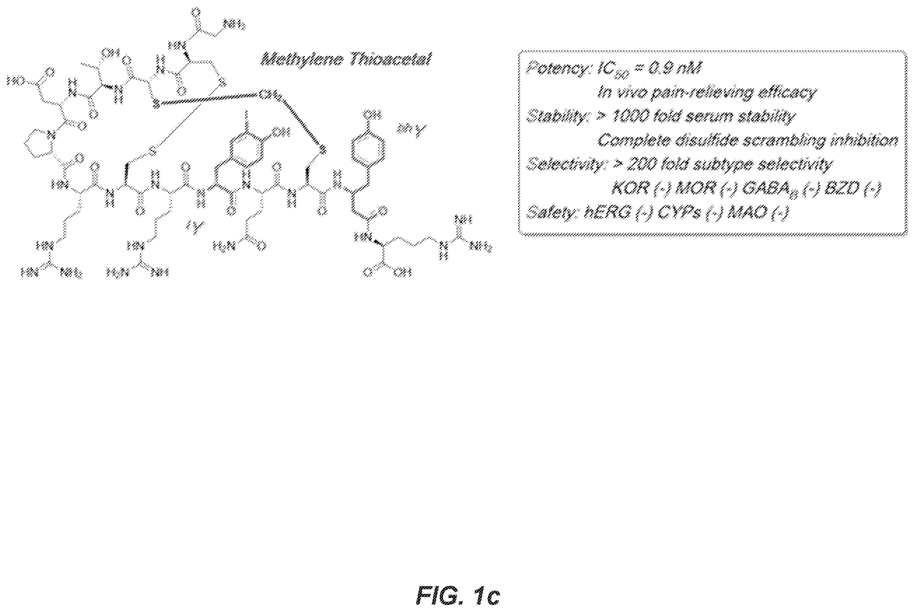

However, as with other disulfide-rich peptide drug molecules, α-RgIA4 is a poor candidate because of its low protease resistance and short plasma half-life. This is caused by disulfide scrambling induced by thiol/disulfide exchange reactions, which result in conformational changes and substantial loss of potency due to marginal differences in the thermodynamic stabilities between the active and inactive conformations, as shown in FIG. 1b-A. Structurally, CTxs rely on highly conserved cysteine frameworks to maintain rigid structures, which are crucial for receptor recognition, potency, and selectivity. Unfortunately, like other disulfide-rich peptides, RgIA4 is susceptible to disulfide-scrambling in reducing physiological environments that can lead to concomitant alternation of three-dimensional structures, aggregation, decreased therapeutic efficacy, and increased immunogenic side effects.

Disulfide mimetics have attempted to address this issue and produce bioavailable compounds for further clinical developments. However, disulfide mimetics may cause structural perturbation and therefore result in potency loss. For example, α-RgIA analogues having non-reducible dicarba bridges in place of native disulfides are not subject to disulfide scrambling but have significantly (i.e., two orders of magnitude) reduced potency compared with the native peptide. “Head-to-tail” backbone cyclization has been attempted as another method for CTxs stabilization by hiding the flexible terminal protease recognition regions. For example, cRgIA-6, together with other backbone cyclized analogues have exhibited increases in serum stability; however, this increased stability has resulted in reduced potency for human α9α10 nAChR.

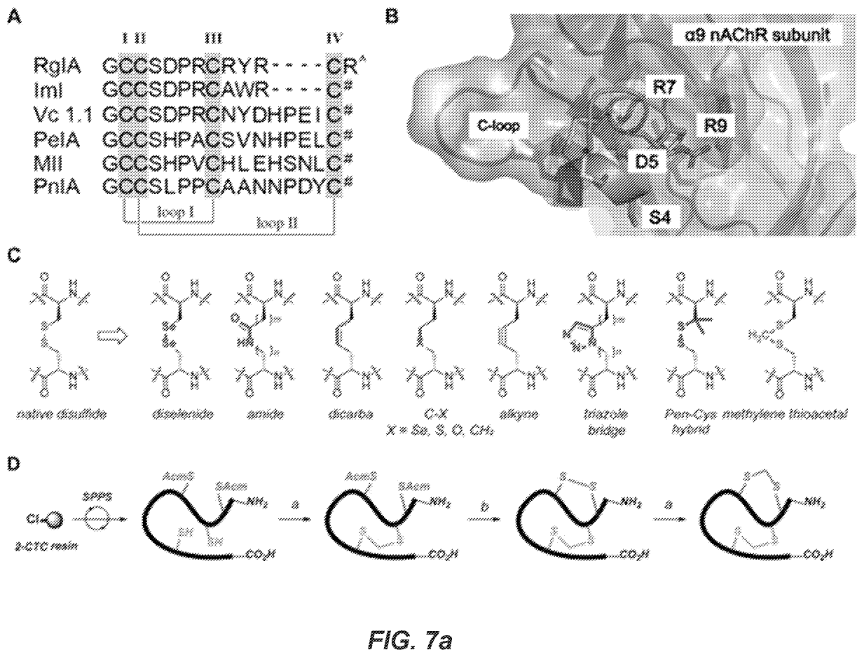

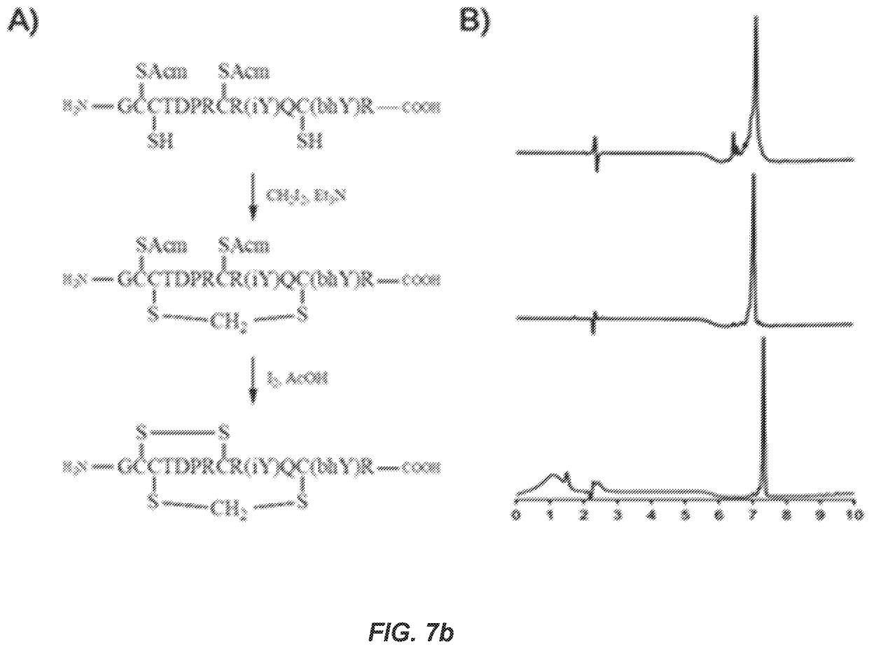

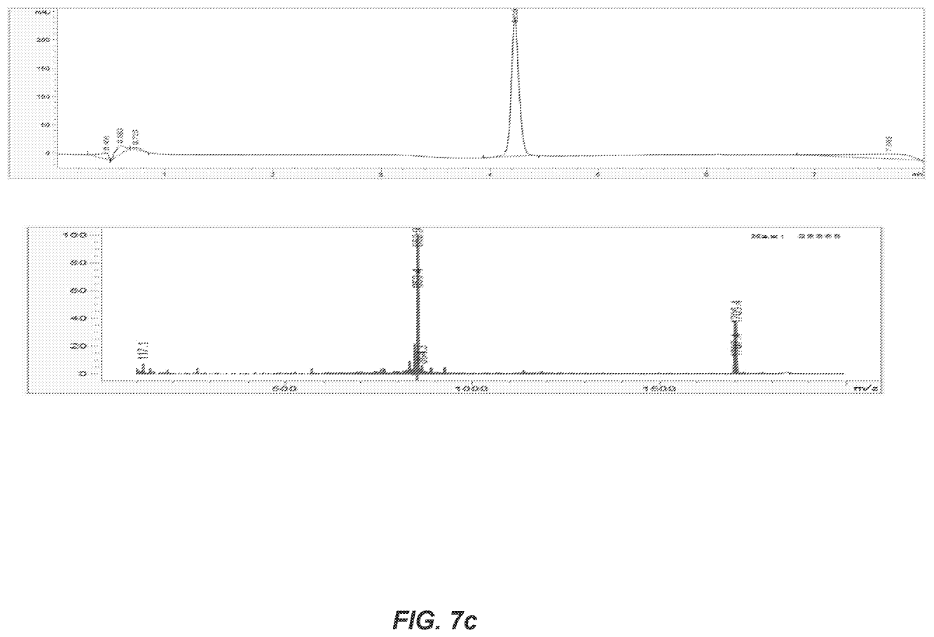

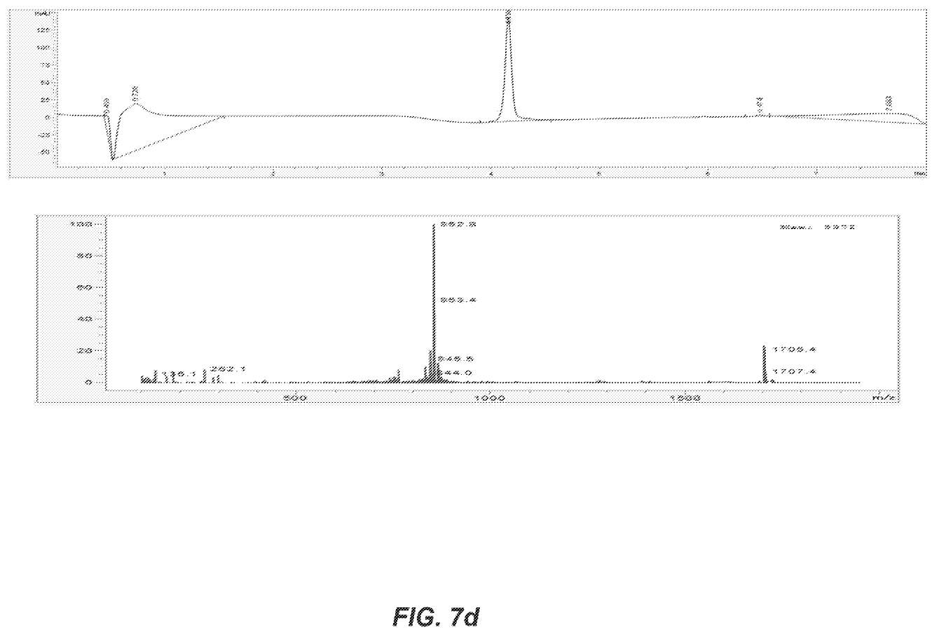

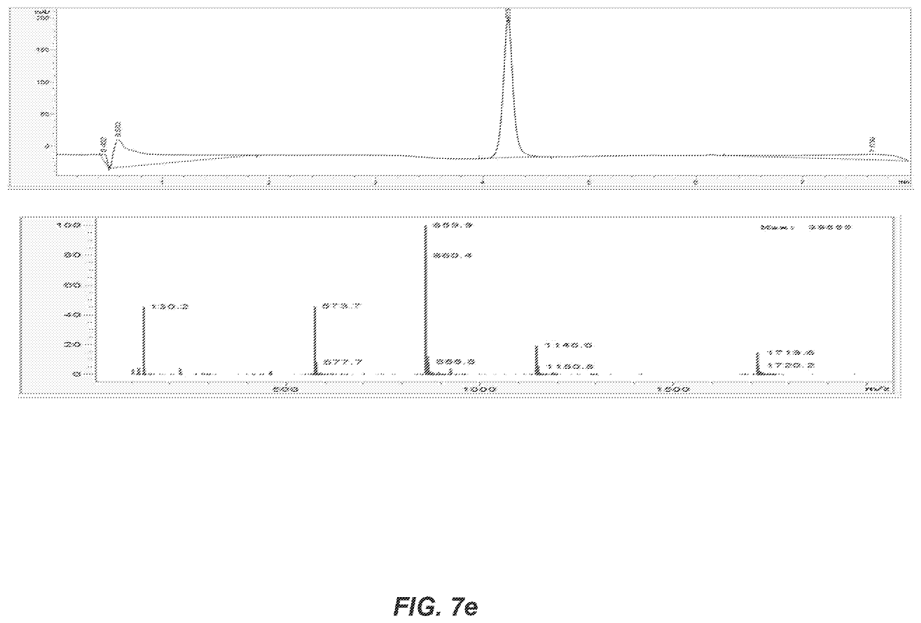

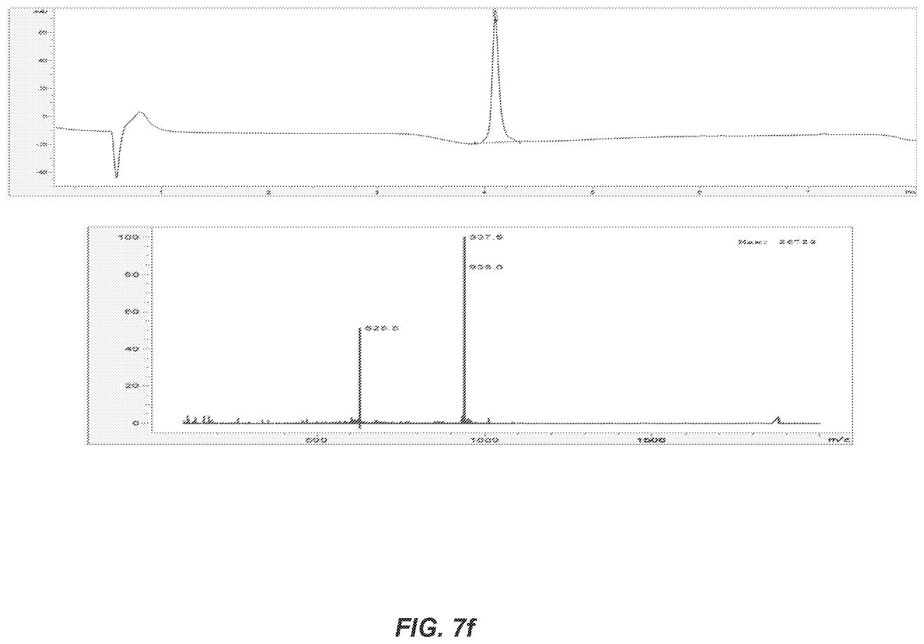

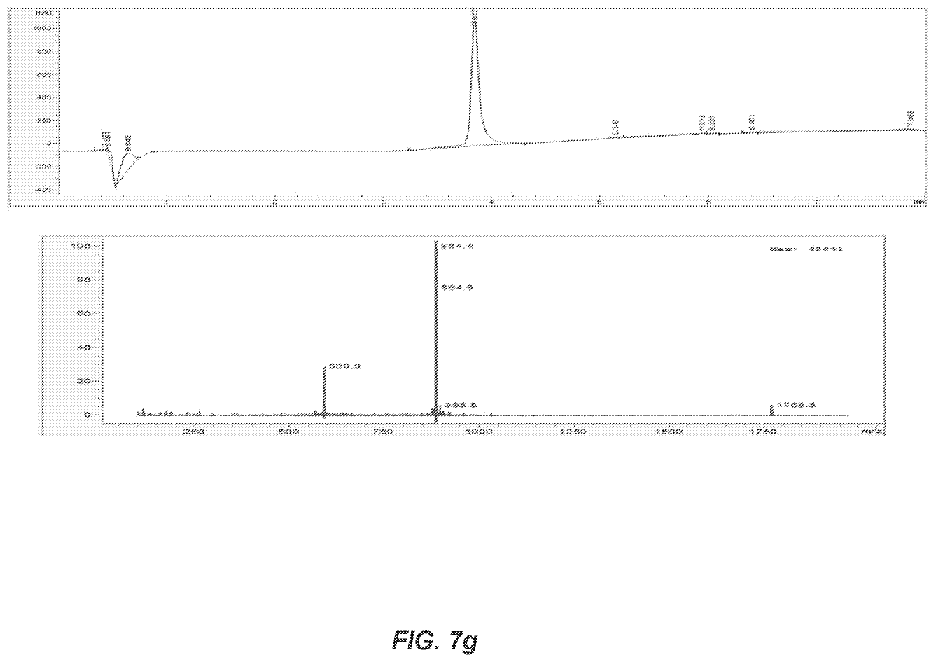

Strategies including dicarba, saturated dicarba, alkyne, thioether, ether, diselenide (Sec), and triazole bridge replacement have been attempted in CTxs modifications, as shown in FIG. 7a-C. However, such strategies lack broad applicability because of incompatibility of the mimetic moiety, reduced bioactivity, and toxicity. Head-to-tail backbone cyclization is another strategy to enhance peptide stability through prevention of degradation but, when applied RgIA, has resulted in potency drop on α9α10 nAChR binding affinity.

In one embodiment, the lactam linkage introduced in α-RgIA prevents degradation of the active globular conformation and suppresses disulfide scrambling. The NMR structure of the macrocyclic peptide overlays well with that of α-RgIA4, demonstrating that the cyclization does not perturb the overall conformation of backbone and side-chain residues. Finally, a molecular docking model can rationalize the selective binding between a macrocyclic analogue and the α9α10 nicotinic acetylcholine receptor. In vivo testing indicates that analogue 6 as discussed in the proceeding can prevent pain in a chemotherapy induced neuropathic pain model. Structurally, the introduced lactam bond can provide an additional conformational constraint to rigidify the bioactive conformation, suppress disulfide scrambling, and provide increases in human serum stability. These conformationally constrained antagonists are therefore promising candidates for antinociceptive therapeutic intervention.

In another embodiment, RgIA analogues can be stabilized by a disulfide surrogate, methylene thioacetal, to target human α9α10 nAChRs as non-opioid analgesic agents. Replacing disulfide loop I [Cys^{I}-Cys^{III}] with methylene thioacetal in the RgIA skeleton can result in a substantial potency loss whereas bridging loop II [Cys^{II}-Cys^{IV}] with methylene thioacetal can be accommodated and retain the analogue's bioactivity. One molecule, RgIA-5524, exhibits highly selective inhibition of human α9α10 nAChRs with an IC₅₀ of 0.9 nM. Moreover, RgIA-5524 showed greatly increased resistance to degradation in human serum over RgIA4. In vivo studies in mice showed that RgIA-5524 relieves chemotherapy-induced neuropathic pain. RgIA-5524 failed to alleviate neuropathic pain in α9 knockout mice demonstrating that α9-containing nAChRs are used for the observed therapeutic effects of RgIA-5524. Therefore, methylene thioacetal can be applied as a disulfide surrogate in conotoxin-based and other disulfide-rich peptide drug discovery.

There is an ongoing controversy regarding the therapeutic mechanism of action of α-CTxs, with a number of studies asserting that stimulation of GABAB receptors is used yet others indicating that blockade of α9 nAChRs is used. Studies disclosed herein show that wildtype and knockout (KO) mice demonstrate not only that a selective α9α10 nAChR antagonist is analgesic, but also that the presence of the α9-nAChR subunit is used for analgesic activity.

In one embodiment, an α-RgIA4 peptide analog can include a recognition finger region configured to bind to an α9α10 nicotinic acetylcholine receptor, and a side chain bonding configuration that protects an inter-cysteine sulfur linkage. The analog can have a binding affinity for the α9α10 nicotinic acetylcholine receptor that is at least 2.5% of a binding affinity of an α-RgIA4 peptide.

In another embodiment, an α-RgIA4 peptide analog can have a structure (e.g., globular) maintained by a protected inter-cysteine sulfur linkage. The structure (e.g., globular) can provide a binding affinity for the α9α10 nicotinic acetylcholine receptor that is at least 2.5% of a binding affinity of an α-RgIA4 peptide.

In yet another embodiment, an α-RgIA4 peptide analog can include a recognition finger region comprising D P R; and cystine residues comprising C^{I}, C^{II}, C^{III}, and C^{IV}. The cysteine residues C^{I} and C^{III} can be linked by a first inter-cysteine sulfur linkage, and the cysteine residues C^{II} and C^{IV} can be linked by a second inter-cysteine sulfur linkage. The second inter-cysteine sulfur linkage can be protected by a side chain bonding configuration.

In another embodiment, a method of maintaining an α-RgIA4 potency for an α9α10 nicotinic acetylcholine receptor in an α-RgIA4 analog can include protecting inter-cysteine sulfur linkages with a side chain bonding configuration that maintains a recognition finger region of the analog in an α-RgIA4 configuration (e.g., globular α-RgIA4 configuration).

In one more embodiment, a composition can include a combination of a therapeutically effective amount of the analog with a pharmaceutically acceptable carrier. In another embodiment, a method for treating in a subject, a condition that is responsive to α9α10 nicotinic acetylcholine receptor binding can include administering a therapeutically effective amount of the composition to the subject.

### Analogs of α-RgIA4

Side chain cyclization is one peptide stabilization method that may be applied. For example, a third cyclization bridge can be inserted at the termini through side chain cyclization to rigidify the active conformation of α-RgIA analogues while retaining binding activity, as shown in FIG. 1b-B. Conformationally constrained α-RgIA analogues with high potency, receptor selectivity, and enhanced serum stability can target the human α9α10 nicotinic acetylcholine receptor. Analog 6, as disclosed herein, demonstrates that the lactam linkage introduced in α-RgIA can prevent degradation of the active globular conformation and suppress disulfide scrambling.

In another stabilization method, by inserting a minimal functional carbon unit (CH₂) into disulfide, the unreducible methylene thioacetal can be an efficient disulfide surrogate. Replacing disulfide with methylene thioacetal can stabilize the globular active conformation of RgIA analogues. In one example, RgIA-5524 exhibited high potency (e.g., IC₅₀=0.9 nM) on human α9α10 nAChRs with high selectivity in comparison with other pain-related ion channels and receptors.

In one embodiment, an α-RgIA4 peptide analog can include a recognition finger region configured to bind to an α9α10 nicotinic acetylcholine receptor, and a side chain bonding configuration that protects an inter-cysteine sulfur linkage. The analog can have a binding affinity for the α9α10 nicotinic acetylcholine receptor that is at least 2.5% of a binding affinity of an α-RgIA4 peptide. In another embodiment, an α-RgIA4 peptide analog can have a structure (e.g., globular) maintained by a protected inter-cysteine sulfur linkage. The structure (e.g., globular) can provide a binding affinity for the α9α10 nicotinic acetylcholine receptor that is at least 2.5% of a binding affinity of an α-RgIA4 peptide.

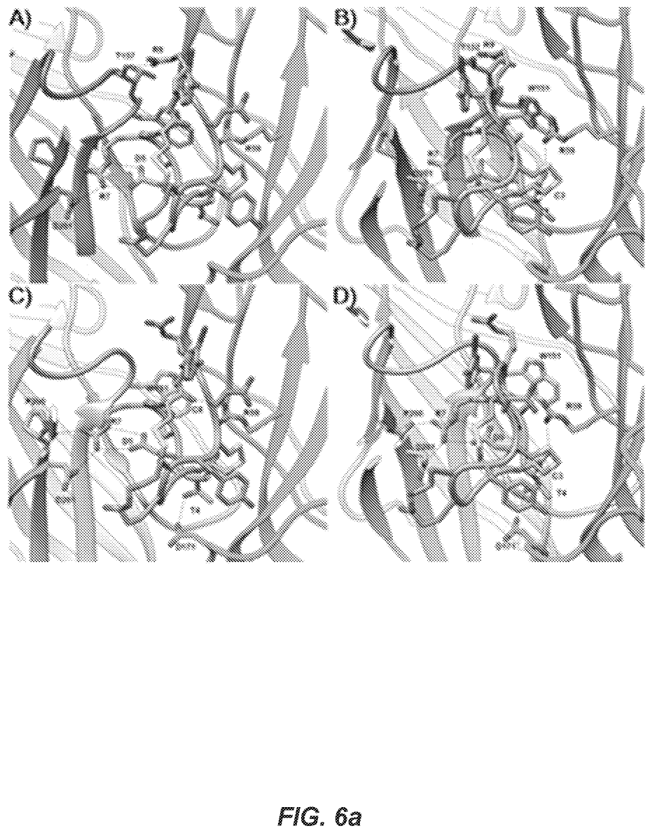

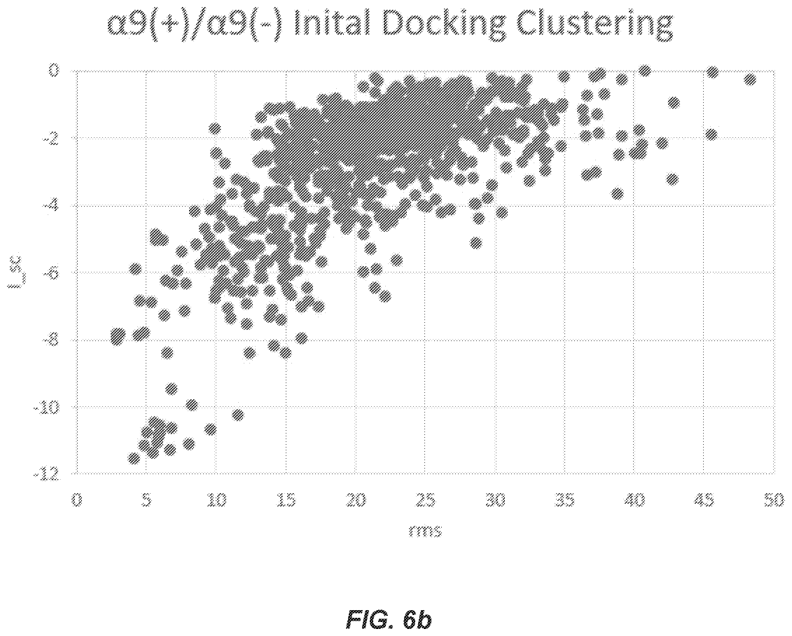

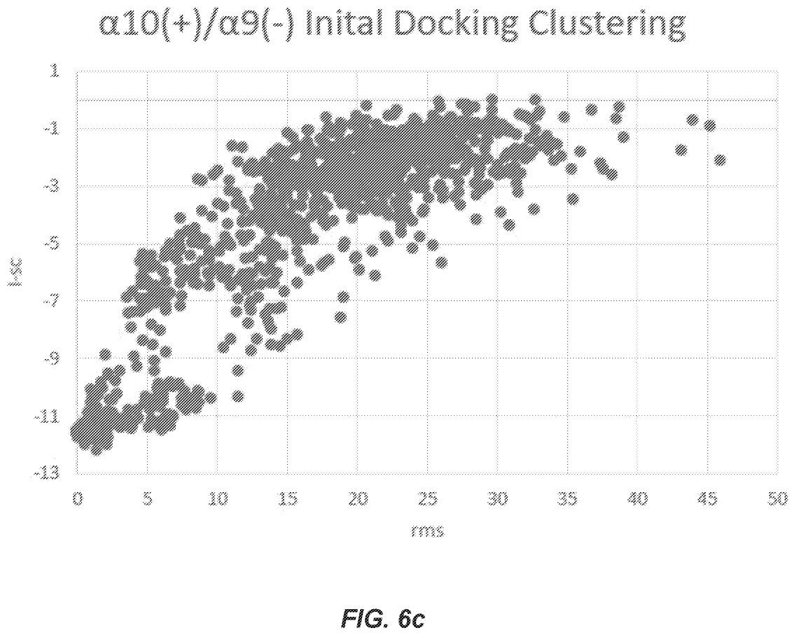

The recognition finger region can be configured to bind to an α9α10 nicotinic acetylcholine receptor, as modeled in FIG. 6a, and the α9 subunit, as modeled in FIG. 7a-B. The structure of the recognition finger region should be maintained to allow the binding to occur. One way of maintaining the structure of the recognition finger region (and therefore the binding affinity of the recognition finger region) can include protecting inter-cysteine linkages between the four cysteine residues found in an α-RgIA4 peptide analog, which can be numbered in the sequence order as C^{I}, C^{II}, C^{III}, and C^{IV}.

The inter-cysteine sulfur linkages can include direct sidechain linkages between sulfurs on each cysteine (e.g., a sulfur on C^{II} can be linked to a sulfur on C^{IV}) or an indirect linkage between sulfurs on each cysteine (e.g., a sulfur on C^{II} can be linked to a sulfur on C^{IV} via an intermediate such as carbon).

In one embodiment, an inter-cysteine sulfur linkage can be protected by a side chain bonding configuration. In one aspect, the side chain bonding configuration can comprise one or more of a methylene thioacetal, an N-terminal amino acid side chain that is cyclized to a C-terminal amino acid side chain with a lactam bridge, or a combination thereof.

When the side chain bonding configuration is a methylene thioacetal, the side chain bonding configuration can comprise an inter-cysteine linkage between C^{II} and C^{IV} in an α-RgIA4 peptide analog. Positioning the side chain bonding configuration (e.g., methylene thioacetal) in this position can stabilize the analog in a globular, active conformation without reducing the potency of the analog with respect to an α9α10 nicotinic acetylcholine receptor when compared to the α-RgIA4 peptide. On the other hand, positioning the side chain bonding configuration (e.g., methylene thioacetal) as an inter-cysteine linkage between C^{I} and C^{III} does not provide the enhanced potency with respect to an α9α10 nicotinic acetylcholine receptor when compared to the α-RgIA4 peptide.

Many of the analogues (e.g., Analogues 1 to 6) maintained the globular conformation of the α-RgIA4 peptide. However, one difference between the less potent RgIA-5617 and the more potent analogues (RgIA4, RgIA-5533, and 5524) was the Ca distances of the cysteine pairs. Both the Ca distance of the two cysteine pairs in RgIA-5617 were shortened (e.g., an average of 4.8 and 4.7 Å in cysteine loop I and loop II, respectively) compared to the other active molecules including RgIA4, RgIA-5533, and RgIA-5524 (e.g., an average of 5.4-6.1 A). In one example, the potency loss arising from methylene thioacetal replacement in loop-I (e.g., cys^{I}-cys^{III}) can cause a structural shrink that can reduce binding affinity to the α9α10 nicotinic acetylcholine receptor. In another example, the loop-I disulfide in alpha-CTxs can provide stacking interaction towards the α9α10 nicotinic acetylcholine receptor by directly contacting the C-loop disulfide of the α9(+) surface. Therefore, methylene thioacetal replacement at this loop can cause a reduction in potency compared to the more potent analogs by interfering with this binding site.

When the side chain bonding configuration is an N-terminal amino acid side chain that is cyclized to a C-terminal amino acid side chain with a lactam bridge, the N-terminal amino acid can be selected from the group consisting of glutamic acid and aspartic acid and the C-terminal amino acid can be selected from the group consisting of lysine, homo-lysine, ornithine, L-2,4-diaminobutyric acid, and L-2,3-diaminopropionic acid. In another example, the C-terminal amino acid can be selected from the group consisting of lysine and L-2,3-diaminopropionic acid. In one example, the N-terminal amino acid can be glutamic acid and the C-terminal amino acid can be lysine.

The side chain bonding configuration can provide the respective the α-RgIA4 analogue with an enhanced binding affinity for the α9α10 nicotinic acetylcholine receptor when compared to the α-RgIA4 peptide. For example, the analog can have a binding affinity for the α9α10 nicotinic acetylcholine receptor that is at least one or more of 2.5%, 5%, 7.5%, 15%, 25%, 40%, 50%, 80%, or substantially equal to a binding affinity of the α-RgIA4 peptide for the α9α10 nicotinic acetylcholine receptor. Additionally, the analog can have a binding affinity for the α9α10 nicotinic acetylcholine receptor that is greater than a binding affinity of an α-RgIA4 peptide for the α9α10 nicotinic acetylcholine receptor.

The side chain bonding configuration can not only provide the α-RgIA4 analogue with an enhanced binding affinity for the α9α10 nicotinic acetylcholine receptor but can also provide an increase in potency compared to a potency of an α-RgIA4 peptide. In one example, the analog can provide an α9α10 nicotinic acetylcholine receptor IC₅₀ value that is substantially equal to an α9α10 nicotinic acetylcholine receptor IC₅₀ value of the α-RgIA4 peptide. In another example, the analog can provide an α9α10 nicotinic acetylcholine receptor IC₅₀ value that is no greater than at least one or more of 2.0×, 3.0×, 5.0×, 15.0×, 25.0× the α9α10 nicotinic acetylcholine receptor IC₅₀ value of the α-RgIA4 peptide. The IC₅₀ value has a higher potency when the value is lower because a lower value indicates that a lower concentration can achieve the 50% threshold of inhibition.

In one aspect, the protected inter-cysteine linkage can be one or more of an inter-cysteine linkage (e.g. a side chain linkage) between C^{I} and C^{III}, C^{II} and C^{IV}, or a combination thereof. The protected inter-cysteine linkage (which can be protected by the side chain bonding configuration) can reduce one or more of disulfide bridge scrambling, disulfide bridge degradation, or a combination thereof as compared to an α-RgIA4 peptide, or α-RgIA4 peptide analog without a protected inter-cysteine sulfur linkage. Disulfide bridge scrambling can occur when disulfide bridges in a peptide degrade and then re-form in a different configuration. For example, in a first configuration, the α-RgIA4 peptide analog can have a first disulfide bridge between C^{I} and C^{III} and a second disulfide bridge between C^{II} and C^{IV}. After scrambling, there can be a first disulfide bride between C^{I} and C^{IV} and a second disulfide bridge between C^{II} and C^{III}. Disulfide bridge scrambling can result in a structural change in the peptide that can hinder the desired peptide function (e.g., inhibition of an α9α10 nicotinic acetylcholine receptor). Disulfide bridge degradation can occur when the disulfide bridges degrade without re-forming. This degradation can also result in a structural change in the peptide that can hinder the desired peptide function.

The side chain bonding configuration can also protect the inter-cysteine sulfur linkage to provide a stability for the α-RgIA4 peptide analog in human serum that is greater than the stability of an α-RgIA4 peptide in human serum. In one aspect, the stability in the human serum can be measured by the amount of peptide or peptide analog remaining after incubation of 0.1 mg/mL of the α-RgIA4 peptide analog or the α-RgIA4 peptide in 90% human serum AB type and incubated at 37° C. for at least one of 1, 2, 4, 8, or 24 hours.

In some examples, the stability in human serum of the α-RgIA4 peptide analog can be greater than at least one or more of 10%, 20%, 40%, 60%, 80%, 100%, 200%, 300%, 400%, 500%, or 1000% of the stability of the α-RgIA4 peptide in human serum. In another example, the stability in human serum of the α-RgIA4 peptide analog can be at least 50× greater than the stability of the α-RgIA4 peptide in human serum. In another example, the stability in human serum of the α-RgIA4 peptide analog can be at least 10× greater than the stability of the α-RgIA4 peptide in human serum. In another example, the stability in human serum of the α-RgIA4 peptide analog can be at least 2× greater than the stability of the α-RgIA4 peptide in human serum.

The stability of the α-RgIA4 peptide analog in comparison to the stability of the α-RgIA4 peptide can also be measured in reduced glutathione (GSH). In one example, the protected inter-cysteine sulfur linkage can provide a stability for the α-RgIA4 peptide analog in reduced glutathione that is greater than the stability of an α-RgIA4 peptide in reduced glutathione. In one example, the stability in the reduced glutathione can be measured by the amount of the α-RgIA4 peptide analog or the α-RgIA4 peptide remaining after incubation of 0.1 mg/mL of the α-RgIA4 peptide analog or the α-RgIA4 peptide in 10 equivalents of reduced glutathione in phosphate buffered saline (PBS) having a pH of 7.4 and incubated at 37° C. for at least one of 1, 2, 4, 8, or 24 hours.

In some examples, the stability in GSH of the α-RgIA4 peptide analog can be greater than at least one or more of 10%, 20%, 40%, 60%, 80%, 100%, 200%, 300%, 400%, 500%, 1000% compared to the stability of the α-RgIA4 peptide in GSH. In another example, the stability in GSH of the α-RgIA4 peptide analog can be at least 50× greater than the stability of the α-RgIA4 peptide in GSH. In another example, the stability in GSH of the α-RgIA4 peptide analog can be at least 10× greater than the stability of the α-RgIA4 peptide in GSH.

The selectivity of the α-RgIA4 peptide analog for the α9α10 nicotinic acetylcholine receptor can be compared to the selectivity of the α-RgIA4 peptide for the α9α10 nicotinic acetylcholine receptor. In one example, the protected inter-cysteine sulfur linkage can provide an α9α10 nicotinic acetylcholine receptor selectivity that is substantially equal to the α9α10 nicotinic acetylcholine receptor selectivity of an α-RgIA4 peptide. In another example, the protected inter-cysteine sulfur linkage can provide an α9α10 nicotinic acetylcholine receptor selectivity that is at least 100× more selective for the α9α10 nicotinic acetylcholine receptor compared to a selectivity of a different nicotinic acetylcholine receptor (nAChR) subtype. The different nAChR subtype can be selected from the group consisting of: α1β1δε, α2β2, α2β4, α3β2, α3β4 α4β2, α4β4, α6/α3β2β3, α6/α3β4, the like, or a combination thereof.

The safety profile of the α-RgIA4 peptide analog can also have a safety profile. In one aspect, the protected inter-cysteine sulfur linkage can provide a safety profile that is substantially equal to or greater than the safety profile of an α-RgIA4 peptide. The safety profile can be measured by one or more of: the analog present in a concentration of 100 μM inhibits less than 25% of the human ether-a-go-go-related gene (hERG) K⁺ channel as measured from an automated-whole cell patch-clamp assay; or the analog present in a concentration of 100 μM has inhibitory activity of less than about 20% as measured by a monoamine oxidase (MAO) assay; or the analog present in a concentration of 10 μM has inhibitory activity of less than 20% as measured in a CYP assay.

The side chain bonding configuration disclosed herein (e.g., inclusion of methylene acetal or linking side chains via a lactam bridge) can enhanced various aspects of the α-RgIA4 peptide analog. For example, the serum half-life of the α-RgIA4 peptide analog can be enhanced when compared to the serum half-life of the α-RgIA4 peptide. In another example, the circulation time of the α-RgIA4 peptide analog can be enhanced when compared to the circulation time of the α-RgIA4 peptide. In another example, the oral and/or buccal absorption of the α-RgIA4 peptide analog can be enhanced when compared to the oral and/or buccal absorption of the α-RgIA4 peptide. In another example, the bioavailability as measured by the AUC of the α-RgIA4 peptide analog can be enhanced when compared to the bioavailability as measured by the AUC of the α-RgIA4 peptide. In another example, the immunogenicity of the α-RgIA4 peptide analog can be enhanced when compared to the immunogenicity of the α-RgIA4 peptide.

In another example, the storage stability of the α-RgIA4 peptide analog can be enhanced when compared to the storage stability of the α-RgIA4 peptide. In one example, the storage stability can be measured when stored for a selected storage time at ambient humidity and temperature. In some cases, a storage time of greater than one or more of 1 day, 1 week, 2 weeks, 4 weeks, 3 months, 6 months, one year, or combinations thereof can be measured to compare enhancements in stability between the α-RgIA4 peptide analog and the α-RgIA4 peptide.

In yet another embodiment, an α-RgIA4 peptide analog can include a recognition finger region comprising D P R; and cystine residues comprising C^{I}, C^{II}, C^{III}, and C^{IV}. The cysteine residues C^{I} and C^{III} can be linked by a first inter-cysteine sulfur linkage, and the cysteine residues C^{II} and C^{IV} can be linked by a second inter-cysteine sulfur linkage. The second inter-cysteine sulfur linkage can be protected by a side chain bonding configuration. In one aspect, second inter-cysteine sulfur linkage can comprise methylene thioacetal, an N-terminal amino acid side chain is cyclized to a C-terminal amino acid side chain with a lactam bridge, or a combination thereof.

In one aspect, when the second inter-cysteine sulfur linkage comprises methylene thioacetal, the analog can comprise the amino acid sequence Xaa₁ C C Xaa₂ D P R C Xaa₃ Xaa₄ Xaa₅ C Xaa₆ wherein Xaa₁₋₆ is any amino acid other than C.

In one example, when the analog comprises the amino acid sequence Xaa₁ C C Xaa₂ D P R C Xaa₃ Xaa₄ Xaa₅ C Xaa₆, then: Xaa₁ can be any proteinogenic or non-proteinogenic amino acid other than C; Xaa₂ can be any proteinogenic or non-proteinogenic amino acid other than C; Xaa₃ can be a member selected from the group consisting of: (Cit) or any proteinogenic or non-proteinogenic positive amino acid; Xaa₄ can be any proteinogenic or non-proteinogenic aromatic amino acid; Xaa₅ can be any proteinogenic or non-proteinogenic positive amino acid; and Xaa₆ can be any proteinogenic or non-proteinogenic aromatic amino acid.

In another aspect, when the second inter-cysteine sulfur linkage comprises methylene thioacetal, the analog can comprise the amino acid sequence Xaa₁ C C Xaa₂ D P R C Xaa₃ Xaa₄ Xaa₅ C Xaa₆ Xaa₇ wherein Xaa₁₋₇ is any amino acid other than C.

In another example, when the analog comprises the amino acid sequence Xaa₁ C C Xaa₂ D P R C Xaa₃ Xaa₄ Xaa₅ C Xaa₆ Xaa₇, then: Xaa₁ can be any proteinogenic or non-proteinogenic amino acid other than C, Xaa₂ is any proteinogenic or non-proteinogenic amino acid other than C, Xaa₃ can be a member selected from the group consisting of: (Cit) or any proteinogenic or non-proteinogenic positive amino acid, Xaa₄ can be any proteinogenic or non-proteinogenic aromatic amino acid, Xaa₅ can be any proteinogenic or non-proteinogenic positive amino acid, Xaa₆ can be any proteinogenic or non-proteinogenic aromatic amino acid, and Xaa₇ can be any proteinogenic or non-proteinogenic amino acid other than C.

In another aspect, when the second inter-cysteine sulfur linkage comprises methylene thioacetal, the analog can comprise the amino acid sequence G C C T D P R C Xaa₃ Xaa₄ Q C Xaa₆, wherein Xaa₁ is G, Xaa₂ is T, Xaa₅ is Q, and Xaa_{3, 4, or 6} is any amino acid other than C.

In another aspect, when the second inter-cysteine sulfur linkage comprises methylene thioacetal, the analog can comprise the amino acid sequence G C C T D P R C Xaa₃ Xaa₄ Q C Xaa₆, wherein: Xaa₃ is a member selected from the group consisting of (Cit) and R, Xaa₄ is a member selected from the group consisting of (iY) and Y, and Xaa₆ is a member selected from the group consisting of (bhY), Y, and bA.

In another aspect, when the second inter-cysteine sulfur linkage comprises methylene thioacetal, the analog can comprise the amino acid sequence G C C T D P R C (Cit) (iY) Q C Y (SEQ ID NO: 10), wherein: Xaa₃ is (Cit), Xaa₄ is (iY), and Xaa₆ is Y.

In another aspect, when the second inter-cysteine sulfur linkage comprises methylene thioacetal, the analog can comprise the amino acid sequence G C C T D P R C Xaa₃ Xaa₄ Q C Xaa₆ Xaa₇, wherein Xaa₁ is G, Xaa₂ is T, Xaa₅ is Q, and Xaa_{3, 4, 6, or 7} is any amino acid other than C.

In another aspect, when the second inter-cysteine sulfur linkage comprises methylene thioacetal, the analog can comprise the amino acid sequence G C C T D P R C Xaa₃ Xaa₄ Q C Xaa₆ Xaa₇, wherein: Xaa₃ is a member selected from the group consisting of (Cit) and R, Xaa₄ is a member selected from the group consisting of (iY) and Y, Xaa₆ is a member selected from the group consisting of (bhY), Y, and bA, and Xaa₇ is R.

In another aspect, when the second inter-cysteine sulfur linkage comprises methylene thioacetal, the analog can comprise the amino acid sequence G C C T D P R C R (iY) Q C (bhY) R (SEQ ID NO: 12), wherein: Xaa₃ is R, Xaa₄ is (iY), and Xaa₆ is (bhY).

In another aspect, when the second inter-cysteine sulfur linkage comprises methylene thioacetal, the analog can comprise the amino acid sequence G C C T D P R C R (iY) Q C (bA) R (SEQ ID NO: 13), wherein: Xaa₃ is R, Xaa₄ is (iY), and Xaa₆ is (bA).

In another aspect, the N-terminal amino acid side chain can be cyclized to a C-terminal amino acid side chain with a lactam bridge. When the N-terminal amino acid side chain can be cyclized to a C-terminal amino acid side chain with a lactam bridge, the N-terminal amino acid can be selected from the group consisting of glutamic acid and aspartic acid. In another aspect, the C-terminal amino acid can be selected from the group consisting of lysine and L-2,3-diaminopropionic acid. In another example, the C-terminal amino acid can be selected from the group consisting of lysine, homo-lysine, ornithine, L-2,4-diaminobutyric acid, and L-2,3-diaminopropionic acid. In another aspect, the N-terminal amino acid can be glutamic acid and the C-terminal amino acid is lysine.

In another aspect, when the N-terminal amino acid side chain is cyclized to a C-terminal amino acid side chain with a lactam bridge, the analog can comprise the amino acid sequence Xaa₈ Xaa₉ C C Xaa₁₀ D P R C Xaa₁₁ Xaa₁₂ Xaa₁₃ C Xaa₁₄ Xaa₁₅, wherein Xaa₈₋₁₅ is any amino acid other than C.

In another aspect, when the N-terminal amino acid side chain is cyclized to a C-terminal amino acid side chain with a lactam bridge, the analog can comprise the amino acid sequence Xaa₈ Xaa₉ C C Xaa₁₀ D P R C Xaa₁₁ Xaa₁₂ Xaa₁₃ C Xaa₁₄ Xaa₁₅, wherein: Xaa₈ is a member selected from the group consisting of E and D, Xaa₁₅ is a member selected from the group consisting of K and (Dap), and Xaa₉₋₁₄ is any amino acid other than C.

In another aspect, when the N-terminal amino acid side chain is cyclized to a C-terminal amino acid side chain with a lactam bridge, the analog can comprise the amino acid sequence Xaa₈ Xaa₉ C C T D P R C Xaa₁₁ Xaa₁₂ Q C Y Xaa₁₅, wherein: Xaa₈ is a member selected from the group consisting of E and D, Xaa₁₀ is T, Xaa₁₃ is Q, Xaa₁₄ is Y, Xaa₁₅ is a member selected from the group consisting of K and (Dap), and Xaa_{9, 11, or 12} is any amino acid other than C.

In another aspect, when the N-terminal amino acid side chain is cyclized to a C-terminal amino acid side chain with a lactam bridge, the analog can comprise the amino acid sequence Xaa₈ Xaa₉ C C T D P R C Xaa₁₁ Xaa₁₂ Q C Y Xaa₁₅, wherein: Xaa₈ is a member selected from the group consisting of E and D, Xaa₉ is G or (bA), Xaa₁₁ is R or (Cit), Xaa₁₂ is Y or (iY), and Xaa₁₅ is a member selected from the group consisting of K and (Dap).

In another aspect, when the N-terminal amino acid side chain is cyclized to a C-terminal amino acid side chain with a lactam bridge, the analog can comprise the amino acid sequence E G C C T D P R C (Cit) Y Q C Y K (SEQ ID NO: 5), wherein: Xaa₈ is E, Xaa₉ is G, Xaa₁₁ is (Cit), Xaa₁₂ is Y, and Xaa₁₅ is K.

In another aspect, when the N-terminal amino acid side chain is cyclized to a C-terminal amino acid side chain with a lactam bridge, the analog can comprise the amino acid sequence E (bA) C C T D P R C (Cit) Y Q C Y K (SEQ ID NO: 6), wherein: Xaa₈ is E, Xaa₉ is (bA), Xaa₁₁ is (Cit), Xaa₁₂ is Y, and Xaa₁₅ is K.

In another aspect, when the N-terminal amino acid side chain is cyclized to a C-terminal amino acid side chain with a lactam bridge, the analog can comprise the amino acid sequence E G C C T D P R C (Cit) (iY) Q C Y K (SEQ ID NO: 7, wherein: Xaa₈ is E, Xaa₉ is G, Xaa₁₁ is (Cit), Xaa₁₂ is (iY), and Xaa₁₅ is K.

In another aspect, when the N-terminal amino acid side chain is cyclized to a C-terminal amino acid side chain with a lactam bridge, the analog can comprise the amino acid sequence E G C C T D P R C R (iY) Q C Y K (SEQ ID NO: 8), wherein: Xaa₈ is E, Xaa₉ is G, Xaa₁₁ is R, Xaa₁₂ is (iY), and Xaa₁₅ is K.

In another embodiment, a method of maintaining an α-RgIA4 potency for an α9α10 nicotinic acetylcholine receptor in an α-RgIA4 analog can include protecting inter-cysteine sulfur linkages with a side chain bonding configuration that maintains a recognition finger region of the analog in an α-RgIA4 configuration (e.g., globular α-RgIA4 configuration).

In one aspect, the analog can bind to the α9α10 nicotinic acetylcholine receptor with an affinity that is: at least one or more of 2.5%, 5%, 7.5%, 15%, 25%, 40%, 50%, 80%, or substantially equal to a binding affinity of the α-RgIA4 peptide, or greater than a binding affinity of an α-RgIA4 peptide.

In another aspect, the analog can inhibit the α9α10 nicotinic acetylcholine receptor with an IC₅₀ value that is: substantially equal to an α9α10 nicotinic acetylcholine receptor IC₅₀ value of the α-RgIA4 peptide, or no greater than at least one or more of 2.0×, 3.0×, 5.0×, 15.0×, 25.0× the α9α10 nicotinic acetylcholine receptor IC₅₀ value of the α-RgIA4 peptide.

In another aspect, protecting the inter-cysteine sulfur linkage can provide a α9α10 nicotinic acetylcholine receptor (nAChR) selectivity that is at least one or more of 2×, 5×, 10×, 20×, 30×, 40×, 50×, 75×, 100×, 150×, or 200× more selective for the α9α10 nicotinic acetylcholine receptor compared to a selectivity of a different nAChR subtype. In another aspect, protecting the inter-cysteine sulfur linkage can provide a stability in human serum of the α-RgIA4 peptide analog of at least 100× greater than the stability in human serum of an α-RgIA4 peptide.

In another aspect, protecting the inter-cysteine linkage can comprise protecting one or more of an inter-cysteine linkage between C^{I} and C^{III}, C^{II} and C^{IV}, or a combination thereof. In one example, protecting the inter-cysteine sulfur linkages can comprise inserting a methylene thioacetal between C^{II} and C^{IV}. In another example, protecting the inter-cysteine sulfur linkages can comprise creating a lactam bridge between an N-terminal amino acid and a C-terminal amino acid.

### Methods of Preparing α-RgIA4 Analogues

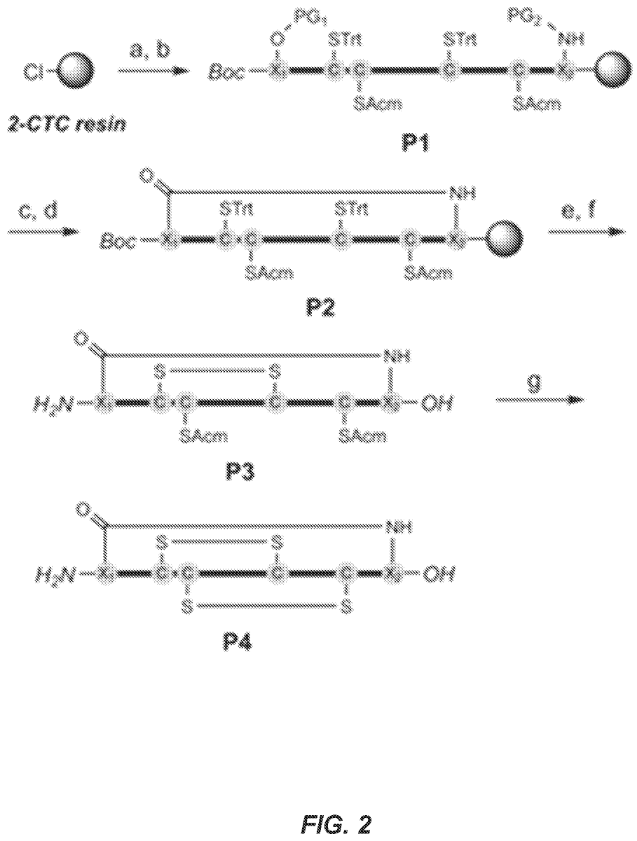

In one embodiment, an active cyclic α-RgIA4 analogue can be prepared as shown in FIG. 2. The newly introduced lactam bridge can be synthesized on resin followed by a two-operation liquid phase oxidation process in which a regioselective disulfide-bond arrangement can be applied to maintain the globular isomer. In detail, side-chain protected P1 can be synthesized through automated 9-fluorenylmethyloxycarbonyl (Fmoc) solid-phase peptide synthesis (SPPS) on 2-chlorotrityl chloride (2-CTC) resin with N-terminal Fmoc removal and re-protected with tert-butyloxycarbonyl (Boc). The terminal side chain amine and acid can be orthogonally deprotected (e.g., protecting group 1 (PG₁) and protecting group 2 (PG₂)) and further cyclized to form the lactam bridged molecule complex P2. The lactam cyclized peptide can be generated through cleavage, purification, and can undergo air oxidation to create the bicyclic product P3. Finally, fully folded P4 can be generated through an in situ iodine oxidative deprotection-disulfide formation.

In another embodiment, an active methylene thioacetal α-RgIA4 analogue can be prepared as shown in FIG. 7a-D. The chemical synthesis of RgIA analogues can be achieved by using 9-fluorenylmethyloxycarbonyl (Fmoc) solid-phase peptide synthesis (SPPS) on 2-chlorotrityl chloride (2-CTC) resin followed by a two-operation and regioselective intramolecular bond formation reactions. The correct scaffold folding is Cys^{I}-Cys^{III}, Cys^{II}-Cys^{IV} or its corresponding methylene thioacetal replacement with the same connectivity. Bonds were explicitly formed in an order of 1) methylene thioacetal formation on free Cys after trityl (Trt) removal through cleavage, 2) disulfide bond formation via in situ oxidative acetamidomethyl (Acm) deprotection-coupling process, and 3) repeating methylene thioacetal formation to generate the bis-methylene thioacetal replaced analogue.

In detail, after cleavage of the assembled peptide chain from 2-CTC resin, the Trt protections can be removed and the target methylene thioacetal bond can be formed by treatment with diiodomethane in the presence of tris(2-carboxyethyl)phosphine hydrochloride (TCEPHCl), potassium carbonate and trimethylamine (Et₃N). This conversion can be conducted in as large as 300 mg scale in one batch, which can enable a large preparation of target peptides. The second disulfide bridge can be formed after Acm deprotection by the treatment of excess iodine in 25% aqueous acetic acid (AcOH) to yield fully folded peptides.

### Compositions and Dosage Forms

With this in mind, in one more embodiment, a composition can include a combination of a therapeutically effective amount of an analog disclosed herein with a pharmaceutically acceptable carrier.

In one aspect, the analog can be present at a concentration of from about 0.0001 wt % to about 10 wt %. In one example, the analog can be present in the composition at a concentration of from about 0.0001 wt % to about 1 wt %. In another example, the analog can be present in the composition at a concentration of from about 0.001 wt % to about 1 wt %. In one more example, the analog can be present in the composition at a concentration of from about 0.01 wt % to about 0.1 wt %. In some examples, the analog can be present in the composition at a concentration of from about 0.005 wt % to about 0.05 wt %.

In one aspect, the pharmaceutically acceptable carrier can include one or more of water, a tonicity agent, a buffering agent, a preservative, the like, or a combination thereof.

In some examples, the carrier can include a tonicity agent. Non-limiting examples of tonicity agents can include sodium chloride, potassium chloride, calcium chloride, magnesium chloride, mannitol, sorbitol, dextrose, glycerin, propylene glycol, ethanol, trehalose, phosphate-buffered saline (PBS), Dulbecco's PBS, Alsever's solution, Tris-buffered saline (TBS), water, balanced salt solutions (BSS), such as Hank's BSS, Earle's BSS, Grey's BSS, Puck's BSS, Simm's BSS, Tyrode's BSS, and BSS Plus, the like, or combinations thereof. The tonicity agent can be used to provide an appropriate tonicity of the composition. In one aspect, the tonicity of the composition can be from about 250 to about 350 milliosmoles/liter (mOsm/L). In another aspect, the tonicity of the composition can be from about 277 to about 310 mOsm/L.

In some examples, the carrier can include a pH adjuster or buffering agent. Non-limiting examples of pH adjusters or buffering agents can include a number of acids, bases, and combinations thereof, such as hydrochloric acid, phosphoric acid, citric acid, sodium hydroxide, potassium hydroxide, calcium hydroxide, acetate buffers, citrate buffers, tartrate buffers, phosphate buffers, triethanolamine (TRIS) buffers, the like, or combinations thereof. Typically, the pH of the therapeutic composition can be from about 5 to about 9, or from about 6 to about 8. In another example, the pH of the therapeutic composition can be from about 5 to about 6.

In some examples, the carrier can include a preservative. Non-limiting examples of preservatives can include ascorbic acid, acetylcysteine, bisulfite, metabisulfite, monothioglycerol, phenol, meta-cresol, benzyl alcohol, methyl paraben, propyl paraben, butyl paraben, benzalkonium chloride, benzethonium chloride, butylated hydroxyl toluene, myristyl gamma-picolimium chloride, 2-phenoxyethanol, phenyl mercuric nitrate, chlorobutanol, thimerosal, tocopherols, the like, or combinations thereof.

In one aspect, the composition can further comprise an additional active agent. In one aspect, the additional active agent is a member selected from the group consisting of: an anti-inflammatory agent, an anesthetic, a secondary analgesic peptide, a non-peptide analgesic, the like, or a combination thereof.

In one example, the additional active agent can be an anti-inflammatory agent. Non-limiting examples of anti-inflammatory agents can include ibuprofen, naproxen, aspirin, diclofenac, celecoxib, sulindac, oxaprozin, piroxicam, indomethacin, meloxicam, fenoprofen, difunisal, etodolac, ketorolac, meclofenamate, nabumetone, salsalate, ketoprofen, tolmetin, flurbiprofen, mefenamic acid, famotidine, bromfenac, nepafenac, prednisone, cortisone, hydrocortisone, methylprednisolone, deflazacort, prednisolone, fludrocortisone, amcinonide, betamethasone diproprionate, clobetasol, clocortolone, dexamethasone, diflorasone, durasteride, flumethasone pivalate, flunisolide, fluocinolone acetonide, fluocinonide, fluorometholone, fluticasone propionate, flurandrenolide, hydroflumethiazide, the like, hydrates thereof, acids thereof, bases thereof, or salts thereof, or combinations thereof.

In one example, the additional active agent can be an anesthetic. Non-limiting examples of anesthetics can include articaine, bupivacaine, cinchocaine, etidocaine, levobupivacaine, lidocaine, mepivacaine, prilocaine, ropivacaine, trimecaine, the like, or combinations thereof.

In one example, the additional active agent can be a secondary analgesic peptide. In one example, the additional active agent can be a non-peptide analgesic. Non-limiting examples of non-peptide analgesics can include acetaminophen, codeine, dihydrocodeine, tramadol, meperidine, hydrocodone, oxycodone, morphine, fentanyl, hydromorphone, buprenorphine, methadone, diamorphine, pethidine, the like, hydrates thereof, acids thereof, bases thereof, or salts thereof, or combinations thereof.

In one aspect, the additional active agent can be present at a concentration of from about 0.0001 wt % to about 10 wt %. In one example, the additional active agent can be present in the composition at a concentration of from about 0.0001 wt % to about 1 wt %. In another example, the additional active agent can be present in the composition at a concentration of from about 0.001 wt % to about 1 wt %. In one more example, the additional active agent can be present in the composition at a concentration of from about 0.01 wt % to about 0.1 wt %. In some examples, the additional active agent can be present in the composition at a concentration of from about 0.005 wt % to about 0.05 wt %.

In another aspect, the composition can be formulated as one of: a solution, a suspension, an emulsion, a gel, a hydrogel, a thermo-responsive gel, a cream, an ointment, a paste, an adhesive, a liquid reservoir, a patch, or a combination thereof. In some aspects, the composition can be suitable for topical, transdermal, intravenous, subcutaneous administration, the like, or a combination thereof. In one aspect, the composition can be suitable for subcutaneous injection.

### Methods of Treatment

In yet another embodiment, a method for treating in a subject, a condition that is responsive to α9α10 nicotinic acetylcholine receptor binding can include administering a therapeutically effective amount of the composition to the subject. In one aspect, the condition can be pain. In another aspect, the condition can be spinal polyradiculopathy. In another aspect, the condition can be postherpetic neuralgia. In another aspect, the condition can be trigeminal neuralgia. In another aspect, the condition can be complex regional pain syndrome. In another aspect, the condition can be multiple sclerosis.

When the condition is pain, the pain can be neuropathic pain including one or more of: chemo-induced neuropathy (CIPN), diabetic neuropathy, arthritic neuropathy, osteoarthritic neuropathy, the like, or a combination thereof. In another aspect, the pain can be HIV pain. In another aspect, the pain can be pain associated with leprosy. In another aspect, the pain can be one or more of post-surgical pain, post-traumatic pain, the like, or a combination thereof.

In another aspect, the condition can be cancer. The cancer can include one or more of epithelial cancer, lung cancer, breast cancer, the like, or a combination thereof.

In another aspect, the condition can be inflammation. In one aspect, the inflammation can be mediated by immune cells, associated with rheumatism, the like, or a combination thereof. Illustrative inflammatory conditions that can be treated include inflammation, chronic inflammation, rheumatic diseases (including arthritis, lupus, ankylosing spondylitis, fibromyalgia, tendonitis, bursitis, scleroderma, and gout), sepsis, fibromyalgia, inflammatory bowel disease (including ulcerative colitis and Crohn's disease), sarcoidosis, endometriosis, uterine fibroids, inflammatory skin diseases (including psoriasis and impaired wound healing), inflammatory conditions of the lungs (including asthma and chronic obstructive pulmonary disease), diseases associated with inflammation of the nervous system (including multiple sclerosis, Parkinson's disease and Alzheimer's disease), periodontal disease, and cardiovascular disease.

In one aspect, the composition can be a dosage form having from about 25 μl to about 1 ml of the α-RgIA4 analog. In another aspect, the composition can be a dosage form having from about 1 ml to about 5 ml of the α-RgIA4 analog. In one aspect, the composition can be a dosage form having from about 5 ml to about 10 ml of the α-RgIA4 analog.

In another embodiment, treatment can provide a reduction in symptoms within a selected amount of time after administration. Administering the therapeutically effective amount of the topical composition can reduce the symptoms associated with the condition. In another aspect, the treatment can provide a reduction in symptoms of at least 10% within a selected amount of time after administration. In one example, the treatment can provide a reduction in symptoms of at least 20% within a selected amount of time after administration. In one more example, the treatment can provide a reduction in symptoms of at least 30% within a selected amount of time after administration. In yet another example, the treatment can provide a reduction in symptoms of at least 50% within a selected amount of time after administration.

The selected time after administration that achieves the reduction in symptoms can vary. In one example, the selected amount of time can be less than 15 seconds after administration. In another example, the selected amount of time can be less than 30 seconds after administration. In another example, the selected amount of time can be less than 60 seconds after administration. In another example, the selected amount of time can be less than 5 minutes after administration. In another example, the selected amount of time can be less than 15 minutes after administration. In another example, the selected amount of time can be less than 30 minutes after administration.

In yet another aspect, the therapeutically effective amount of the composition can be administered to the subject 1 to 10 times per day. In one example, the composition can be administered to the subject 1 to 10 times per day. In another example, the composition can be administered to the subject 1 to 5 times per day. In yet another example, the composition can be administered to the subject 3 to 5 times per day.

In one more aspect, the therapeutically effective amount of the composition can be administered to the subject according to a dosage regimen. In one example, the composition can be administered at least once per day for a duration of from about a single day to about 12 months. In another example, the composition can be administered at least once per day for a duration of from about a single day to about 6 months. In one more example, the composition can be administered at least once per day for a duration of from about a single day to about 3 months. In yet another example, the composition can be administered at least once per day for a duration of from about a single day to about 1 month.

In another aspect, administering the therapeutically effective amount of the composition can be as a subcutaneous dosage form, a transdermal dosage form, a topical dosage form, an intravenous dosage form, the like, or a combination thereof.

In another aspect, a composition for use in the treatment of a condition in a subject that is responsive to α9α10 nicotinic acetylcholine receptor binding can comprise a therapeutically effective amount of the composition. In another aspect, the use of a composition in the manufacture of a medicament for the treatment of a condition in a subject that is responsive to α9α10 nicotinic acetylcholine receptor binding can comprise a therapeutically effective amount of the composition.

### Sequence Listing

Table 1 sets forth the sequences for RgIA, RgIA4 and RgIA4 analogues.

TABLE 1

| Peptide | SEQUENCE | SEQ ID NO. |
|---|---|---|
| RgIA | G C C S D P R C R Y R C R | 1 |
| RgIA4 | G C C T D P R C (Cit) (iY) Q C Y | 2 |
| | [X8 X9 C C X10 D P R C X11 | 3 |
| | X12 X13 C X14 X15] | |
| | [X8 X9 C C X10 D P R C | 4 |
| | X11 X12 X13 C X14 X15] | |
| | [X8 X9 C C T D P R C X11 X12 Q C Y X15] | 5 |
| | [X8 X9 CC T D P R C X11 X12 Q C Y X15] | 6 |
| Analogue 1 | [D G C C T D P R C (Cit) Y Q C Y (Dap)] | 7 |
| Analogue 2 | [D G C C T D P R C (Cit) Y Q C Y K] | 8 |
| Analogue 3 | [E G C CT D P R C (Cit) Y Q C Y K] | 9 |
| Analogue 4 | [E (^{b}A) C CT D PR C (Cit) Y Q C Y K] | 10 |
| Analogue 5 | [E G C C T D P R C (Cit) (iY) Q C Y K] | 11 |
| Analogue 6 | [E G C C T D P R C R (iY) Q C Y K] | 12 |
| ²⁴ | X1 C^{I} C^{II} X2 D P R C^{III} X3 X4 X5 C^{IV} X6 | 13 |
| ²⁴ | X1 C^{I} C^{II} X2 D P R C^{III} X3 X4 X5 C^{IV} X6 | 14 |
| ²⁴ | G C^{I} C^{II} T D P R C^{III} X3 X4 QC^{IV} X6 | 15 |
| ²⁴ | G C^{I} C^{II} T D P R C^{III} X3 X4 Q C^{IV} X6 | 16 |
| RgIA-5617¹³ | G C^{I} C^{II} T D P R C^{III} (Cit) (iY) Q C^{IV} Y | 17 |
| RgIA-5533²⁴ | G C^{I} C^{II} T D P R C^{III} (Cit) (iY) Q C^{IV} Y | 18 |
| RgIA-5618^{13,24} | G C^{I} C^{II} T D P R C^{III} (Cit) (iY) Q C^{IV} Y | 19 |
| ²⁴ | X1 C^{I} C^{II} X2 D PR C^{III} X3 X4 X5 C^{IV} X6 X7 | 20 |
| ²⁴ | X1 C^{I} C^{II} X2 D PR C^{III} X3 X4 X5 C^{IV} X6 X7 | 21 |
| ²⁴ | G C^{I} C^{II} T D P RC^{III} X3 X4 Q C^{IV} X6 X7 | 22 |
| ²⁴ | G C^{I} C^{II} T D P R C^{III} X3 X4 Q C^{IV} X6 R | 23 |
| RgIA-5524²⁴ | G C^{I} C^{II} T D P R C^{III} R (iY) Q C^{IV} (bhY) R | 24 |
| RgIA-5573²⁴ | G C^{I} C^{II} T D P R C^{III} R (iY) Q C^{IV} (bA) R | 25 |

Note 1:
[] refers to a peptide that is cyclized by a lactam bridge between the side chains of the amino acid in the first position (e.g., D or E) and the side chains of the amino acid in the final position (e.g., Dap or K).

Note 2: C^{I}, C^{II}, C^{III}, and C^{IV} refer to the order of the cysteines in the peptide in relation to the N-terminus. E.g., C^{I} is closer to the N-terminus than C^{II}, C^{II} is closer to the N-terminus than C^{III}, and C^{III} is closer to the N-terminus than C^{IV}.
Note 3: X1-X15 refer to Xaa as recited herein.
¹³ refers to methylene thioacetal in the inter-cysteine sulfur linkage of C^{I} and C^{III}.
²⁴ refers to methylene thioacetal in the inter-cysteine sulfur linkage of C^{II} and C^{IV}.

### Examples

In one example, an α-RgIA4 peptide analog can comprise: a recognition finger region configured to bind to an α9α10 nicotinic acetylcholine receptor; and a side chain bonding configuration that protects an inter-cysteine sulfur linkage, wherein the analog has a binding affinity for the α9α10 nicotinic acetylcholine receptor that is at least 2.5% of a binding affinity of an α-RgIA4 peptide.

In another example, an α-RgIA4 peptide analog can have a structure maintained by a protected inter-cysteine sulfur linkage, said structure providing a binding affinity for the α9α10 nicotinic acetylcholine receptor that is at least 2.5% of a binding affinity of an α-RgIA4 peptide.

In another example, the binding affinity for the α9α10 nicotinic acetylcholine receptor can be: at least 5% of the binding affinity of the α-RgIA4 peptide, or at least 7.5% of the binding affinity of the α-RgIA4 peptide, or at least 15% of the binding affinity of the α-RgIA4 peptide, or at least 25% of the binding affinity of the α-RgIA4 peptide, or at least 40% of the binding affinity of the α-RgIA4 peptide, or at least 50% of the binding affinity of the α-RgIA4 peptide, or at least 80% of the binding affinity of the α-RgIA4 peptide, or substantially equal to the binding affinity of the α-RgIA4 peptide, or greater than the binding affinity of an α-RgIA4 peptide.

In another example, the protected inter-cysteine sulfur linkage can provide an increase in potency compared to a potency of an α-RgIA4 peptide.

In another example, the analog can provide an α9α10 nicotinic acetylcholine receptor IC₅₀ value that is: substantially equal to an α9α10 nicotinic acetylcholine receptor IC₅₀ value of the α-RgIA4 peptide, or no greater than 2.0× the α9α10 nicotinic acetylcholine receptor IC₅₀ value of the α-RgIA4 peptide, or no greater than 3.0× the α9α10 nicotinic acetylcholine receptor IC₅₀ value of the α-RgIA4 peptide, or no greater than 5.0× the α9α10 nicotinic acetylcholine receptor IC₅₀ value of the α-RgIA4 peptide, or no greater than 15.0× the α9α10 nicotinic acetylcholine receptor IC₅₀ value of the α-RgIA4 peptide, or no greater than 25.0× the α9α10 nicotinic acetylcholine receptor IC₅₀ value of the α-RgIA4 peptide.

In another example, the protected inter-cysteine sulfur linkage can reduce one or more of disulfide bridge scrambling, disulfide bridge degradation, or a combination thereof as compared to an α-RgIA4 peptide, or α-RgIA4 peptide analog without a protected inter-cysteine sulfur linkage.

In another example, the side chain bonding configuration can comprise one or more of a methylene thioacetal, an N-terminal amino acid side chain that is cyclized to a C-terminal amino acid side chain with a lactam bridge, or a combination thereof.

In another example, the side chain bonding configuration can be a methylene thioacetal comprising an inter-cysteine linkage between C^{II} and C^{IV} In another example, the side chain bonding configuration can be an N-terminal amino acid side chain that is cyclized to a C-terminal amino acid side chain with a lactam bridge.

In another example, the N-terminal amino acid can be selected from the group consisting of glutamic acid and aspartic acid.

In another example, the C-terminal amino acid can be selected from the group consisting of lysine and L-2,3-diaminopropionic acid.

In another example, the N-terminal amino acid can be glutamic acid and the C-terminal amino acid can be lysine.

In another example, the protected inter-cysteine sulfur linkage can provide a stability for the α-RgIA4 peptide analog in human serum that is greater than the stability of an α-RgIA4 peptide in human serum, wherein the stability in the human serum is measured by the amount remaining after incubation of 0.1 mg/mL of the α-RgIA4 peptide analog or the α-RgIA4 peptide in 90% human serum AB type and incubated at 37° C. for at least one of 1, 2, 4, 8, 24, 48, or 72 hours.

In another example, the stability in human serum of the α-RgIA4 peptide analog can be at least one or more of 10%, 20%, 40%, 60%, 80%, 100%, 200%, 300%, 400%, 500%, or 1000% greater than the stability of the α-RgIA4 peptide in human serum.

In another example, the protected inter-cysteine sulfur linkage can provide a stability for the α-RgIA4 peptide analog in reduced glutathione that is greater than the stability of an α-RgIA4 peptide in reduced glutathione, wherein the stability in the reduced glutathione is measured by the amount remaining after incubation of 0.1 mg/mL of the α-RgIA4 peptide analog or the α-RgIA4 peptide in 10 equivalents of reduced glutathione in phosphate buffered saline (PBS) having a pH of 7.4 and incubated at 37° C. for at least one of 1, 2, 4, 8, 24, 48, or 72 hours.

In another example, the stability in the reduced glutathione of the α-RgIA4 peptide analog can be at least one or more of 10%, 20%, 40%, 60%, 80%, 100%, 200%, 300%, 400%, 500%, or 1000% greater than the stability of the α-RgIA4 peptide in the reduced glutathione.

In another example, the protected inter-cysteine sulfur linkage can provide an α9α10 nicotinic acetylcholine receptor selectivity that is substantially equal to the α9α10 nicotinic acetylcholine receptor selectivity of an α-RgIA4 peptide.

In another example, the protected inter-cysteine sulfur linkage can provide a α9α10 nicotinic acetylcholine receptor selectivity that is at least one or more of 5×, 10×, 20×, 50×, 100×, or 200× more selective for the α9α10 nicotinic acetylcholine receptor compared to a selectivity of a different nicotinic acetylcholine receptor (nAChR) subtype.

In another example, the different nAChR subtype can be selected from the group consisting of α1β1δε, α2β2, α2β4, α3β2, α3β4 α4β2, α4β4, α6/α3β2β3 and α6/α3β4.

In another example, the protected inter-cysteine sulfur linkage can provide a safety profile that is substantially equal to or greater than the safety profile of an α-RgIA4 peptide, wherein the safety profile is measured by one or more of the analog present in a concentration of 100 μM inhibits less than 25% of the human ether-a-go-go-related gene (hERG) K⁺ channel as measured from an automated-whole cell patch-clamp assay, the analog present in a concentration of 100 μM has inhibitory activity of less than about 20% as measured by a monoamine oxidase (MAO) assay, or the analog present in a concentration of 10 μM has inhibitory activity of less than 20% as measured in a CYP assay.

In another example, the protected inter-cysteine linkage can be one or more of an inter-cysteine linkage between C^{I} and C^{III}, C^{II} and C^{IV}, or a combination thereof.

In another example, the structure can be globular.

In another example, an α-RgIA4 peptide analog can comprise: a recognition finger region comprising D P R; and cystine residues comprising C^{I}, C^{II}, C^{III}, and C^{IV}, wherein: C^{I} and C^{III} are linked by a first inter-cysteine sulfur linkage, and C^{II} and C^{IV} are linked by a second inter-cysteine sulfur linkage; and wherein at least the second inter-cysteine sulfur linkage is protected by a side chain bonding configuration.

In another example, the second inter-cysteine sulfur linkage can comprise methylene thioacetal, an N-terminal amino acid side chain can be cyclized to a C-terminal amino acid side chain with a lactam bridge, or a combination thereof.

In another example, the second inter-cysteine sulfur linkage can comprise methylene thioacetal.

In another example, the analog can comprise the amino acid sequence Xaa₁ C C Xaa₂ D P R C Xaa₃ Xaa₄ Xaa₅ C Xaa₆ (SEQ ID NO: 13), wherein Xaa₁₋₆ is any amino acid other than C.

In another example, the analog can comprise the amino acid sequence Xaa₁ C C Xaa₂ D P R C Xaa₃ Xaa₄ Xaa₅ C Xaa₆ (SEQ ID NO: 14), wherein: Xaa₁ is any proteinogenic or non-proteinogenic amino acid other than C, Xaa₂ is any proteinogenic or non-proteinogenic amino acid other than C, Xaa₃ is a member selected from the group consisting of: (Cit) or any proteinogenic or non-proteinogenic positive amino acid, Xaa₄ is any proteinogenic or non-proteinogenic aromatic amino acid, Xaa₅ is any proteinogenic or non-proteinogenic positive amino acid, and Xaa₆ is any proteinogenic or non-proteinogenic aromatic amino acid.

In another example, the analog can comprise the amino acid sequence Xaa₁ C C Xaa₂ D P R C Xaa₃ Xaa₄ Xaa₅ C Xaa₆ Xaa₇ (SEQ ID NO: 20), wherein Xaa₁₋₇ is any amino acid other than C.

In another example, the analog can comprise the amino acid sequence Xaa₁ C C Xaa₂ D P R C Xaa₃ Xaa₄ Xaa₅ C Xaa₆ Xaa₇ (SEQ ID NO: 21), wherein: Xaa₁ is any proteinogenic or non-proteinogenic amino acid other than C, Xaa₂ is any proteinogenic or non-proteinogenic amino acid other than C, Xaa₃ is a member selected from the group consisting of: (Cit) or any proteinogenic or non-proteinogenic positive amino acid, Xaa₄ is any proteinogenic or non-proteinogenic aromatic amino acid, Xaa₅ is any proteinogenic or non-proteinogenic positive amino acid, Xaa₆ is any proteinogenic or non-proteinogenic aromatic amino acid, and Xaa₇ is any proteinogenic or non-proteinogenic amino acid other than C.

In another example, the analog can comprise the amino acid sequence G C C T D P R C Xaa₃ Xaa₄ Q C Xaa₆ (SEQ ID NO: 15), wherein Xaa₁ is G, Xaa₂ is T, Xaa₅ is Q, and Xaa_{3, 4, or 6} is any amino acid other than C.

In another example, the analog can comprise the amino acid sequence G C C T D P R C Xaa₃ Xaa₄ Q C Xaa₆ (SEQ ID NO: 16), wherein: Xaa₃ is a member selected from the group consisting of (Cit) and R, Xaa₄ is a member selected from the group consisting of (iY) and Y, and Xaa₆ is a member selected from the group consisting of (bhY), Y, and bA.

In another example, the analog can comprise the amino acid sequence G C C T D P R C (Cit) (iY) Q C Y (SEQ ID NO: 18), wherein: Xaa₃ is (Cit), Xaa₄ is (iY), and Xaa₆ is Y.

In another example, the analog can comprise the amino acid sequence G C C T D P R C Xaa₃ Xaa₄ Q C Xaa₆ Xaa₇ (SEQ ID NO: 22), wherein Xaa₁ is G, Xaa₂ is T, Xaa₅ is Q, and Xaa_{3, 4, 6, or 7} is any amino acid other than C.

In another example, the analog can comprise the amino acid sequence G C C T D P R C Xaa₃ Xaa₄ Q C Xaa₆ Xaa₇ (SEQ ID NO: 23), wherein: Xaa₃ is a member selected from the group consisting of (Cit) and R, Xaa₄ is a member selected from the group consisting of (iY) and Y, Xaa₆ is a member selected from the group consisting of (bhY), Y, and bA, and Xaa₇ is R.

In another example, the analog can comprise the amino acid sequence G C C T D P R C R (iY) Q C (bhY) R (SEQ ID NO: 24), wherein: Xaa₃ is R, Xaa₄ is (iY), and Xaa₆ is (bhY).

In another example, the analog can comprise the amino acid sequence G C C T D P R C R (iY) Q C (bA) R (SEQ ID NO: 25), wherein: Xaa₃ is R, Xaa₄ is (iY), and Xaa₆ is (bA).

In another example, an N-terminal amino acid side chain can be cyclized to a C-terminal amino acid side chain with a lactam bridge.

In another example, the N-terminal amino acid can be selected from the group consisting of glutamic acid and aspartic acid.

In another example, the C-terminal amino acid can be selected from the group consisting of lysine and L-2,3-diaminopropionic acid.

In another example, the N-terminal amino acid can be glutamic acid and the C-terminal amino acid can be lysine.

In another example, the analog can comprise the amino acid sequence Xaa₈ Xaa₉ C C Xaa₁₀ D P R C Xaa₁₁ Xaa₁₂ Xaa₁₃ C Xaa₁₄ Xaa₁₅ (SEQ ID NO: 3), wherein Xaa₈₋₁₅ is any amino acid other than C.

In another example, the analog can comprise the amino acid sequence Xaa₈ Xaa₉ C C Xaa₁₀ D P R C Xaa₁₁ Xaa₁₂ Xaa₁₃ C Xaa₁₄ Xaa₁₅ (SEQ ID NO: 4), wherein: Xaa₈ is a member selected from the group consisting of E and D, Xaa₁₅ is a member selected from the group consisting of K and (Dap), and Xaa₉₋₁₄ is any amino acid other than C.

In another example, the analog can comprise the amino acid sequence Xaa₈ Xaa₉ C C T D P R C Xaa₁₁ Xaa₁₂ Q C Y Xaa₁₅ (SEQ ID NO: 5), wherein: Xaa₈ is a member selected from the group consisting of E and D, Xaa₁₀ is T, Xaa₁₃ is Q, Xaa₁₄ is Y, Xaa₁₅ is a member selected from the group consisting of K and (Dap), and Xaa_{9, 11, or 12} is any amino acid other than C.

In another example, the analog can comprise the amino acid sequence Xaa₈ Xaa₉ C C T D P R C Xaa₁₁ Xaa₁₂ Q C Y Xaa₁₅ (SEQ ID NO: 6), wherein: Xaa₈ is a member selected from the group consisting of E and D, Xaa₉ is G or (bA), Xaa₁₁ is R or (Cit), Xaa₁₂ is Y or (iY), and Xaa₁₅ is a member selected from the group consisting of K and (Dap).

In another example, the analog can comprise the amino acid sequence E G C C T D P R C (Cit) Y Q C Y K (SEQ ID NO: 9), wherein: Xaa₈ is E, Xaa₉ is G, Xaa₁₁ is (Cit), Xaa₁₂ is Y, and Xaa₁₅ is K.

In another example, the analog can comprise the amino acid sequence E (bA) C C T D P R C (Cit) Y Q C Y K (SEQ ID NO: 10), wherein: Xaa₈ is E, Xaa₉ is (bA), Xaa₁₁ is (Cit), Xaa₁₂ is Y, and Xaa₁₅ is K.

In another example, the analog can comprise the amino acid sequence E G C C T D P R C (Cit) (iY) Q C Y K (SEQ ID NO: 11), wherein: Xaa₈ is E, Xaa₉ is G, Xaa₁₁ is (Cit), Xaa₁₂ is (iY), and Xaa₁₅ is K.

In another example, the analog can comprise the amino acid sequence E G C C T D P R C R (iY) Q C Y K (SEQ ID NO: 12), wherein: Xaa₈ is E, Xaa₉ is G, Xaa₁₁ is R, Xaa₁₂ is (iY), and Xaa₁₅ is K.

In another example, a composition can comprise a combination of a therapeutically effective amount of an analog as recited with a pharmaceutically acceptable carrier.

In another example, the composition can be suitable for topical, transdermal, intravenous, or subcutaneous administration.

In another example, the composition can further comprise an additional active agent.

In another example, the additional active agent can be a member selected from the group consisting of: an anti-inflammatory agent, an anesthetic, a secondary analgesic peptide, a non-peptide analgesic, and combinations thereof.

In another example, the additional active agent can be present at a concentration of from about 0.0001 wt % to about 10 wt %.

In another example, the composition can be formulated as one of: a solution, a suspension, an emulsion, a gel, a hydrogel, a thermo-responsive gel, a cream, an ointment, a paste, an adhesive, a liquid reservoir, a patch, or a combination thereof.

In another example, the composition can be suitable for subcutaneous injection.

In another example, the pharmaceutically acceptable carrier can include one or more of water, a tonicity agent, a buffering agent, a preservative, or a combination thereof.

In another example, a method of maintaining an α-RgIA4 potency for an α9α10 nicotinic acetylcholine receptor in an α-RgIA4 analog can comprise: protecting inter-cysteine sulfur linkages with a side chain bonding configuration that maintains a recognition finger region of the analog in an α-RgIA4 configuration.

In another example, the analog can bind to the α9α10 nicotinic acetylcholine receptor with an affinity that is: at least 5% of a binding affinity of an α-RgIA4 peptide, or at least 7.5% of the binding affinity of the α-RgIA4 peptide, or at least 15% of the binding affinity of the α-RgIA4 peptide, or at least 25% of the binding affinity of the α-RgIA4 peptide, or at least 40% of the binding affinity of the α-RgIA4 peptide, or at least 50% of the binding affinity of the α-RgIA4 peptide, or at least 80% of the binding affinity of the α-RgIA4 peptide, or substantially equal to the binding affinity of the α-RgIA4 peptide, or greater than the binding affinity of the α-RgIA4 peptide.

In another example, the analog can inhibit the α9α10 nicotinic acetylcholine receptor with an IC₅₀ value that is: substantially equal to an α9α10 nicotinic acetylcholine receptor IC₅₀ value of the α-RgIA4 peptide, or no greater than 2.0× the α9α10 nicotinic acetylcholine receptor IC₅₀ value of the α-RgIA4 peptide, or no greater than 3.0× the α9α10 nicotinic acetylcholine receptor IC₅₀ value of the α-RgIA4 peptide, or no greater than 5.0× the α9α10 nicotinic acetylcholine receptor IC₅₀ value of the α-RgIA4 peptide, or no greater than 15.0× the α9α10 nicotinic acetylcholine receptor IC₅₀ value of the α-RgIA4 peptide, or no greater than 25.0× the α9α10 nicotinic acetylcholine receptor IC₅₀ value of the α-RgIA4 peptide.

In another example, protecting the inter-cysteine sulfur linkage can provide a α9α10 nicotinic acetylcholine receptor (nAChR) selectivity that is at least one or more of 5×, 10×, 20×, 50×, 100×, or 200× more selective for the α9α10 nicotinic acetylcholine receptor compared to a selectivity of a different nAChR subtype.

In another example, protecting the inter-cysteine sulfur linkage can provide a stability in human serum of the α-RgIA4 peptide analog of at least one or more of 10%, 20%, 40%, 60%, 80%, 100%, 200%, 300%, 400%, 500%, or 1000% greater than the stability in human serum of an α-RgIA4 peptide.

In another example, protecting the inter-cysteine linkage can comprise protecting one or more of an inter-cysteine linkage between C^{I} and C^{III}, C^{II} and C^{IV}, or a combination thereof.

In another example, protecting the inter-cysteine sulfur linkages can comprise inserting a methylene thioacetal between C^{II} and C^{IV}.

In another example, protecting the inter-cysteine sulfur linkages can comprise creating a lactam bridge between an N-terminal amino acid and a C-terminal amino acid.

In another example, a method for treating in a subject, a condition that is responsive to α9α10 nicotinic acetylcholine receptor binding, can comprise administering a therapeutically effective amount of the composition as recited to the subject.

In another example, the condition can be pain.

In another example, the pain can be neuropathic pain including one or more of: chemo-induced neuropathy (CIPN), diabetic neuropathy, arthritic neuropathy, osteoarthritic neuropathy, or a combination thereof.

In another example, the pain can be HIV pain.

In another example, the pain can be pain associated with leprosy.

In another example, the pain can be one or more of post-surgical pain, or post-traumatic pain.

In another example, the condition can be spinal polyradiculopathy.

In another example, the condition can be postherpetic neuralgia.

In another example, the condition can be trigeminal neuralgia.

In another example, the condition can be complex regional pain syndrome.

In another example, the condition can be cancer.

In another example, the cancer can include one or more of: epithelial cancer, lung cancer, breast cancer, or a combination thereof.

In another example, the condition can be multiple sclerosis.

In another example, the condition can be inflammation.

In another example, the inflammation can be mediated by immune cells, associated with rheumatism, or a combination thereof.

In another example, the treatment can provide a reduction in symptoms of at least 10% within a selected amount of time after administration.

In another example, the therapeutically effective amount of the composition can be administered to the subject 1 to 5 times per day.

In another example, the therapeutically effective amount of the composition can be administered to the subject according to a dosage regimen of at least once per day for a duration of from about a single day to about 3 months.

In another example, the therapeutically effective amount of the composition can be administered as a subcutaneous dosage form, a transdermal dosage form, a topical dosage form, an intravenous dosage form, or a combination thereof.

In another example, a composition for use in the treatment of a condition in a subject that is responsive to α9α10 nicotinic acetylcholine receptor binding, can comprise a therapeutically effective amount of the composition as recited to the subject.

In another example, use of a composition in the manufacture of a medicament for the treatment of a condition in a subject that is responsive to α9α10 nicotinic acetylcholine receptor binding can comprise: a therapeutically effective amount of the composition as recited to the subject.

### Experimental Examples

The following examples are provided to promote a more clear understanding of certain embodiments of the present disclosure, and are in no way meant as a limitation thereon.

### Example 1-A: Design and Synthesis of Conformationally Constrained α-RgIA Analogues

### Methods:

Peptide Synthesis. Peptides were synthesized using automated Fmoc SPPS chemistry on synthesizer (Syro I). The first amino acid was coupled manually to onto 2-CTC resin (substitution=0.77 mmol/g) and the resin was capped with MeOH to a final substitution of 0.4 mmol/g. Briefly, 250 mg of 2-CTC resin (substitution=0.77 mmol/g) was swelled and washed in DCM for 30 min. The resin was drained and followed by adding in a solution of specific Fmoc protected amino acid (Fmoc-AA-OH=0.1 mmol, DIEA=0.2 mmol in DCM=4 mL) and incubated at room temperature for 1.5 h. Then the resin was washed with DMF and DCM multiple times and incubated with 5 mL of DCM containing 16% v/v MeOH and 8% v/v DIEA for 5 min. This action was repeated for 5 times before thoroughly washed with DCM and DMF. Then the resin was set to the synthesizer for automated synthesis. Coupling reactions were performed using HATU (5.0 eq.), DIEA (10.0 eq.) and Fmoc-AA-OH (5.0 eq.) in DMF (5 mL per 0.1 mmol amino acid bonded resin) with 15 min heating to 70° C. (50° C. for Cys and Allyl and Aloc protected amino acids). Deprotection reaction was performed using 20% (v/v) Piperidine in DMF (4 mL), 5 min for 2 rounds at room temperature.

Cleavage. Peptides were cleaved off from the resin by treatment with a cocktail buffer that consisted of TFA/H₂O/TIPS/EDT=95:2:2:1 for 2.5 h at room temperature (3.0 mL per 0.1 mmol sequence bonded resin). The obtained peptide-TFA solution was then filtered and precipitated out into cold ethyl ether, centrifuged, and washed with ethyl ether for at least 2 times before it was dried in vacuum. The crude product was then purified by RP-HPLC.

LC/MS analysis. Characterization of peptides was performed by LC/MS on an Xbridge C18 5 μm (50×2.1 mm) column at 0.4 mL/min with a H₂O/ACN gradient in 0.1% FA on an Agilent 6120 Quadrupole LC/MS system. Fractions collected from HPLC runs were also analyzed by LC/MS.

HPLC purification methods and purity check. All samples were analyzed by the following conditions unless otherwise specified: Semi-preparative reverse phase HPLC of crude peptides was performed on Jupiter 5μ C18 300 Å (250×10 mm) column at 3.0 mL/min with a H₂O/ACN gradient containing 0.1% TFA from 5% to 35% of ACN over 45 minutes on an Agilent 1260 HPLC system. The purified fractions containing the targeted product were collected and lyophilized using a Labconco Freeze Dryer. All purity assessments, isomer co-injections, and stability assay analysis were performed by HPLC on a Phenomenex Gemini C18 3 μm (110 Å 150×3 mm) column.

On resin orthogonal deprotection and lactamization. Method 1: The allyl ester (OAll) and allyl carbamate (NHAloc) were removed on resin using Pd(PPh₃)₄ (0.1 eq.) and DMBA (4.0 eq.) in DCM for 2 h and this reaction was repeated for 2 rounds. Method 2: The Pd mediated deprotection is not compatible with 3-iodo-Tyr containing sequence (the de-Iodo product was a major possibly because of Pd insertion and reduction). Thus, O(Dmab) and NH(ivDde) were used as the orthogonal protection pair. The loaded resin was incubated in 5% hydrazine in DMF for 4 h and the action was repeated once. Lactam cyclization was performed on resin under the cyclization condition of PyBOP/HOBt/DIEA (2:2:2.4 eq.) in DMF and >6 h agitation on rotator used for completion. The reaction conversion was monitored by micro-cleavage and checked by LC/MS.

Air-oxidative disulfide bond formation. Peptide with two free Cysteines was oxidized by aerating in a 0.01 M Na₂HPO₄ buffer (pH=8.0) containing 5% DMSO at room temperature for >48 h. The reaction progress was monitored by LC/MS. Upon completion, the reaction mixture was purified by RP-HPLC using the method mentioned above.

I₂-mediated disulfide bond formation. To the stirred Bis-Acm-protected peptide solution in AcOH: H₂O (80%:20% v/v, 1.00 mM) was added 12 (10.0 eq.) dissolved in AcOH in a dropwise manner. The reaction was stirred at room temperature for 10 min and monitored by LC/MS. The excess 12 was quenched by the addition of Ascorbic acid solution (1.0 M) until the mixture became colorless. Then the mixture was diluted with H₂O (equal volume to the reaction mixture) and purified by RP-HPLC.

### Results and Discussion:

Based on the goal to engineer a fully-active, cyclic α-RgIA4 analogue, the NMR structure (PDB 2JUQ) was examined and a recent receptor co-crystalized structure (PDB 6HY7) of α-RgIA. Holding a distance of around 11.6 A, both N- and C-termini of α-RgIA reach out away from the pharmacophore. In order to accommodate the existing backbone geometry, additional amino acids at both ends would be used in order to span this distance so that the ideal linker minimally perturbs the backbone to maintain potency. Therefore, a series of side chain cyclized peptides was synthesized following a designed synthetic route depicted in Scheme 1. The newly introduced lactam bridge was synthesized on resin followed by a two-operation liquid phase oxidation process in which a regioselective disulfide-bond arrangement was applied to afford the globular isomer. In detail, as illustrated in FIG. 2, side-chain protected P1 was synthesized through automated Fmoc solid-phase peptide synthesis (SPPS) on 2-chlorotrityl chloride (2-CTC) resin with N-terminal Fmoc removal and re-protected with Boc. The terminal side chain amine and acid were then orthogonally deprotected (PG₁ and PG₂) and further cyclized to form the lactam bridged molecule complex P2. The lactam cyclized peptide was generated through cleavage, purification, and then underwent air oxidation to afford bicyclic product P3. Finally, fully folded P4 was generated through an in situ iodine oxidative deprotection-disulfide formation.

### Example 1-B: In Vitro and In Vivo Biological Evaluation of Synthesized Peptides

### Methods:

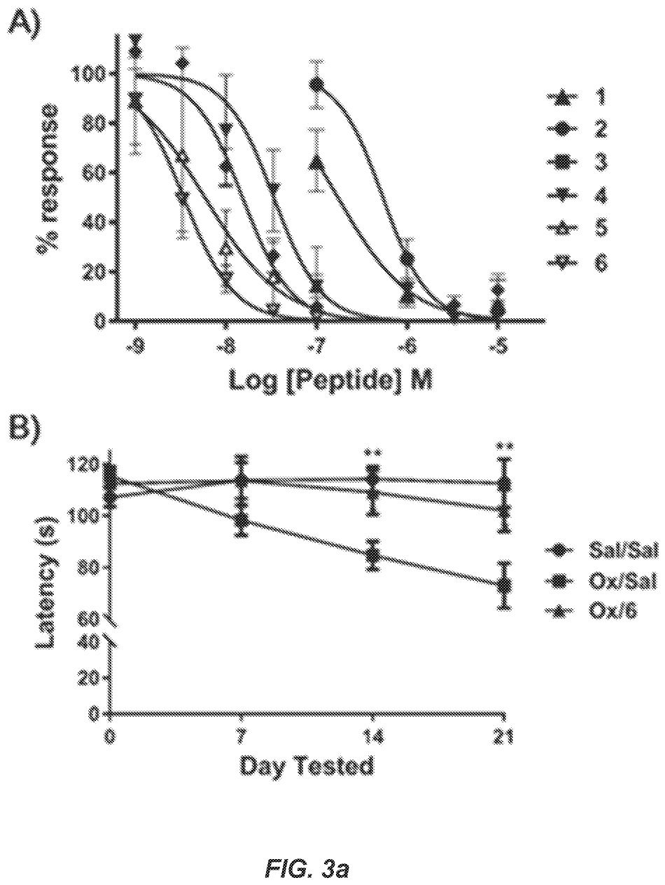

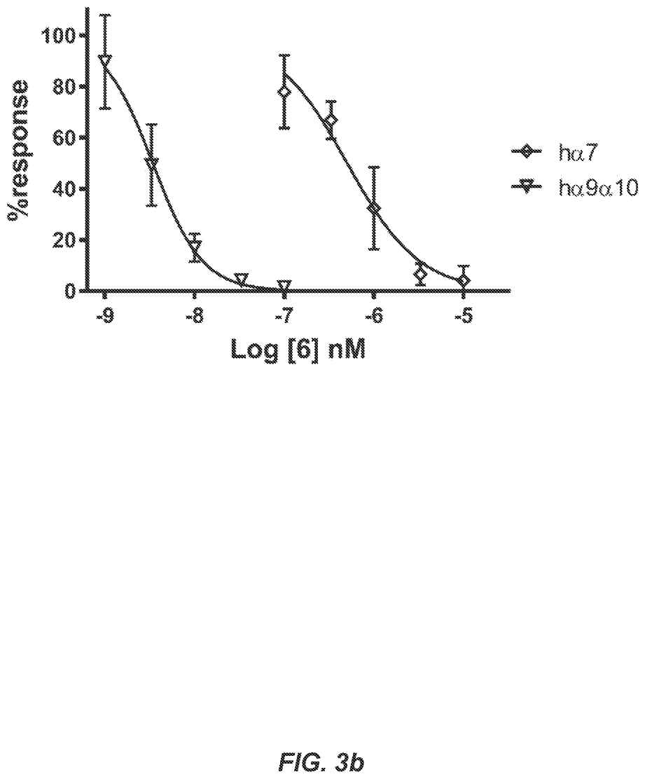

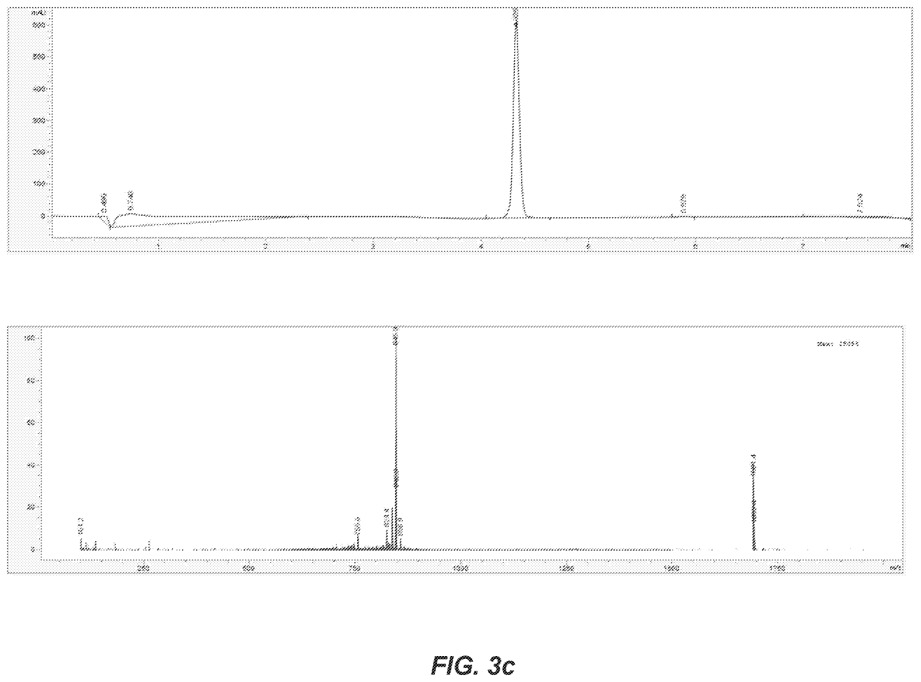

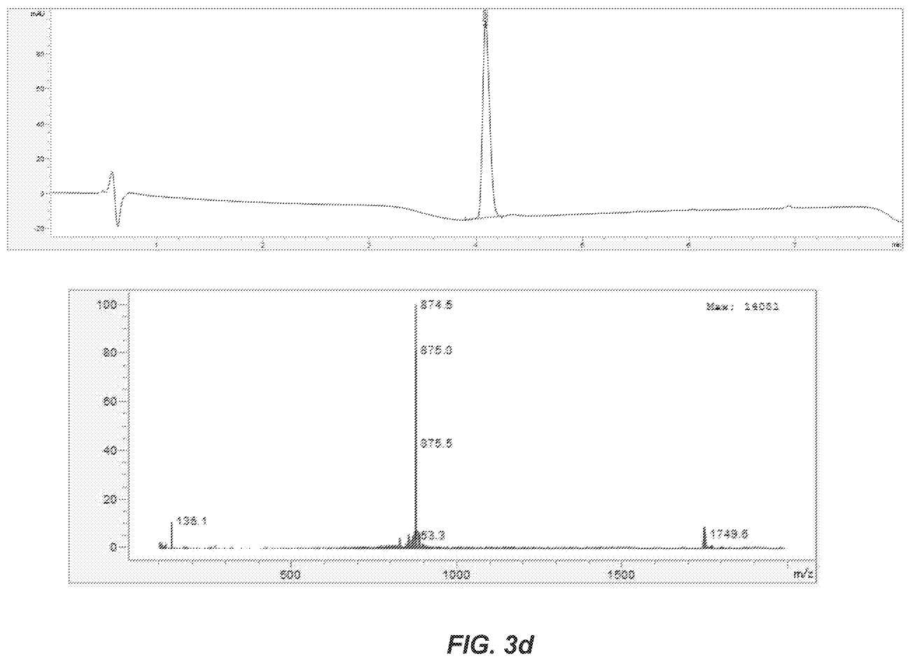

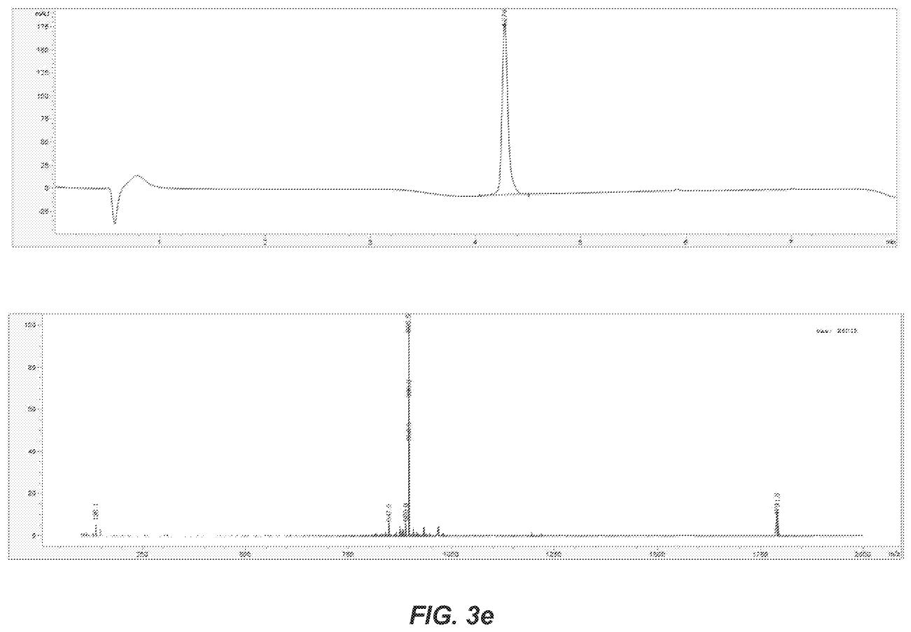

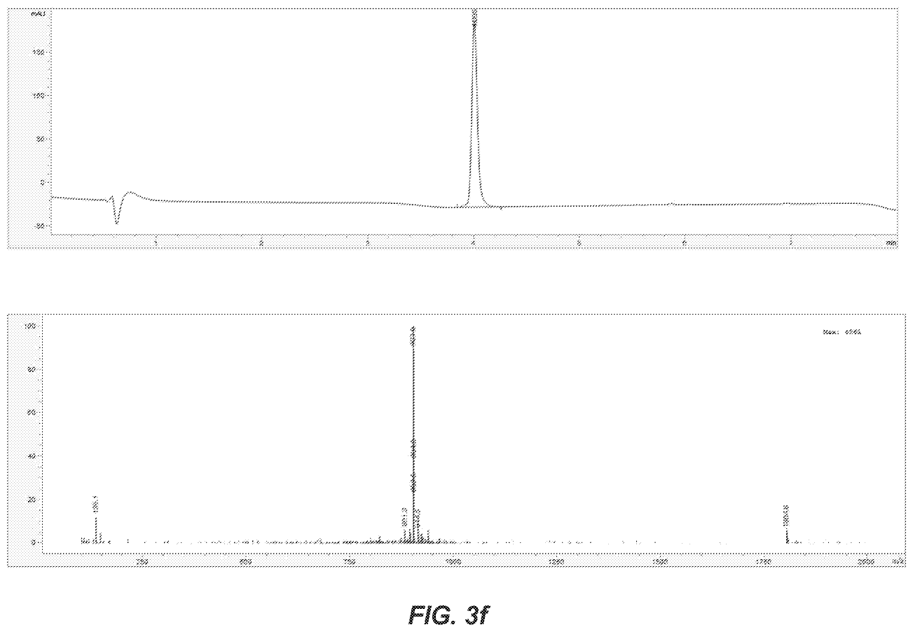

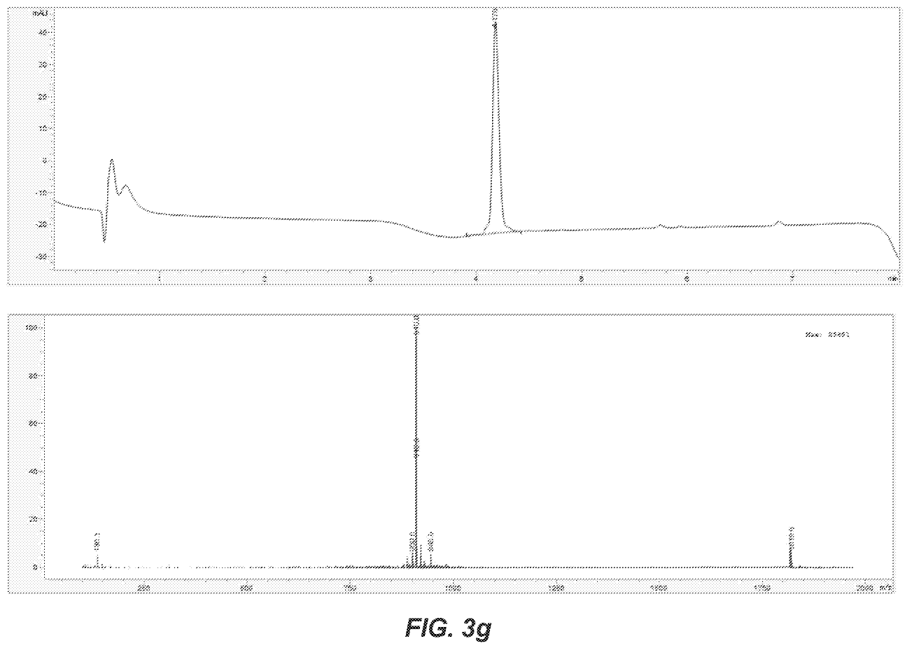

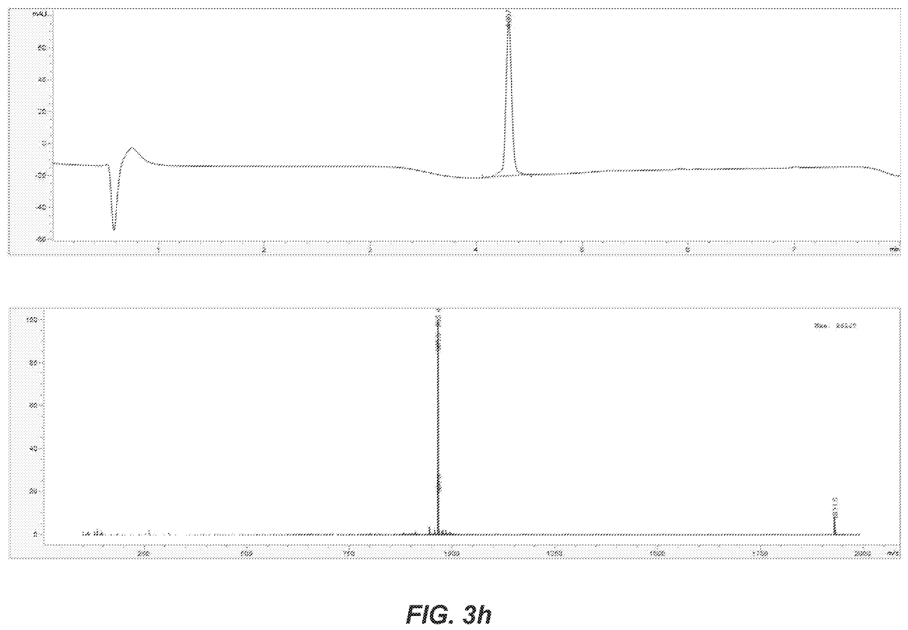

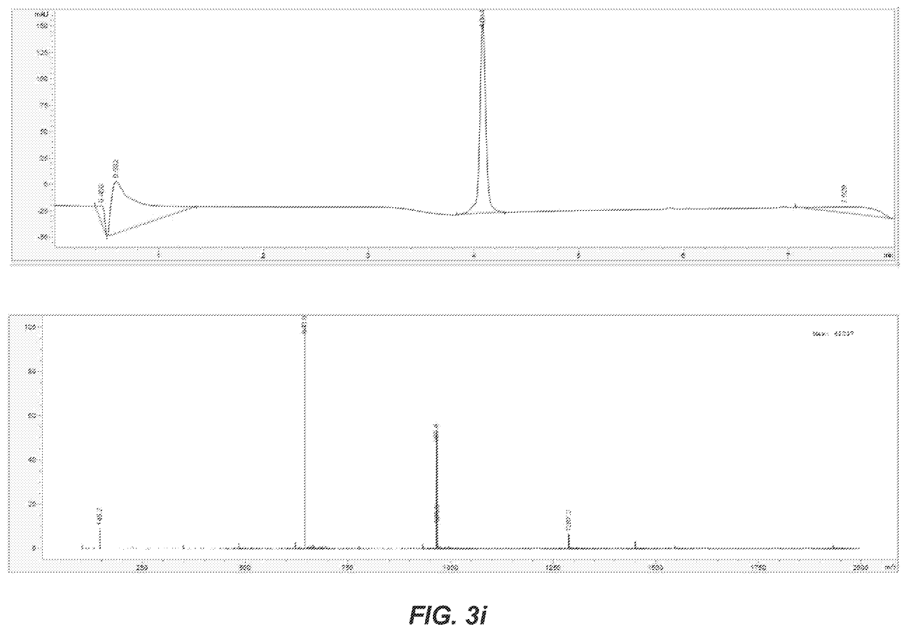

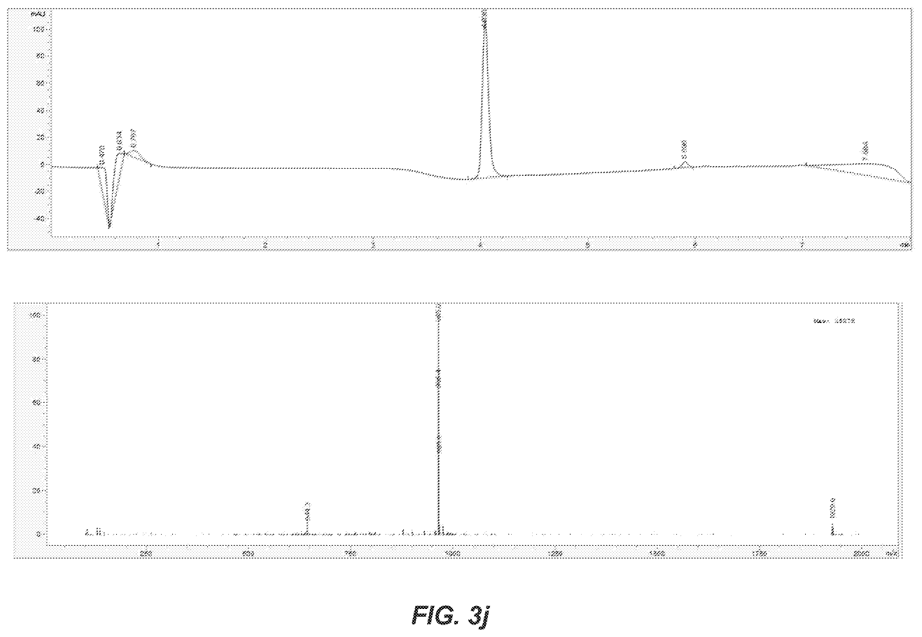

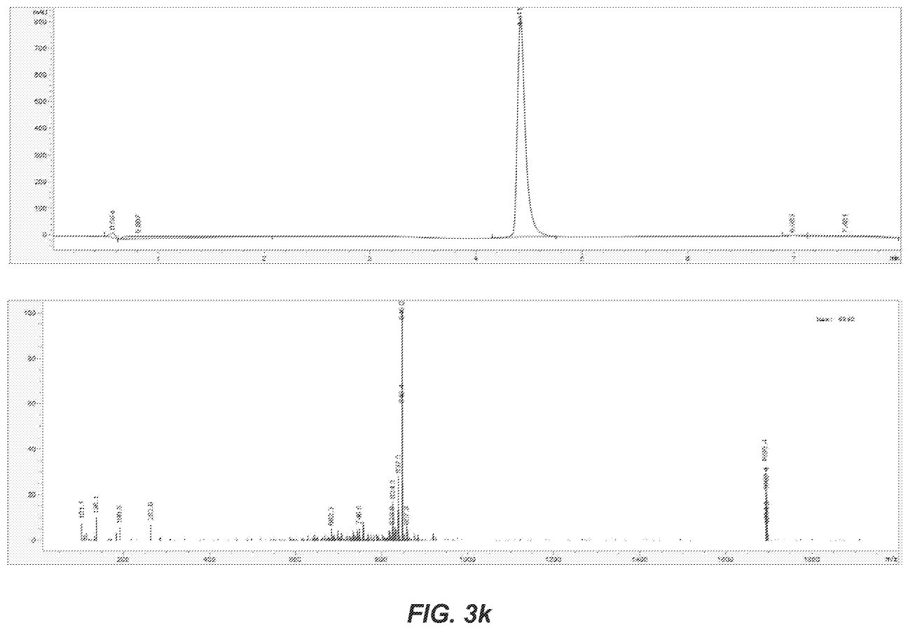

Compound characterization. All analogues synthesized and studied in this research were determined with purity ≥95% by HPLC. Molecular weights were measured by ESI-MS. [M+H]⁺ M/Z (Da): RgIA4, Calc 1691.6, Found 1691.4; Analogue 1: Calc 1749.1, Found 1749.6; Analogue 2, Calc 1791.0, Found 1791.5; Analogue 3, Calc 1804.1, Found 1804.6; Analogue 4, Calc 1819.1, Found 1819.6; Analogue 5, Calc 1931.0, Found 1931.5; Analogue 6, Calc 1929.0, Found 1929.8; RgIA4[1,4], Calc 1691.6, Found 1691.4; Analogue 6[1,4], Calc 1929.0, Found 1929.6. The LC-Chromatogram and MS-Spectrum for each of the purified peptides are shown in FIGS. 3C to 3K as summarized in Table 1B-1.

TABLE 1B-1
Molecular Weight^{a}

| | Calculated | Observed | |
|---|---|---|---|
| Analogue No. | [M + H]⁺ | [M + H]⁺ | Purity^{b} |
| RgIA4 | 1691.6 | 1691.4 | >99% |
| 1 | 1749.1 | 1749.6 | >99% |
| 2 | 1791.0 | 1791.5 | >99% |
| 3 | 1804.1 | 1804.6 | >99% |
| 4 | 1819.1 | 1819.6 | >99% |
| 5 | 1931.0 | 1931.5 | >99% |
| 6 | 1929.0 | 1929.8 | >99% |
| RgIA4[1, 4] | 1691.6 | 1691.4 | >96% |
| 6[1, 4] | 1929.0 | 1929.6 | >95% |

^{a}Determined by ESI mass spectrometry.
^{b}Determined by RP-HPLC.

Oocyte receptor expression. X. laevis oocytes were micro-injected with cRNA encoding the selected nAChR subunits. For all the human heterologous nAChRs oocytes were injected with 15-25 ng equal parts of each subunit, and for homologous human α7 oocytes were injected with 50 ng of α7 encoding cRNA. Oocytes were incubated at 17° C. for 1-3 days in ND96 prior to use.

Electrophysiological Recordings. Injected oocytes were placed in a 30 μL recording chamber and voltage clamped to a membrane potential of −70 mV. ND96 (96.0 mM NaCl, 2.0 mM KCl, 1.8 mM CaCl₂), 1.0 mM MgCl₂, 5 mM HEPES, pH 7.5) with 0.1 mg/mL BSA was gravity perfused through the recording chamber at −2 mL/min. A one second pulse of ACh was applied to measure receptor response, with pulses occurring every minute. ACh was applied at a concentration of 100 μM for all subtypes, with the exception of 200 μM for α7 and 10 μM for the muscle subtype. A baseline ACh response was established, and then the ND96 control solution was switched to a ND96 solution containing the various concentrations of test peptides. During perfusion of the peptide-containing solutions, ACh pulses continued once per minute to assess for block of the ACh-induced response. ACh responses were measured in the presence of a peptide concentration until the responses reached steady state; an average of three of these responses compared to the baseline response was used to determine percent response. Due to limited material, for 10 μM concentration testing, 3 μl of 100 μM peptide was introduced into the 30 μl recording chamber with the ND96 flow stopped. After 5 minutes of incubation, the ND96 flow and ACh pulses resumed to measure any block by peptide. All concentration-response analysis was performed with GraphPad Prism software; values, including the resulting IC₅₀ were calculated using a non-linear regression (curve fit) sigmoidal dose-response (variable-slope).

Oxaliplatin-Induced Cold Allodynia. Oxaliplatin was dissolved at 0.875 μg/μl in 0.9% sterile saline. Analogue 6 was dissolved at 0.02 μg/μl in 0.9% sterile saline. CBA/CaJ mice were injected daily (excluding weekends) i.p. with oxaliplatin (3.5 mg/kg) or 0.9% saline (vehicle). Mice were also injected daily s.c. with analogue 6 (80 μg/kg) or 0.9% saline as control. All compounds were blinded for the experimenter in this study. The study began with an initial baseline cold sensitivity testing on a Wednesday, and injections were administered during the first week on Wednesday, Thursday, and Friday. Injections continued Monday-Friday for two more weeks, with testing on Wednesdays 24 hours after the previous day's injection. The final week, injections occurred on Monday and Tuesday, and the final testing day occurred 24 hours later.

Cold Plate Test. Testing was performed using a hot/cold plate machine purchased from IITC Life Science. Test mice were allowed to acclimate to the testing chamber with the plate held at room temp (23° C.) until investigative behavior subsided. Temperature was then lowered on a linear ramp at a rate of 10° C. per minute. The test was stopped when the mouse lifted both forepaws and vigorously shook them or repeatedly licked the footpad. Lifting of one forepaw at a time or alternating back and forth between paws was not scored and the testing continued. Final time and temperature was recorded and the resulting data was plotted using Graphpad Prism. Data was analyzed using a one-way ANOVA with Dunnett's Multiple Comparison Test. P-values were, * P<0.05, ** P<0.01, and *** P<0.001 for significant difference from oxaliplatin/saline control.

### Results and Discussion:

Prior failures in clinical trials of some α-CTx-based drug candidates demonstrated that surmounting the different sensitivities of human versus rodent nAChRs is a significant hurdle and confounding factor in the development of CTx-based analgesics. To address this, the synthesized analogues were tested by two-electrode voltage-clamp electrophysiology of human α9α10 nAChR expressing X. laevis oocytes. The IC₅₀ values generated from each concentration-response curve, as shown in FIG. 3a-A and FIG. 3a-B are listed in Table 1B-2 (with [ ] indicating cyclization and \hat{ } indicting the C terminal).

TABLE 1B-2

| Pep- | | IC₅₀ | 95% CI |
|---|---|---|---|
| tide | Sequences | (nM) | (nM) |
| RgIA4 | GCCTDPRC(Cit) | 1.5 | 0.5-2.5 |
| | (iY)QCY\hat{ } | | |
| 1 | [DGCCTDPRC(Cit) | 171.9 | 98.4-300.4 |
| | YQCY(Dap)]\hat{ } | | |
| 2 | [DGCCTDPRC | 539.3 | 347.9-836.2 |
| | (Cit)YQCYK]\hat{ } | | |
| 3 | [EGCCTDPRC | 15.2 | 11.4-20.4 |
| | (Cit)YQCYK]\hat{ } | | |
| 4 | [E(^{b}A)CCTDPR | 33.7 | 20.9-54.3 |
| | C(Cit)YQCYK]\hat{ } | | |
| 5 | [EGCCTDPRC | 5.9 | 3.4-10.0 |
| | (Cit)(iY)QCYK]\hat{ } | | |
| 6 | [EGCCTDPRCR | 3.4 | 2.6-4.4 |
| | (IY)QCYK]\hat{ } | | |

Analogues 1 to 4 were synthesized to identify the optimal linker configuration. The most potent analogue 3, cyclized with terminal [Glu-Lys] side chains, is 10-fold less potent compared with α-RgIA4. The bioactivity drops significantly in analogue 1 [Asp-Dap]and 2 [Asp-Lys] when shorter linkers are generated, which are likely caused by strains and perturbations in the backbone. Analogue 4 with one CH₂ unit lengthened linker by replacing Gly1 with Ala1 at the N-terminal residue of the molecules also resulted in potency drop, although not as drastic as the analogues with shorter linkers. With a good linker identified as [Glu-Lys], analogue 5 was synthesized with 3-iodo-tyrosine mutation which is a residue that can increase potency on human α9α10 nAChR. The potency of analogue 5 reached 5.9 nM and was further reduced to 3.4 nM (analogue 6) when Cit9 was mutated back to Arg9 (in accordance with α-RgIA5). Testing analogue 6 on other human nAChR subtypes to measure its selectivity showed >10 μM potency for all subtypes except for α7 (IC₅₀=504.0 nM, 150-fold reduction) which is also a pain-associated nAChR subtype, as shown in FIG. 3b. This result indicates that the current cyclization strategy produces analogue 6 with both retained potency and good receptor selectivity, as shown in Table 1B-3.

TABLE 1B-3

| nAChR | | | |
|---|---|---|---|
| subtype^{α} | IC₅₀ (nM) | | Fold to α9α10 |
| α2β4 | >10000^{b} | | >1000 |
| α3β4 | >10000^{b} | | >1000 |
| α2β4 | >10000^{b} | | >1000 |
| α4β4 | >10000^{b} | | >1000 |
| α4β4 | >10000^{b} | | >1000 |
| α6/α3β2β3 | >10000^{b} | | >1000 |
| α6/α3β4 | >10000^{b} | | >1000 |
| β4β4 | >10000^{b} | | >1000 |
| Muscle type | >10000^{b} | | >1000 |
| α7 | 504 | (360-707)^{c} | 148 |
| α9α10 | 3.4 | (2.6-4.4)^{c} | — |

^{α}All receptors are human type.
^{b}The inhibition was <50% at 10 μM.
^{c}Numbers in parentheses are 95% confidence intervals.

The in vivo pain-relieving effect of analogue 6 was assessed in the oxaliplatin-induced peripheral neuropathic pain rodent model. As shown in FIG. 3a-B, oxaliplatin administration in mice produced cold allodynia which led to progressively reduced latency on cold plate testing while a daily co-administration of analogue 6 significantly prevented the cold allodynia.

### Example 1-C: Human Serum Stability of RgIA4 and Analogue 6

### Methods:

In Vitro Human Serum Stability Test. Peptides (RgIA4 and analogue 6) were dissolved in H₂O (1.0 mg/mL) and 100 μL of this solution was added into 900 μL of human serum from human male AB plasma which was first-time defrosted, sterile filtered, and pre-centrifuged at 13,000 rpm for 15 min to remove lipid. Final peptide concentration was 0.1 mg/mL. Solutions were then incubated in 37° C. water bath and individual 100 μL of the solution was taken up at certain, pre-determined time points and treated with 300 μL ACN and cooled on ice for 30 mins. The suspension was centrifuged at 13,000 rpm for 5 mins at room temperature. Then 10 μL of supernatant was taken up and dissolved in 10 μL of Buffer A (0.1% TFA in H₂O) to make the HPLC sample. The samples were analyzed by HPLC (injection volume=15 μL; column: Phenomenex, 150 mm×4.6 mm, 100 A, 5 μm) with a linear gradient of 5-50% B over 8 mins (A=H₂O+0.1% FA and B=ACN+0.1% FA; 0.4 mL/min flow rate). Peptide peak areas were integrated on 220 nm and the % of peptide left, compared to the initial was graphed against the time. The serum stability experiments were repeated independently for 3 times of each peptide. Data analysis was performed with GraphPad Prism software.

### Results and Discussion:

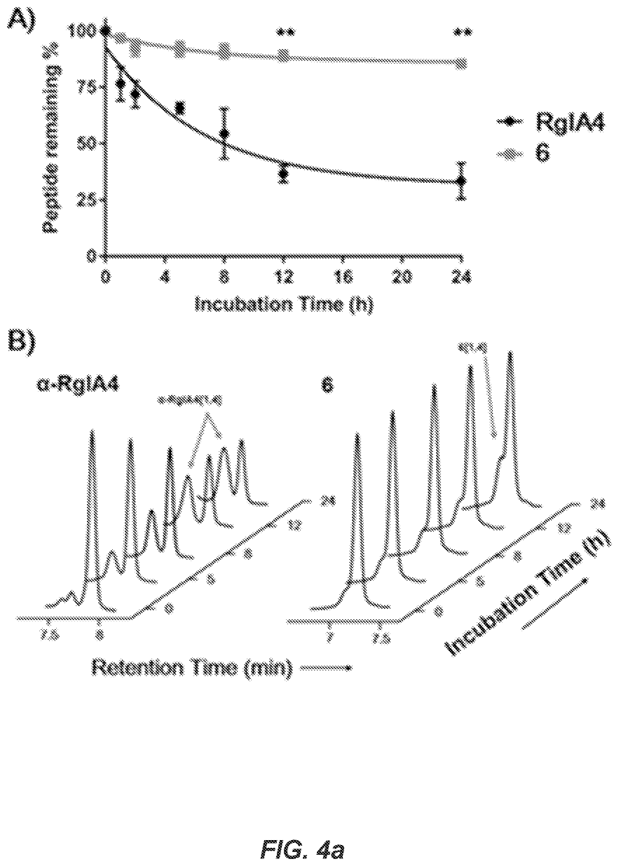

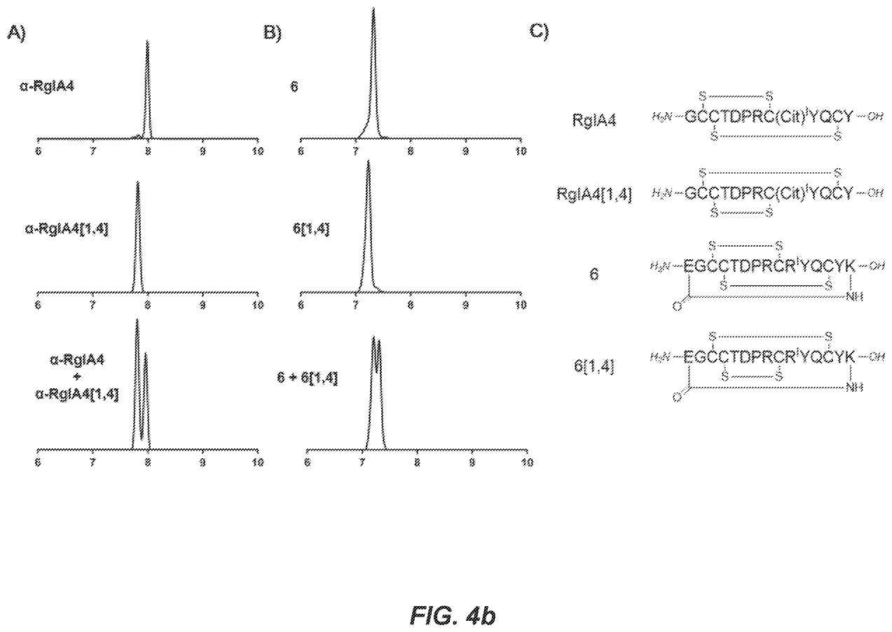

Thiol-induced disulfide scrambling and proteolytic degradation in human plasma are two major threats to disulfide-rich peptide drugs. To determine how the newly introduced conformational constraint influences metabolic stability, we carried out in vitro human serum stability assay on the most potent analogue 6 in comparison with α-RgIA4. As shown in FIG. 4a-A, analogue 6 exhibited a dramatically increased stability over α-RgIA4. Furthermore, a striking disulfide scrambling suppression was observed by HPLC analysis as shown in FIG. 4a-B. The front peaks are scrambled products [1,4] which were identified with isomer co-injection, as shown in FIG. 4b-A, FIG. 4b-B, and FIG. 4b-C. Over half of α-RgIA4 scrambled into its ribbon isomer α-RgIA4[1,4] whereas less than 10% of analogue 6 was scrambled, as shown in FIG. 4a-A. Taken together, this data indicates the side chain cyclization in analogue 6 greatly inhibited both proteolytic degradation and disulfide scrambling.

### Example 1-D: NMR Analysis and Structure Determination

### Methods:

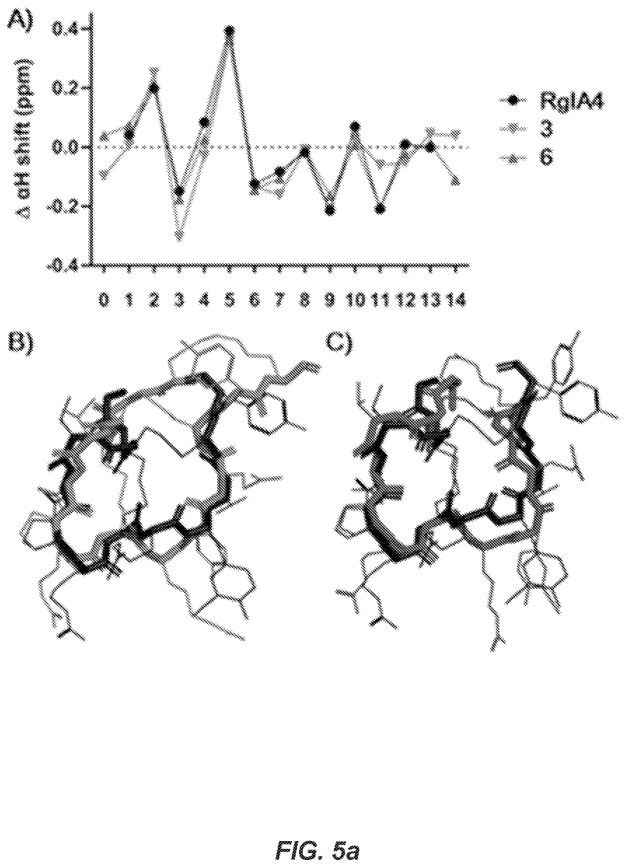

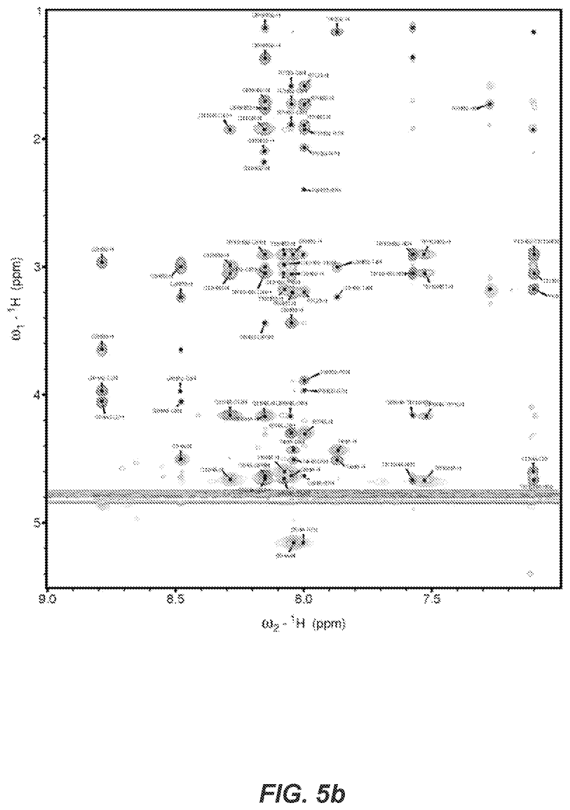

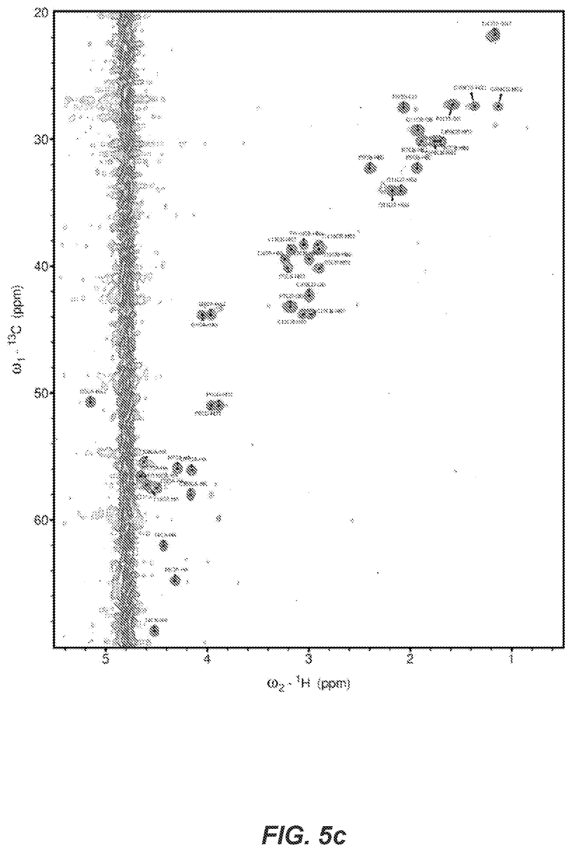

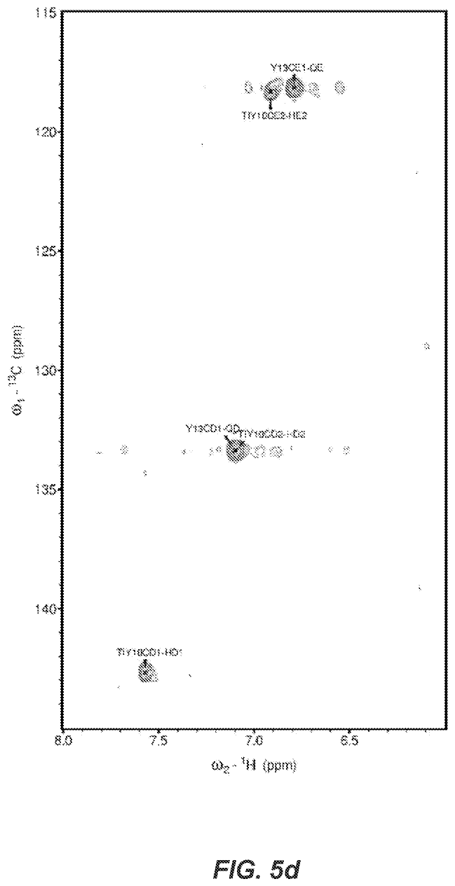

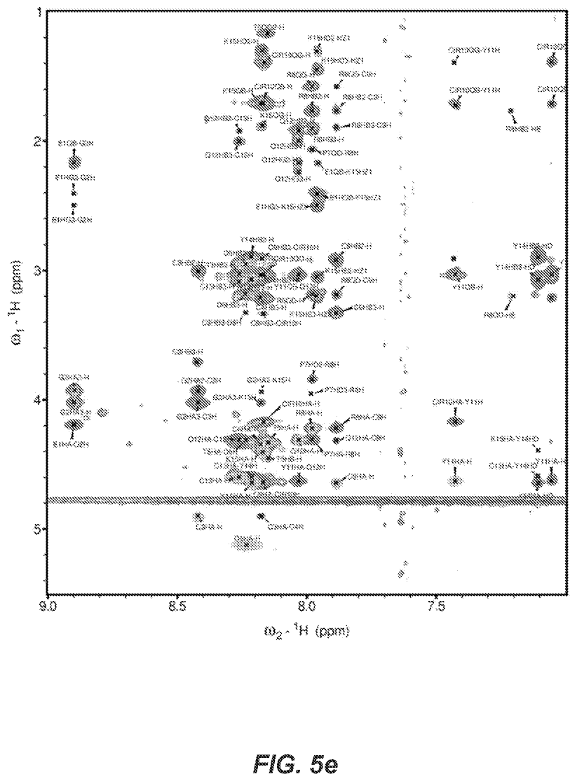

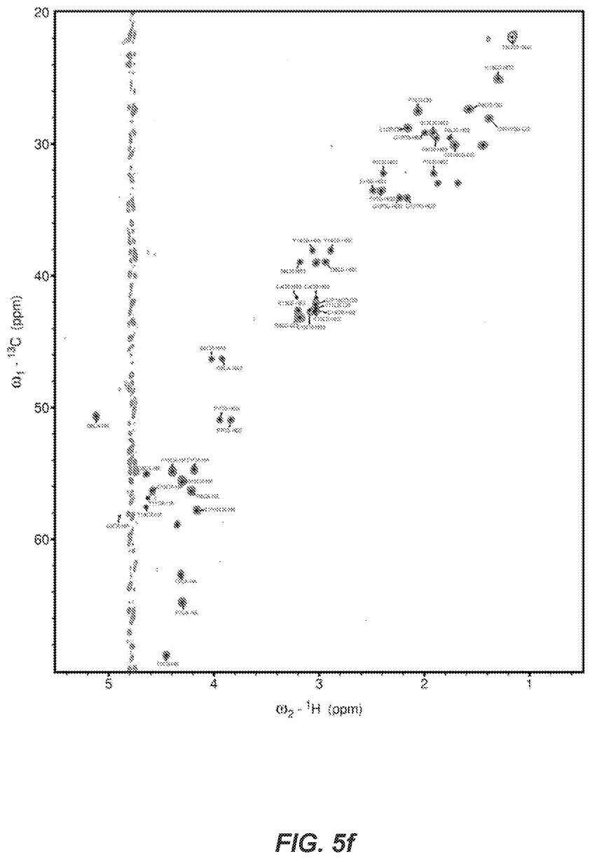

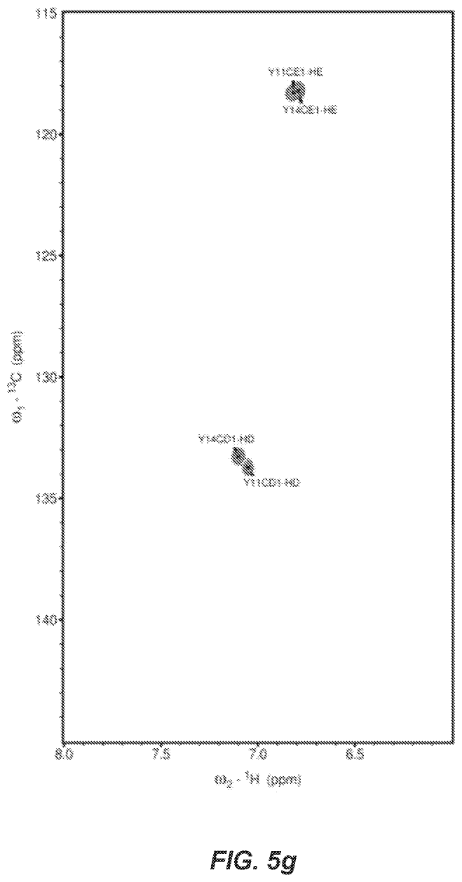

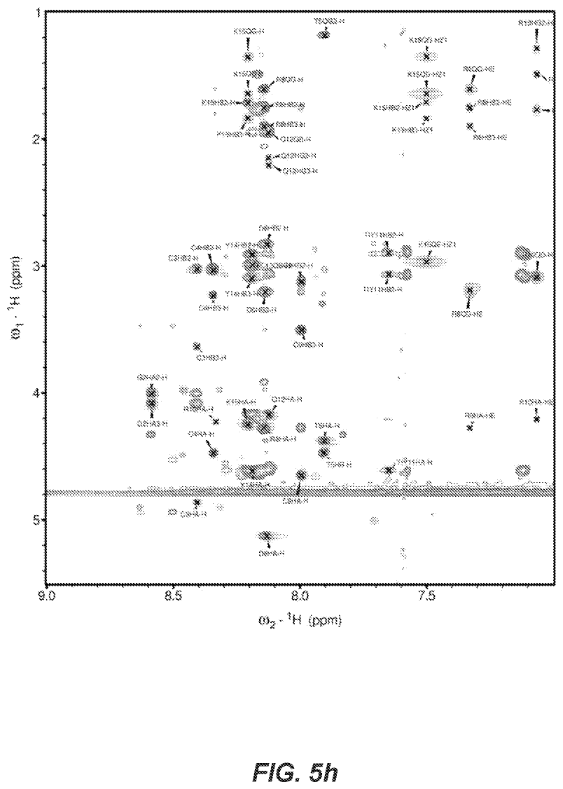

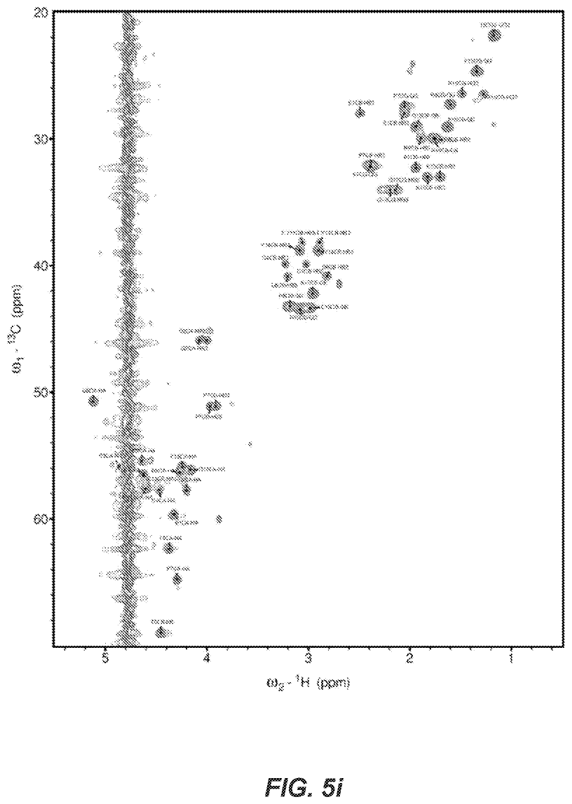

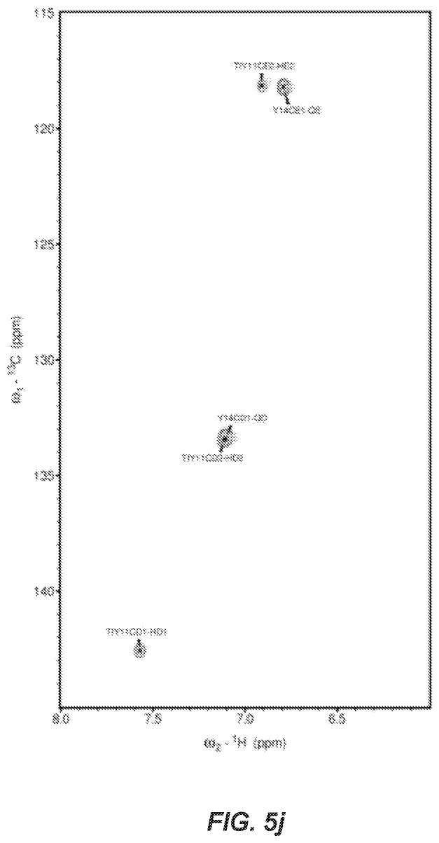

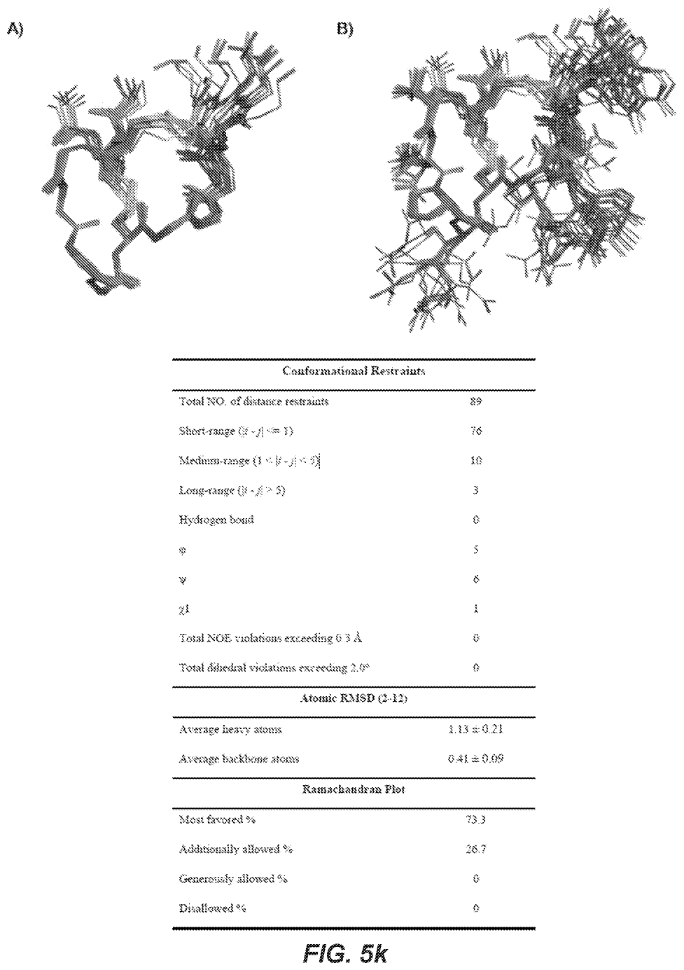

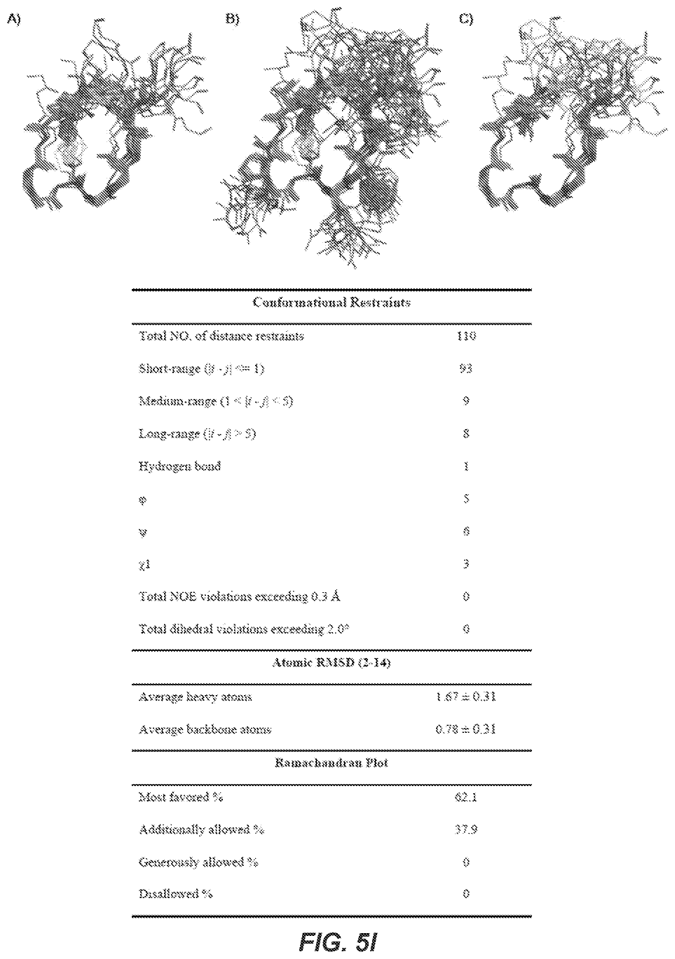

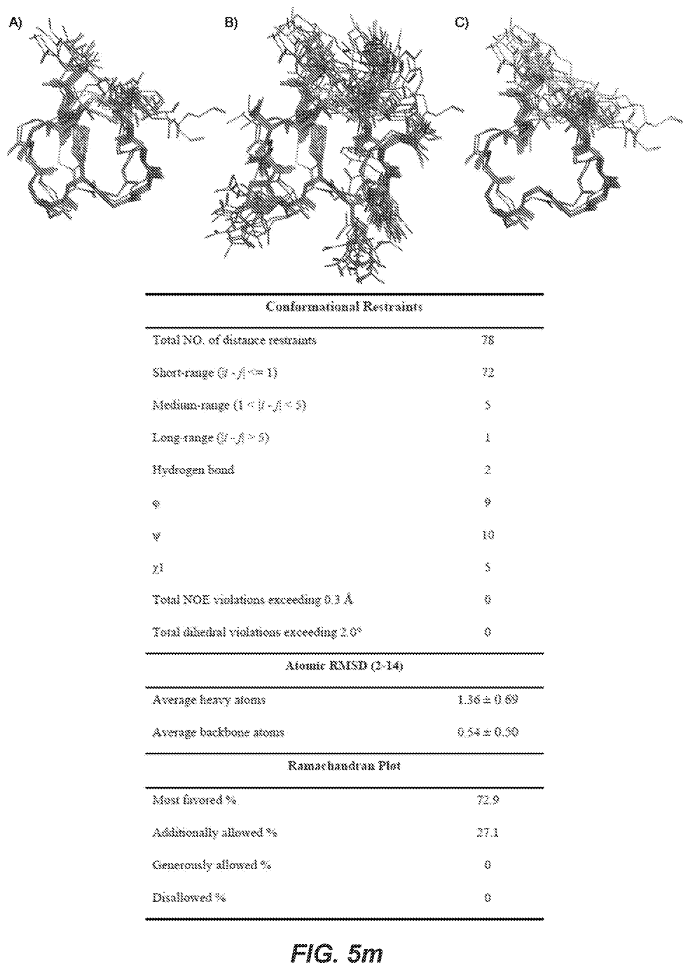

NMR Spectroscopy. Peptide samples were prepared in a pH 3.5 buffer consist of (20 mM Na₂HPO₄, 50 mM NaCl, 50 μM NaN₃ and 0.1 mM EDTA) containing 10% D20 at 2.0 mM (uncorrected for isotope effects). Spectra were recorded on an Inova 500 and 600 MHz spectrometer at 298 K. Spectrometers were set with VnmrJ4.0. The 2D experiments including TOCSY (80 ms), gCOSY, NOESY (200 ms), g11-NOESY, and 13C-HSQC were generated. Samples were loaded in Shigemi tube for data collection and water suppression was achieved using excitation sculpting with gradients. Spectra were processed with software NMRPipe and chemical shifts were assigned with SPARKY, as shown in Table 1D-1 for α-RgIA4, Table 1D-2 for Analogue 3, and Table 1D-3 for Analogue 6. Overlay of the amide regions of TOCSY (blue) and NOESY (red) with HSQC aliphatic region and aromatic region. Assignments were made using SPARKY. See e.g., FIGS. 5b to 5j.

TABLE 1D-1
α-RgIA4

| | C_{α} | C_{β}, | Other C | H_{N} | H_{α}, | H_{β} | Other H |
|---|---|---|---|---|---|---|---|
| G1 | 43.84 | — | — | — | 3.978 4.052 | — | — |
| C2 | ND | ND | — | 8.788 | 4.849 | 2.954 3.845 | — |
| C3 | 57.49 | 39.46 | — | 8.479 | 4.582 | 3.005 3.239 | — |
| T4 | 62.05 | 68.76 | C_{γ} 21.75 | 7.869 | 4.434 | 4.520 | H_{γ} 1.165 |
| D5 | 50.73 | 40.16 | — | 8.040 | 5.154 | 2.906 3.201 | — |
| P6 | 64.80 | 32.30 | C_{γ} 27.51; C [symbol] 50.99 | — | 4.316 | 1.932 2.401 | H_{γ} 2.070; H [symbol] 3.889 3.963 |
| R7 | 55.95 | 30.15 | C_{γ} 27.27; C [symbol] 43.22 | 7.997 | 4.298 | 1.731 1.890 | H_{γ} 1.586, H [symbol] 3.189; H [symbol] 7.271 |
| C8 | 55.45 | ND | — | 8.048 | 4.632 | 3.056 3.436 | — |
| Cit9 | 58.02 | 30.14 | C_{γ} 27.41; C_{δ} 42.32 | 8.150 | 4.166 | 1.709 1.766 | H_{γ} 1.138 1.370; H [symbol] 3.000 |
| iY10 | 56.57 | 38.31 | C_{δ}142.7 133.4; C [symbol] 118.3 | 7.525 | 4.669 | 2.905 3.051 | H_{δ} 7.573 7.101; H [symbol] 6.918 |
| Q11 | 56.09 | 29.30 | C_{γ} 34.05 | 8.152 | 4.161 | 1.928 | H_{γ} 2.097 2.178 |
| C12 | 56.55 | 43.78 | — | 8.287 | 4.660 | 2.986 3.051 | — |
| Y33 | 57.24 | 38.68 | C_{δ} 133.4; C_{δ} 118.2 | 8.076 | 4.599 | 2.905 3.175 | H_{δ} 7.102; H [symbol] 6.795 |

Cit = L-Citralline
iY = L-3-iodo-Tyrosine. [symbol]
indicates data missing or illegible when filed

TABLE 1D-2
Analogue 3

| | C_{α} | C_{β}, | Other C | H_{N} | H_{α}, | H_{β} | Other H |
|---|---|---|---|---|---|---|---|
| E1 | 54.72 | 28.78 | C_{γ} 33.54 | — | 4.193 | 2.165 | H_{γ} 2.406 2.497 |
| G2 | 46.31 | — | — | 8.587 | 3.930 4.020 | — | — |
| C3 | 58.26 | ND | — | 8.421 | 4.900 | 3.009 3.710 | — |
| C4 | ND | 41.66 | — | 8.178 | 4.346 | 3.038 3.215 | — |
| T5 | 62.68 | 68.80 | C_{γ} 21.90 | 8.150 | 4.320 | 4.456 | H_{γ} 1.170 |
| D6 | 50.67 | 38.96 | — | 8.237 | 5.120 | 2.951 3.182 | — |
| P7 | 64.77 | 32.22 | C_{γ} 27.51; C [symbol] 50.93 | — | 4.307 | 1.919 2.391 | H_{γ} 2.068; H_{δ} 3.840 3.948 |
| R8 | 56.36 | 29.54 | C_{γ} 27.37; C [symbol] 43.18 | 7.979 | 4.219 | 1.767 1.891 | H_{γ} 1.579; H_{δ} 3.190; H_{δ} 7.207 |
| C9 | 55.02 | ND | — | 7.887 | 4.642 | 2.915 3.327 | — |
| Cit10 | 57.82 | 30.08 | C_{γ} 28.06; C [symbol] 42.36 | 8.167 | 4.169 | 1.714 | H_{γ} 1.398; H_{δ} 3.190 |
| Y11 | 56.87 | 42.45 | C_{δ} 133.7; C [symbol] 118.3 | 7.428 | 4.629 | 3.034 | H_{δ} 7.053; H [symbol] 6.828 |
| Q12 | 55.59 | 29.15 | C_{γ} 34.09 | 8.031 | 4.308 | 1.920 2.001 | H_{γ} 2.166 2.237 |
| C13 | 56.31 | 42.62 | — | 8.261 | 4.598 | 3.030 3.090 | — |
| Y14 | 57.56 | 38.08 | C_{δ} 133.3; C [symbol] 118.2 | 8.211 | 4.645 | 2.894 3.068 | H_{δ} 7.105; H [symbol] 6.796 |
| K15 | 54.84 | ND | C_{δ} 25.06; C [symbol] 42.68 | 8.174 | 4.398 | 1.697 | H_{γ} 1.877; H_{δ} 1.303 1.449; H [symbol] 3.050 |
| | | | | | | | 3.396; H_{ζ} 7.962 |

Cit = L-Citralline. [symbol]
indicates data missing or illegible when filed

TABLE 1D-3
Analogue 6

| | C_{α} | C_{β}, | Other C | H_{N} | H_{α}, | H_{β} | Other H |
|---|---|---|---|---|---|---|---|
| E1 | 59.65 | 27.97 | C_{γ} 32.13 | — | 4.329 | 2.061 2.491 | H_{γ} 2.399 |
| G2 | 45.91 | — | — | 3.587 | 4.005 4.082 | — | — |
| C3 | 55.84 | ND | — | 8.408 | 4.863 | 3.023 3.632 | — |
| C4 | 57.72 | 39.90 | — | 8.343 | 4.476 | 3.031 3.225 | — |
| T5 | 62.37 | 68.97 | C_{γ} 21.86 | 7.906 | 4.377 | 4.467 | H_{γ} 1.179 |
| D6 | 50.74 | 40.84 | — | 8.134 | 5.125 | 2.829 3.204 | — |
| P7 | 64.73 | 32.23 | C_{γ} 27.48; C_{δ} 51.11 | — | 4.298 | 1.949 2.388 | H_{γ} 2.061; H_{δ} 3.915 3.974 |
| R8 | 56.35 | 29.96 | C_{γ} 27.29; C_{δ} 43.24 | 8.144 | 4.274 | 1.750 1.897 | H_{γ} 1.606; H_{δ} 3.196; H_{δ} 7.330 |
| C9 | 55.44 | ND | — | 7.996 | 4.648 | 3.122 3.503 | — |
| R10 | 57.77 | 29.97 | C_{γ} 26.45; C_{δ} 43.45 | 8.337 | 4.216 | 1.767 | H_{γ} 1.278 1.489; H_{δ} 3.088; H_{δ} 7.066 |
| iY11 | 57.63 | 38.19 | C_{δ}142.6 133.5; C [symbol] 118.1 | 7.650 | 4.610 | 2.900 3.068 | H_{δ} 7.576 7.124; H_{δ} 6.918 |
| Q12 | 56.14 | 29.02 | C_{γ} 34.03 | 8.126 | 4.170 | 1.945 | H_{γ} 2.145 2.202 |
| C13 | 56.60 | 43.34 | — | 8.189 | 4.624 | 2.991 | — |
| Y14 | 56.47 | 38.80 | C_{δ} 133.4; C [symbol] 118.2 | 8.193 | 4.610 | 2.907 3.092 | H_{δ} 7.111; H [symbol] 6.801 |
| K15 | 55.94 | 33.01 | C_{γ} 24.68; C_{δ} 29.06; | 8.206 | 4.251 | 1.711 1.833 | H_{γ} 1.350; H_{δ} 1.643; H [symbol] 2.968; |
| | | | C 42.20 | | | | H [symbol] 7.498 |

iY = L-3-iodo-Tyrosine. [symbol]
indicates data missing or illegible when filed

Structure Calculation. The 3D structures in this research were calculated by deriving inter-proton distance restraints from the intensity of cross-peaks in NOESY (200 ms) and g11-NOESY spectra using CYANA 3.0. Special amino acid libraries (3-iodoTyr, Linked Glu and Lys) were modified on side chain based on natural amino acids. Pseudo-atom corrections were applied to non-stereo-specifically assigned protons. Constraints for the φ, ψ and χ1 backbone dihedral angles were generated from TALOS based on the Hα, Cα, Cβ, and HN chemical shifts. The structures were demonstrated using the program PyMOL and refined with Rosseta. The ensemble of 20 lowest energy structures are superimposed on the backbone atoms (N, O, Cα and Hα) shown as sticks with hydrogens omitted and with calculation statistics presented, as shown in FIGS. 5k to 5m.

### Results and Discussion:

To better understand the effect of side chain cyclization on the whole structure, NMR studies of α-RgIA4 together with analogues 3 and 6 were carried out. A closely correlated secondary Hα chemical shift, particularly on the helical region from Pro6 to Gln11, indicated a high degree of structural similarity between these three molecules, as shown in FIG. 5a-A. Slight variations were observed at the C-terminal region between α-RgIA4 and 3, whereas α-RgIA4 and 6 were more similar. Full three-dimensional solution NMR structures of α-RgIA4, 3 and 6 were then calculated using CYANA3.0. The 20 lowest energy structures from 200 calculated structures were generated with low backbone RMSD. Despite the differences at cyclization-constrained termini and perturbations on side chain linker, both analogue 3 and analogue 6 shared high structural similarities with α-RgIA4 (FIGS. 5a-B and 5a-C), particularly in the Asp5-Pro6-Arg7 “recognition finger” region which is used for receptor binding. Overall, the additional [Glu-Lys] side chain cyclization does not result in structural perturbation to the core of the peptide.

### Example 1-E: Docking Model

### Methods:

Docking Study. Two general classes of analogue 6 conformers were represented in the lowest energy ensembles from the NMR data. Both of these classes were analyzed in Rosetta, however one class (containing 17 of the 20 ensemble structures) generated docked coordinates which successfully reproduced known interactions resolved in the crystal structure of RgIA with α9 subunit (PDB 6HY7). The lowest energy conformer from that class was selected for generating a hypothetical binding model for analogue 6 at α9/α10 nAChR subunit interfaces using Rosetta. Structures of α9 were taken from PDB entries 6HY7 and 4D01, and the homology-modeled coordinates for α10 were taken from previous report. Mutational experiments in vitro have indicated that α-RgIA likely binds the α9(+)/α9(−) and α10(+)/α9(−) interfaces preferentially over the α9(+)/α10(−) interface, so α9(+)/α9(−) and α10(+)/α9(−) were chosen for modeling in Rosetta. Initial docking runs of analogue 6 against receptor subunit interfaces were done using Rosetta Docking Protocol, and allowed for random translational and rotational perturbation of the starting position of analogue 6 at the acetylcholine binding site by 3 Λ and 8°. The Rosetta docking metrics I_sc and rms for the resulting 1000 coordinate files were plotted on a 2D scatter plot, revealing that positioning of analogue 6 in the acetylcholine binding site (aligned to RgIA in PDB file 6HY7) provided the most favorable I_sc scores. There was a significant correlation between I_sc and rms, indicating convergence during the initial docking operation. Further refinement of the interface interactions was done using the [-docking_local_refine flag]. The local refinement results were clustered using I_rms and I_sc to ensure that high-scoring results were not outliers and a final minimization operation was done using Rosetta Relax, as shown in FIGS. 6b and 6c.

### Results and Discussion:

Finally, a docking model of analogue 6 to the receptor based on Rosseta protein-protein docking calculations was generated to help inform SAR efforts. We found that binding interactions revealed by the α-RgIA/α9(+) crystal structure (PDB 6HY7) were predicted by Rosetta to also be present in the case of analogue 6 binding with α9/α10 nAChR. Specifically, analogue 6 residues Asp5 and Arg7 form an intramolecular salt bridge, with the remaining amine on Arg7 hydrogen-bonding to the backbone carbonyl of receptor residue Pro200 on both the α9(+) and α10(+) surfaces, an interaction that is nearly identical to the reported crystal structure, as shown in FIGS. 6a-A and 6a-C. Additionally, Pro6 in analogue 6 forms a CH_{2-π} interaction with Trp151 on loop-B. Further interactions show some variation between the two receptor surfaces studied here, however these apparent differences, which place some potential interacting partners barely beyond the hydrogen-bond cutoff distance, may be overstated by this model as it does not take in to account any potential induced-fit conformational changes in the receptor backbone positions upon ligand binding. Our results indicated additional interactions between Arg9 and the backbone oxygen of Thr152 in the case of the α9(+)/α9(−) interface. Receptor residue Arg59 forms hydrogen bonds with the backbone oxygen of analogue 6 residue Cys3 for both α10(+)/α9(−) and α9(+)/α9(−) and Cys8 for α10(+)/α9(−). Finally, residue Thr4 is predicted to form a hydrogen bond with Asp171, as shown in FIGS. 6a-B and 6a-D. The agreement of these hypothetical models with the reported co-complex of α-RgIA and the human α9(+) surface supports the basis of these predictions. However, given the lack of direct, empirically-derived structural data for the (−) surface with α-RgIA and α9α10 nAChR, structure determination of α-RgIA and related analogues in complex with the full receptor ECD remains a target for future research.

### Example 1-F: Materials and Methods

Materials. All commercially available chemicals were purchased and used directly without further purification. Standard Fmoc protected amino acids were obtained from Protein Technologies Inc. Special protected amino acids including Fmoc-L-Cys(SAcm)-OH, Fmoc-L-Cit-OH, Fmoc-L-3-Iodo-Tyr-OH, Fmoc-beta-Ala-OH, Fmoc-L-Glu(OAll)-OH, Fmoc-L-Lys(NAloc)-OH, Fmoc-L-Asp(OAllyl)=OH, Fmoc-L-Glu(ODmab)-OH, Fmoc-L-Dap(NAloc)-OH, Fmoc-L-Lys(ivDde)-OH and chemicals including HATU, HOBt, PyBOP were purchased from Chemimpex Inc. 2-CTC resin was purchased from ChemPep. EDT, DIEA, DCM, TIPS, DMBA, Pd(PPh₃)₄, iodine, piperidine, ACh, potassium chloride, human serum and BSA were purchased from Sigma Aldrich. DMF, TFA, acetic acid, ACN and ethyl ether were purchased from Fisher Scientific. Oxaliplatin was purchased from MedChem Express.

Animals. All experimental procedures on animals were performed in accordance with the NIH guidelines for the care and use of laboratory animals and were performed under Institutional Animal Care and Use Committees (IACUC) approved protocols by University of Utah. Xenopus laevis frog oocytes used for two electrode voltage clamp experiments were obtained from Xenopus One. Mice for the oxaliplatin experiments were CBA/CaJ inbred strain, available from Jackson Laboratory. All efforts were made to reduce the number of animals used and minimize suffering during procedures.

### Example 2-A: Chemical Synthesis and Characterization of RgIA Methylene Thioacetal Analogues

### Methods:

Conotoxin Analogue Synthesis. Solid-Phase Peptide Synthesis. Linear peptides were synthesized by using automated Fmoc-SPPS chemistry on synthesizer (Syzo I) as previously described using 2-CTC resin.

Cleavage and Purification. Peptides were cleaved off from resin by treatment with a cocktail buffer (TFA:H₂O:TIPS:EDT=95:2:2:1, 3.0 mL/0.1 mmol) for 2.5 h. The obtained Peptide-TFA solution was then filtered via plastic filter and precipitated out into cold ether (40 mL) and cooled at −20° C. for 30 min before pelleted by centrifugation. The crude peptide was washed with cold ether (30 mL) to remove residue TFA and dried in vacuum. The crude product was then purified by RP-HPLC performed on Jupiter 5μ C18 300 Å (250×10 mm) column at 3.0 mL/min with a H₂O/ACN gradient containing 0.1% TFA from 5% to 45% ACN over 40 minutes on an Agilent 1260 HPLC system. The purified fractions containing targeted product were collected and lyophilized by Freeze Dryer (Labconco).

LCMS Analysis. Characterization of peptides was performed by LC/MS on a Phenomenex Gemini C18 3.0 μm (110 Å 150×3 mm) column at 0.4 mL/min flow speed with a H₂O/ACN gradient containing 0.1% formic acid on Agilent 1260 Quadrupole LC/MS system. HPLC purification fractions, purity check for final products, stability assays were also analyzed by LC/MS.

Methylene Thioacetal Formation. The reaction was carried out using a protocol reported by Cramer. The purified linear peptide was dissolved in H₂O and treated with a pre-mixed TCEPHCl (2.0 eq.) and K₂CO₃ (4.0 eq.) in H₂O (19.0 mM). The mixture was gently stirred at room temperature for 2 h. Then Et₃N (10.0 eq. 380 mM in THF) was added to the mixture followed by CH₂I₂ (6.0 eq. 230 mM dissolved in THF). This mixture was allowed to react at room temperature until a complete conversion of linear peptide in about 6 h (Note: Longer reaction time may lead to broad peak on RP-HPLC which is probably caused by amino acid racemization; 5% DMSO could be added in large-scale preparation). I₂ Mediated Disulfide Formation. To the stirred bis-Acm-protected peptide solution in AcOH (aq. 25%, 1.0 mM) was added 12 (10.0 eq.) in AcOH (5.0 mg/mL). The reaction was stirred at room temperature for 10 min and monitored by LC-MS. The excess 12 was quenched by adding 1.0 M solution of ascorbic acid until colorless and the mixture was then purified by RP-HPLC to afford peptides. All fully folded peptides were identified as ≥95% purity by RP-HPLC before NMR analysis and biological assays.

Peptide Characterization. Molecular weights were measured by ESI-MS [M+H]⁺ and [M+2H]²⁺, RgIA-5617: Calc 1705.6 853.3, Found 1705.4 853.2; RgIA-5533: Calc 1705.6 853.3, Found 1705.4 853.4; RgIA-5618, Calc 1719.7 860.4, Found 1719.6 860.4; RgIA-5524, Calc 1874.9 937.9, Found 1874.4, 937.5; RgIA-5573, Calc 1768.8 884.9, Found 1768.5 884.9.

### Results and Discussion:

The chemical synthesis of RgIA analogues was achieved by using 9-fluorenylmethyloxycarbonyl (Fmoc) solid-phase peptide synthesis (SPPS) on 2-chlorotrityl chloride (2-CTC) resin followed by a two-operation and regioselective intramolecular bond formation reactions, as shown in FIGS. 7a-A, 7a-B, 7a-C, 7a-D. The correct scaffold folding is Cys^{I}-Cys^{III}, Cys^{II}-Cys^{IV} or its corresponding methylene thioacetal replacement with the same connectivity. Bonds were explicitly formed in an order of 1) methylene thioacetal formation on free Cys after trityl (Trt) removal through cleavage, 2) disulfide bond formation via in situ oxidative acetamidomethyl (Acm) deprotection-coupling process, and 3) repeating methylene thioacetal formation to generate the bis-methylene thioacetal replaced analogue. In detail, after cleavage of the assembled peptide chain from 2-CTC resin, the Trt protections were removed and the target methylene thioacetal bond was formed by treatment with diiodomethane in the presence of tris(2-carboxyethyl)phosphine hydrochloride (TCEP·HCl), potassium carbonate and trimethylamine (Et₃N). This operation conversion can be conducted in as large as 300 mg scale in one batch, which allows large preparation of target peptides for further studies. The second disulfide bridge was formed after Acm deprotection by the treatment of excess iodine in 25% aqueous acetic acid (AcOH) to yield fully folded peptides. RgIA and RgIA4 were synthesized as described herein. All peptides were purified as ≥95% purity indicated by RP-HPLC and final products were analyzed by ESI-MS before NMR studies and biological assays, as shown in FIGS. 7B-a and 7B-b, Table 2A-1, and LC chromatography, as shown in FIGS. 7c to 7g.

TABLE 2A-1

| | Molecular Weight (Da)ª | | | | |
|---|---|---|---|---|---|
| Peptide | Calculated | | Observed | | Purity |
| No. | [M + H]⁺ | [M + 2H]²⁺ | [M + H]⁺ | [M + 2H]²⁺ | (%)^{b} |
| RgIA- | 1705.6 | 853.3 | 1705.4 | 853.2 | >99 |
| 5617 | | | | | |
| RgIA- | 1705.6 | 853.3 | 1705.4 | 853.4 | 98 |
| 5533 | | | | | |
| RgIA- | 1719.7 | 860.4 | 1719.6 | 860.4 | 98 |
| 5618 | | | | | |
| RgIA- | 1874.9 | 937.9 | 1874.4 | 937.5 | 98 |
| 5524 | | | | | |
| RgIA- | 1768.8 | 884.9 | 1768.5 | 884.9 | 96 |
| 5573 | | | | | |

^{a}Determined by ESI mass spectrometry.
^{b}Determined by RP-HPLC.

### Example 2-B: In Vitro Biological Evaluation of RgIA Methylene Thioacetal Analogues

### Methods:

Two-Electrode Voltage-Clamp (TEVC) Recording. We followed a method as follows. Briefly, Xenopus laevis oocytes were used to express cloned rat or human nAChR subtypes heterologously. Recordings were performed 1-3 days postinjection. Oocytes were voltage-clamped at a membrane potential of −70 mV in a 30 μL oocyte chamber which was gravity perfused at a flowrate of 2-4 mL/min with ND-96 buffer containing 0.1 mg/mL BSA. A 1 s ACh (100 μM for all subtypes, with the exception of 200 μM for α7 and 10 μM for the muscle subtype) pulse per minute was applied to establish a baseline. Then ND96 solution containing the various concentrations of test peptides was switched and the ACh responses were measured until a steady state reached. All recordings were generated at room temperature and repeated as 3-6 independent experiments. Data analyses were performed with GraphPad Prism software and values including the resulting IC₅₀ were calculated using a nonlinear regression sigmoidal dose-response.

### Results and Discussion:

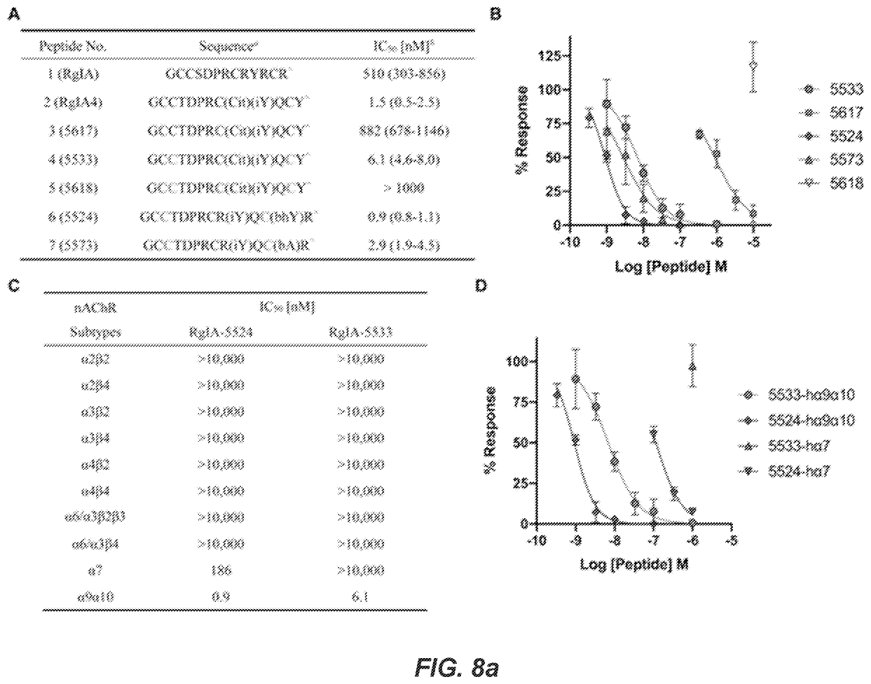

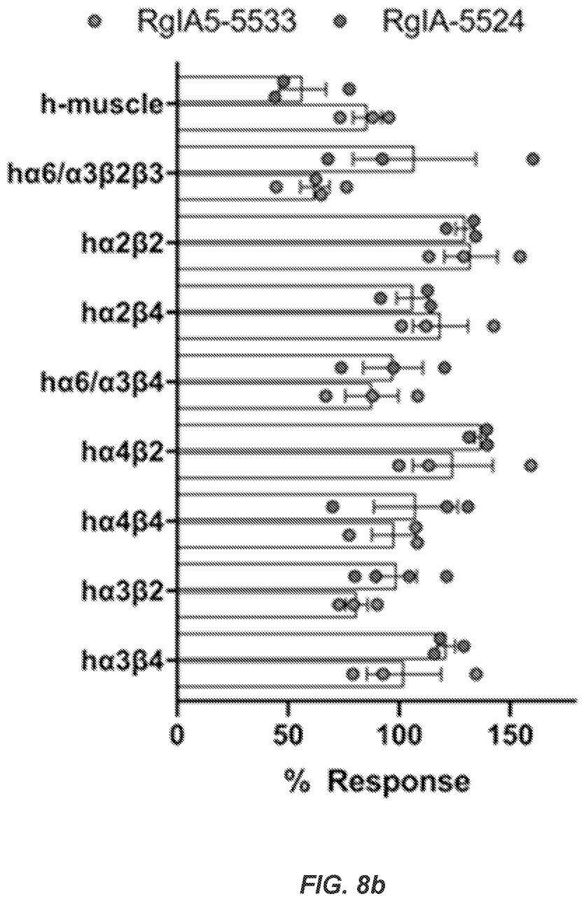

The bioactivities of all synthesized analogues were tested by two-electrode-voltage-clamp (TEVC) electrophysiology on human α9α10 nAChRs heterologously expressed in Xenopus laevis oocytes. To determine the compatibility of methylene thioacetal as a disulfide surrogate in the RgIA series, a suite of analogues, as depicted in FIG. 8a-A with different methylene thioacetal replacements were synthesized and tested. IC₅₀ values determined by concentration-response analysis, as shown in FIG. 8a-B. By comparison, native RgIA inhibits ACh-evoked currents mediated by human α9α10 nAChRs with an IC₅₀ value of 510 nM due to the low affinity for the human receptor.

Differential effects were produced when methylene thioacetal was introduced to the sequence of a modified RgIA analogue, RgIA4. Specifically, RgIA-5533, which had the loop II [Cys^{II}-Cys^{IV}] disulfide exchanged for methylene thioacetal, had low nanomolar potency (IC₅₀=6.1 nM). In contrast, as for analogue RgIA-5617, with the methylene thioacetal functionality moved to loop I [Cys^{I}-Cys^{III}], there was a tremendous decrease in potency (IC₅₀=880 nM). Activity was further eliminated when both disulfides were replaced in RgIA-5618 (IC₅₀>10 μM). These data show that the loop II disulfide [Cys^{II}-Cys^{IV}] in RgIA is amenable to modification with methylene thioacetal whereas substitution at the other position [Cys^{I}-Cys^{III}] abolishes activity for human α9α10 nAChRs. The results are consistent with the pioneering research of dicarba modified analogues of RgIA in which both the trans cis isomers of [Cys^{II}-Cys^{IV}]-dicarba RgIA maintained a much reduced activity for the α9α10 nAChR whereas the [Cys^{I}-Cys^{III}]-dicarba analogues were completely inactive. Similarly, the effect of the [Cys^{I}-Cys^{III}] disulfide on the structure and activity was also demonstrated in another α-4/3-CTxs ImI by analyzing disulfide-deficient analogues. The RgIA-5533 was modified with mutants based on RgIA5 as well as a non-canonical amino acid, β-homotyrosine (bhTyr), to afford the potent analogue RgIA-5524 with an IC₅₀ value of 0.9 nM. Single residue mutation of bhTyr with β-Alanine (bAla) yielded an analogue, RgIA-5573, which was less potent (IC₅₀=2.9 nM) indicating the effect of a phenolic moiety at residue position 13.

The subtype selectivity of RgIA-5533 and RgIA-5524 was investigated. Two-electrode-voltage-clamp (TEVC) electrophysiology showed that both analogues failed to inhibit a wide range of nAChR subtypes at 10 μM (IC₅₀>10 μM) including α1β1δε, α2β2, α2β4, α3β2, α3β4 α4β2, α4β4, α6/α3β2β3 and α6/α3β4, as shown in FIG. 8a-C and FIG. 8b. Concentration-response analysis indicated that both RgIA-5533 and RgIA-5524 exhibited nanomolar IC₅₀s on α7 nAChR yet still were greater than 200-fold selective for hα9α10 nAChRs, as shown in FIG. 8a-D. Testing RgIA-5524 via a competition binding assay using [¹²⁵1]α-bungarotoxin (α-Btx) as radioligand demonstrated that RgIA-5524 produced 410% inhibition at 10 μM level, consistent with its low potency on the hα7 nAChR subtype, as shown in FIGS. 8a-C and 8a-D.

### Example 2-C: In Vivo Pain-Relieving Efficacy of RgIA-5524

### Methods:

In Vivo Antinociceptive Activity Evaluation. Neuropathic Pain Model. All experimental procedures on animals were performed in accordance with the NIH guidelines for the care and use of laboratory animals and were performed under Institutional Animal Care and Use Committees' (IACUC) approved protocols at the University of Utah. All efforts were made to minimize suffering. Male CBA/CaJ mice (2-3 months old) were injected with oxaliplatin. For the chronic administration group, oxaliplatin was administered i.p., at 3.5 mg/kg 5 days per week over a period of 21 days. For acute administration groups either 5.0 mg/kg oxaliplatin or 10.0 mg/kg oxaliplatin was given as a single dose. 0.9% Saline was used as vehicle control. Cold Plate Test. A cold plate test was performed using a hot/cold plate (IITC Life Science). Mice were allowed to acclimatize to the testing chamber until investigative behavior subsided. Then the plate temperature lowered from room temperature using a linear ramp (10° C./min). The time and temperature of the first pain-related behavior (lifting and licking of the hind paw) were recorded. Raters were blind to drug and mouse genotype. Statistical evaluations of the data were performed by one-way analysis of variance (ANOVA) followed by Dunnett's multiple comparison test. All results are expressed as means±SEM (n=8-12). P values were *P<0.05, **P<0.01, and ***P<0.001 for significant difference.

### Results and Discussion:

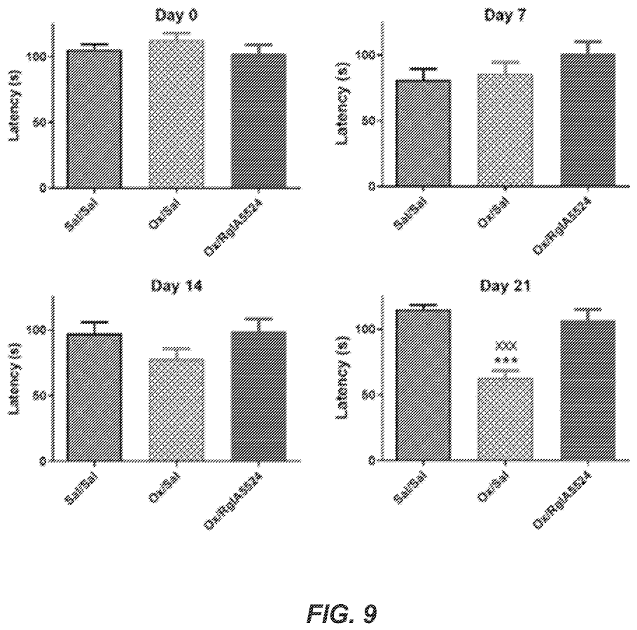

Chemotherapy-induced neuropathic pain is a major dose-limiting side effect of platin-based drugs. Currently, the pathophysiology of oxaliplatin-induced neuropathic pain remains poorly investigated and there are no approved drugs for the prevention of this dose-limiting adverse outcome. The in vivo analgesic activity of RgIA-5524 was assessed using a model of oxaliplatin-induced peripheral neuropathic pain in mice, as shown in FIG. 9. Cold-allodynia is a disabling side effect of oxaliplatin. The magnitude and time course of this side effect is dose-dependent. Repeated daily injections of RgIA-5524 prevented the development of neuropathic pain caused by chemotherapy. Oxaliplatin (i.p. 3.5 mg/kg, 5 days each week) created substantial cold allodynia by day 21 of treatment as indicated by significantly reduced paw withdrawal latency on the cold plate. In contrast, oxaliplatin-treated mice that received 40 μg/kg of RgIA-5524 did not develop allodynia.

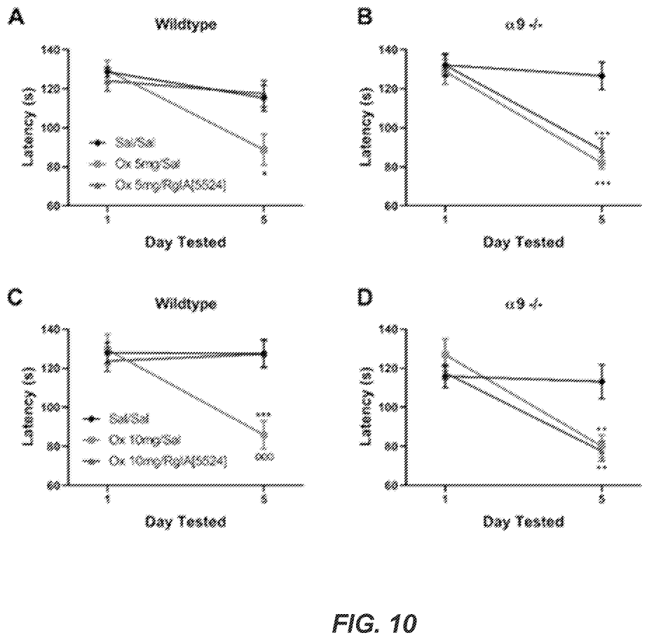

We next performed the single-injection oxaliplatin treatment study in both wild-type and α9 KO mice. The results show that RgIA-5524 was effective in reversing the acute cold allodynia 5 days after oxaliplatin treatment (s.c. 5.0 mg/kg and 10.0 mg/kg) as indicated by significant differences between the WT group of Sal/Sal vs Ox/Sal and Ox/RgIA-5524 vs Ox/Sal, as shown in FIGS. 10A and 10C. However, this effect did not occur in the α9 KO mice group, where no significance was observed between the α9 KO group of Ox/RgIA-5524 vs Ox/Sal, as shown in FIGS. 10B and 10D. This KO experiment demonstrated that the blockade of α9α10 nAChR by RgIA-5524 enabled the prevention or attenuation of chemotherapy-induced neuropathic pain.

### Example 2-D: In Vitro Pharmacological, Toxicity and Metabolism Assays of RgIA-5524

### Methods:

In Vitro Pharmacology Assays. Generally, RgIA-5524 was initially tested in quadruplicate at a default concentration of 10 μM in assays. A secondary assay was performed to determine a concentration-response curve when RgIA-5524 blocked higher than 50% of the radioligand binding.

Competition Binding and Enzyme Assays. Cell membrane homogenates were incubated with the radioligand in the absence or presence of RgIA-5524. Nonspecific binding was determined in the presence of a specific agonist or antagonist at the target. Following incubation, the samples were filtered rapidly under vacuum through glass fiber filters presoaked in a buffer and rinsed several times with an ice-cold buffer using a 48-sample or 96-sample cell harvester. The filters were then counted for radioactivity in a scintillation counter using a scintillation cocktail.

hERG K⁺ Channel Inhibition Assay. Automated whole cell patch-clamp (Qpatch 16) on human hERG transfected CHO-K1 cells was used to record outward potassium currents. After whole cell configuration was achieved at 22° C., the cell was held at −80 mV. A 50 ms pulse to −40 mV was delivered to measure the leaking current. Then the cell is depolarized to +20 mV for 2 s followed by a 1 s pulse to −40 mV to reveal the hERG K⁺ tail current. This paradigm is delivered every 5 s to monitor current amplitude. The exocellular solution is applied first followed by RgIA-5524 solution sequentially on the same cell. E-4031 was tested as reference ligand.

Functional Study of RgIA-5524 at GABA_{B1b} Receptors. Cells were suspended in DMEM buffer and then distributed in microplates. Fluo4 NW mixed with probenicid in HBSS buffer complemented with 20 mM Hepes (pH 7.4) is then added into each well and equilibrated with the cells for 60 min at 37° C. then 15 min at 22° C. Thereafter, the assay plates are positioned in a microplate reader, the reference agonist or antagonist (3-APMPA) at one concentration (stimulated control) or various concentrations (EC₅₀ or IC₅₀ determination) were added and the measurements of changes in fluorescence intensity which varies proportionally to the free cytosolic Ca²⁺ ion concentration.

CYP Enzyme Isoform Inhibition Assays. RgIA-5524 is pre-incubated with NADPH-generating system in PBS 7.4 for 5 min in a 37° C. dry incubator. The reaction is initiated by adding a mixture of a CYP enzyme isoform, a substrate, and BSA. The fluorescence in each well is read before and after the incubation period. The percent inhibition is calculated by subtracting the percent of control.

### Results and Discussion:

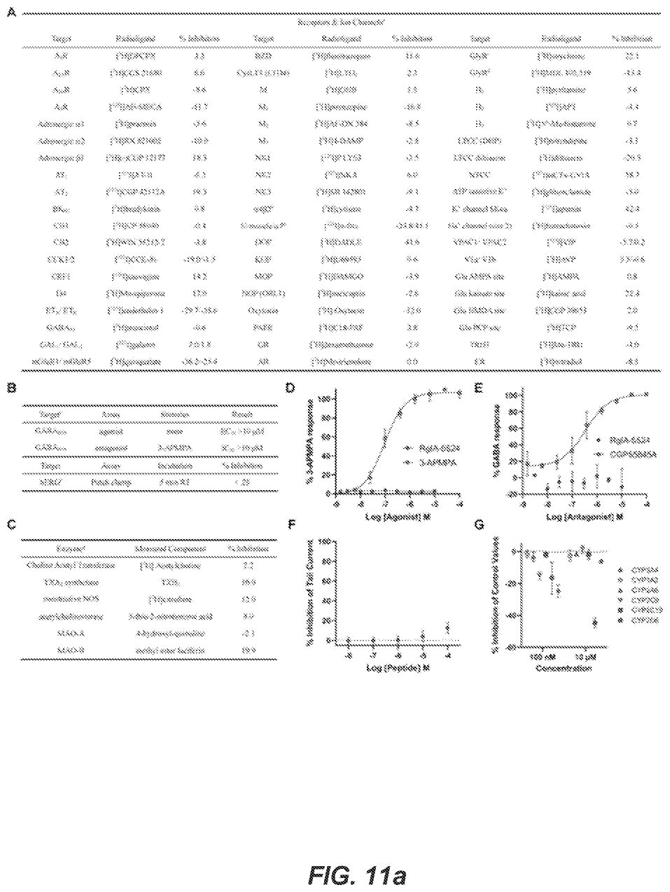

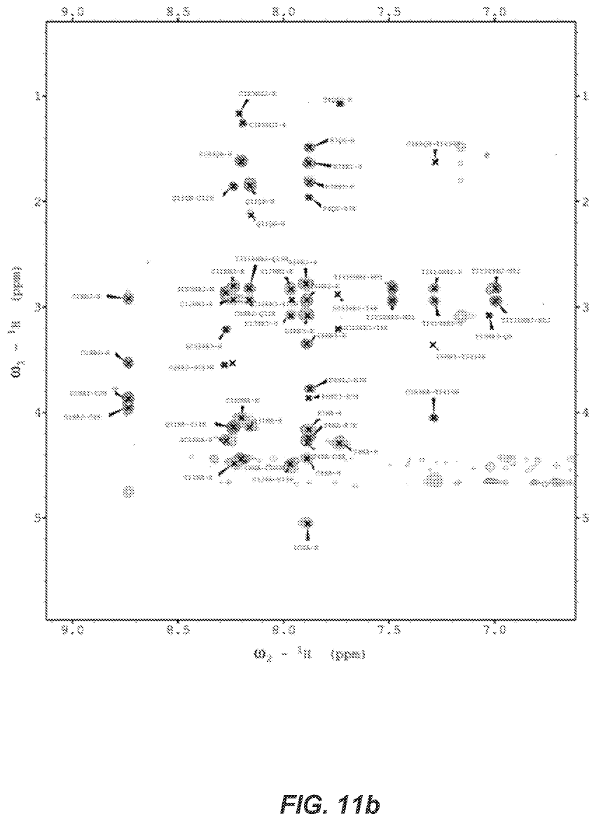

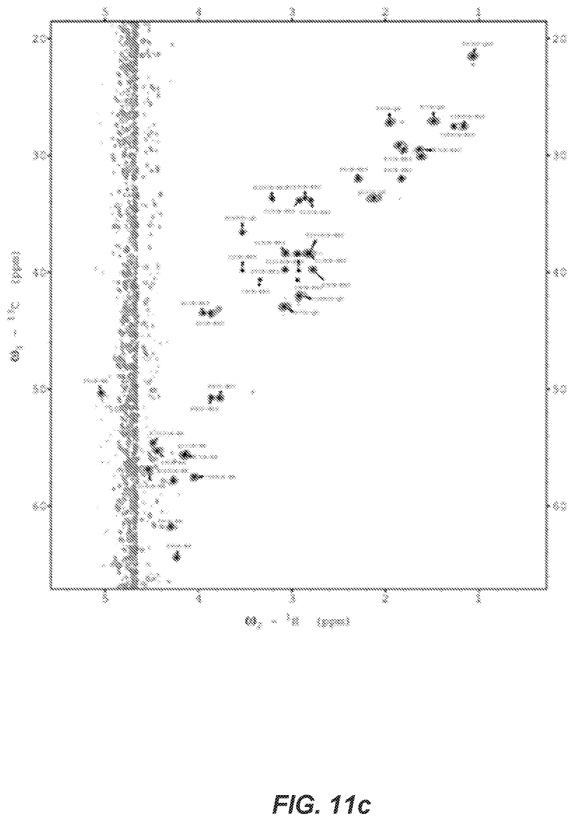

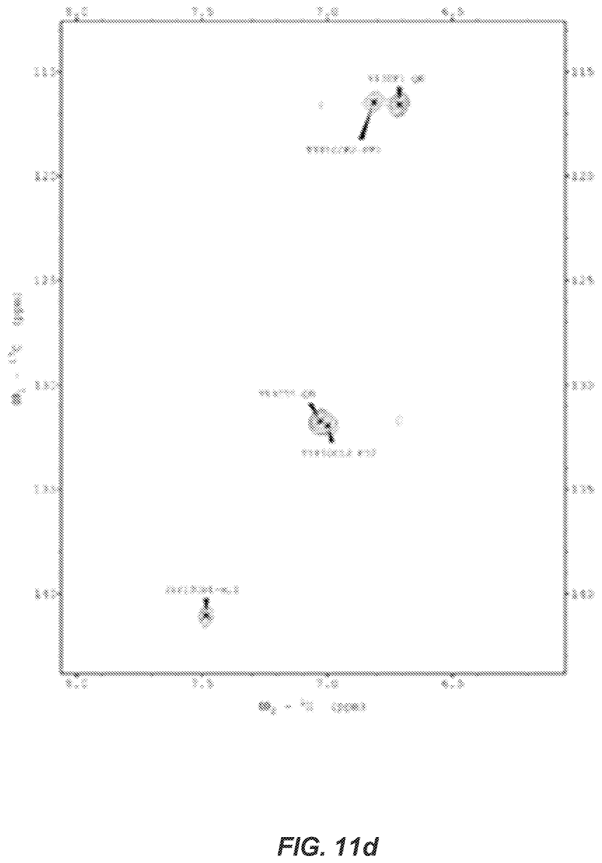

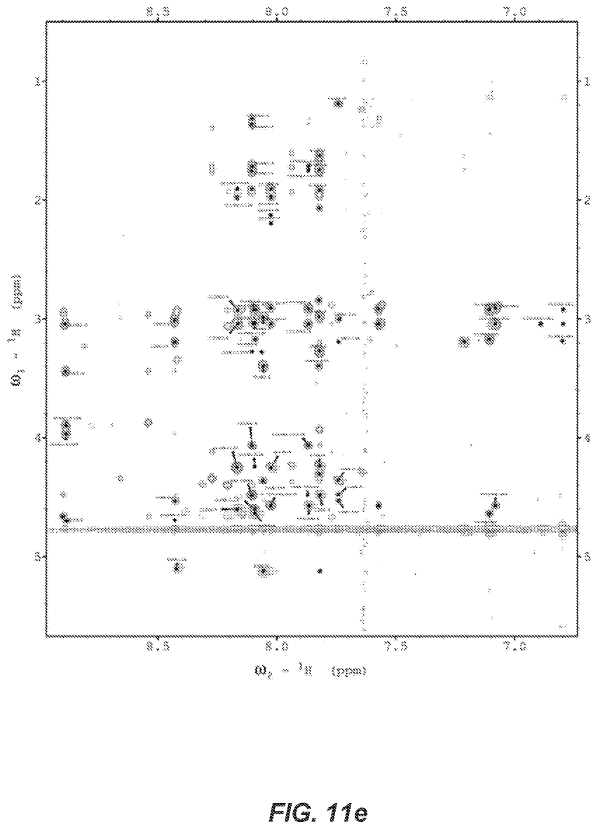

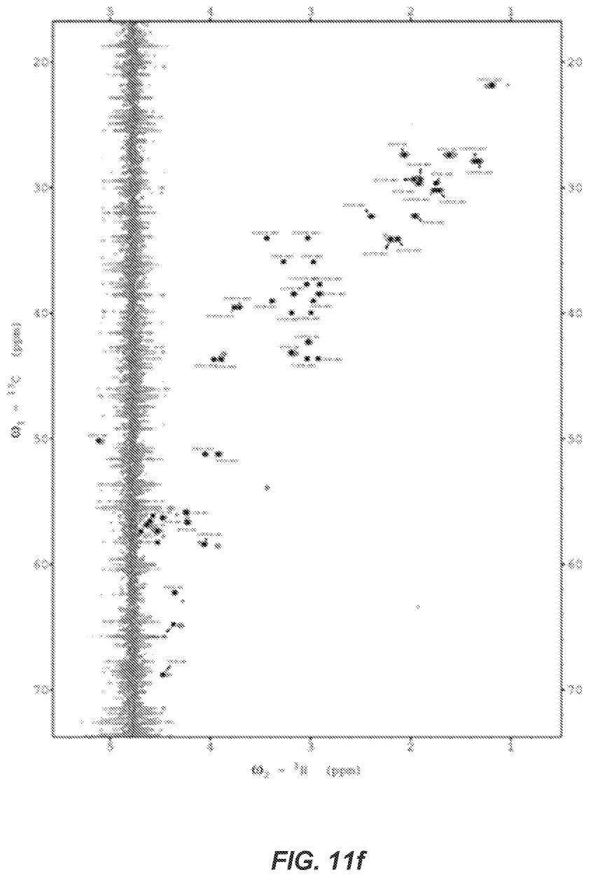

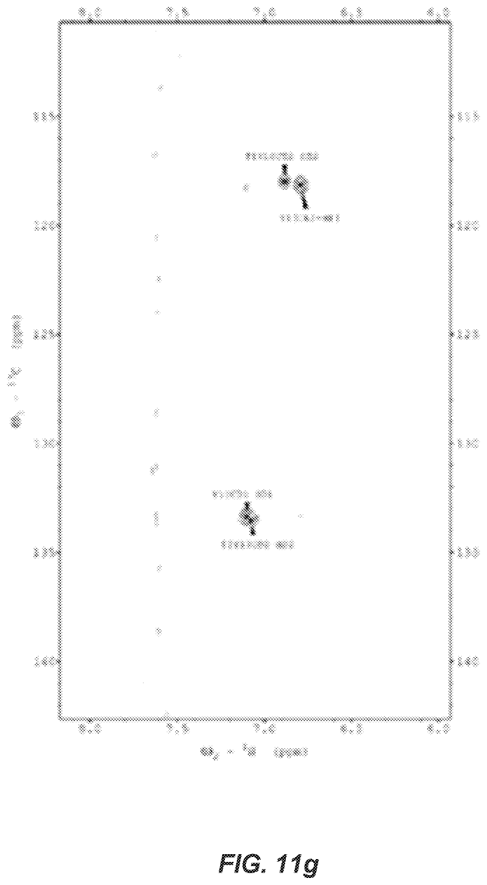

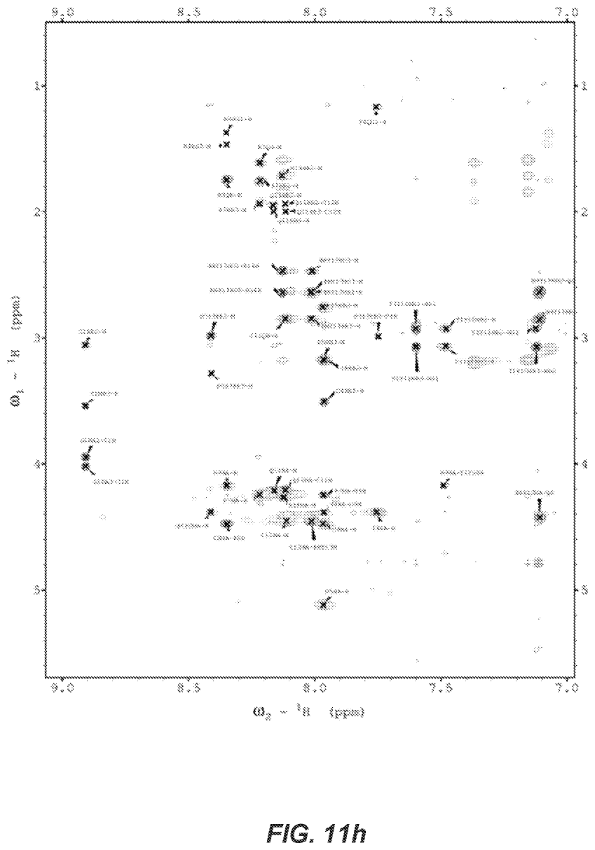

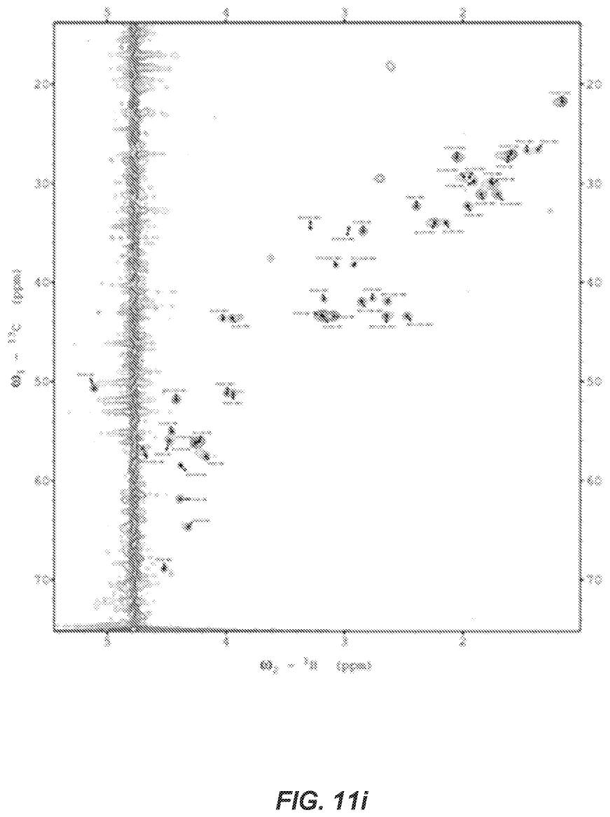

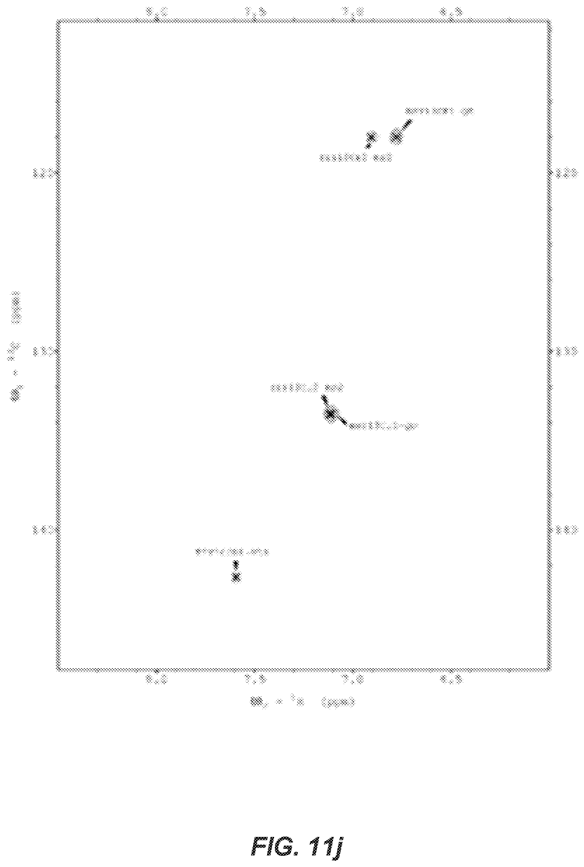

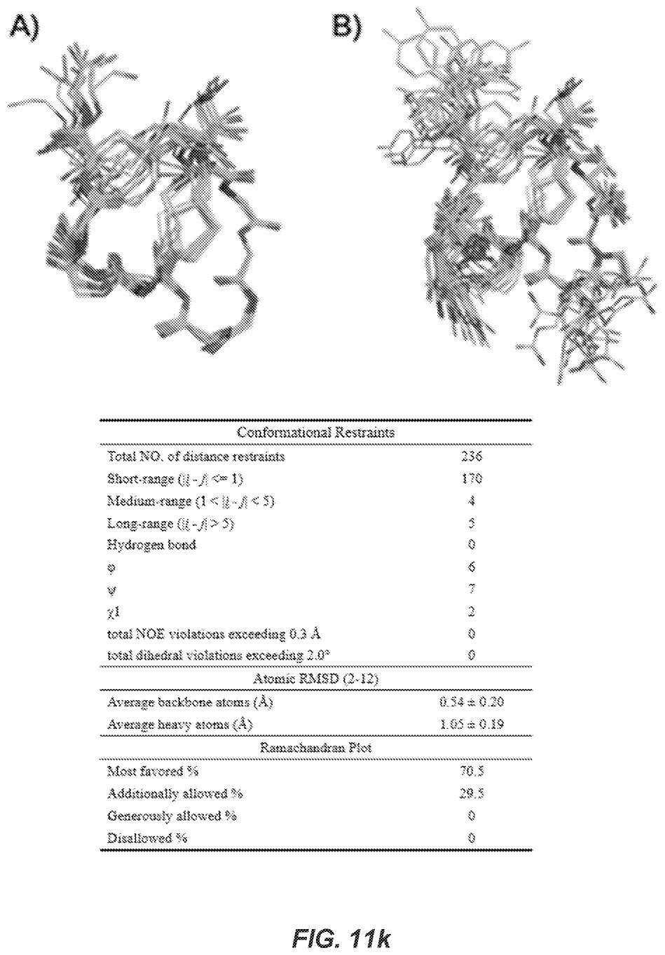

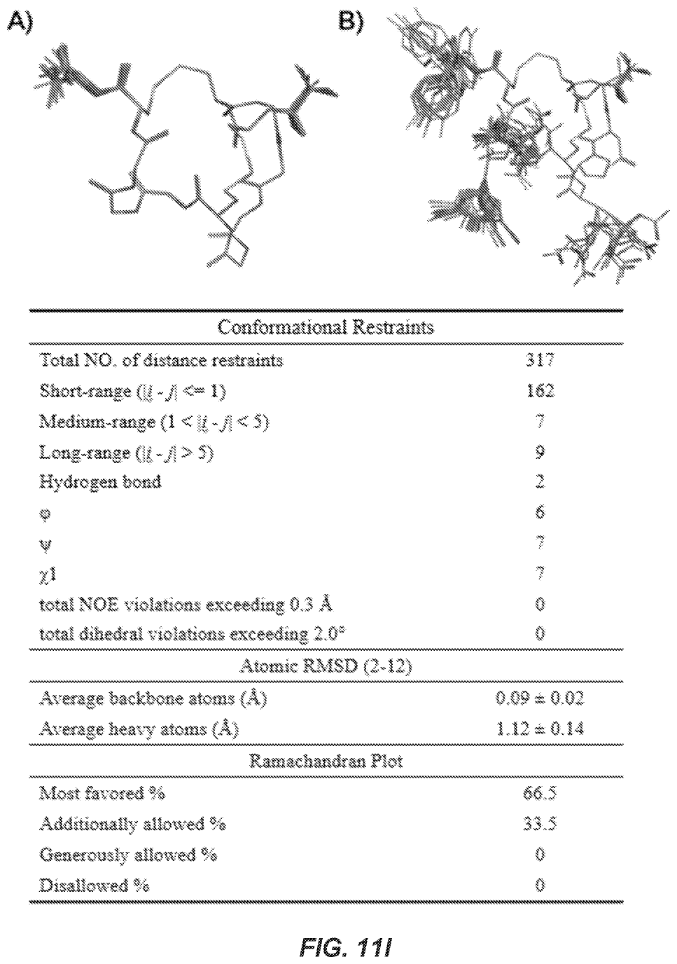

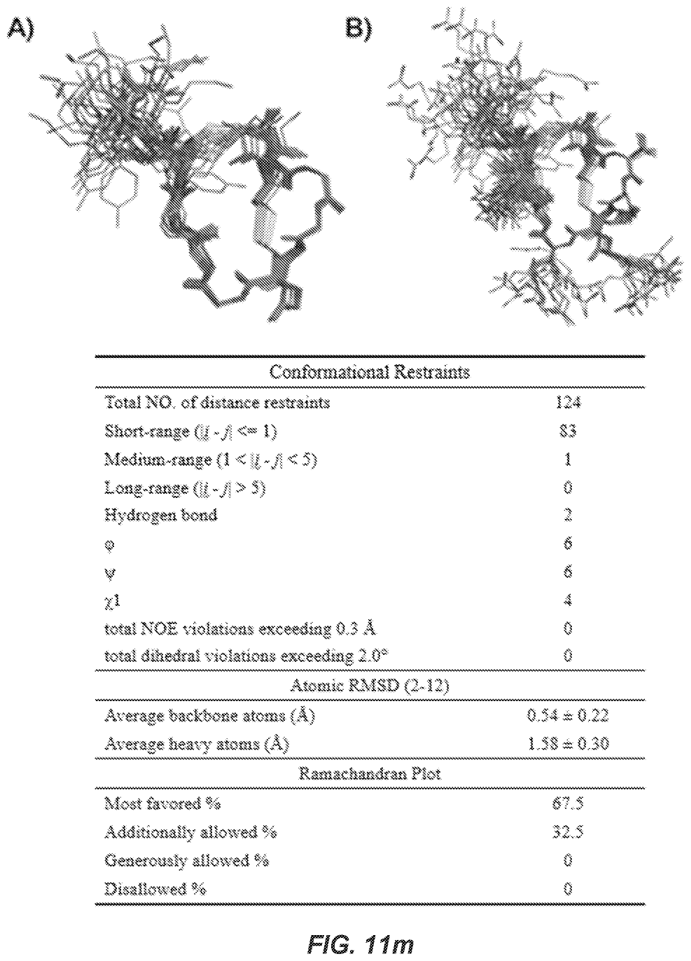

We further demonstrated that the most potent analogue RgIA-5524 was a promising non-opioid analgesic candidate via a broad suite of in vitro pharmacology assays. First, we tested RgIA-5524 on a wide range of various pain-associated receptors and ion channels. As summarized in FIG. 11a-A, at 10 μM level, RgIA-5524 showed low or no activity (<50% inhibition) on these potential targets including the opioid receptors, NMDAR, BZD, OCT receptors, and various voltage-gated ion channels (Na⁺, K⁺ & Ca²⁺). Testing RgIA-5524 on N-type Ca²⁺ channel showed low potency with 58.4% inhibition at 10 μM, whereas further concentration-response analysis demonstrated micromolar affinity, too low to account for the analgesic activity. Utilizing a cellular dielectric spectroscopy assay, we also demonstrated that RgIA-5524 failed to display any concentration-dependent agonist or antagonist effects at GABA_{B1b} receptors, which has been a postulated mechanism for RgIA analgesia, as shown in FIGS. 11a-B, 11a-D, and 11a-E. Taken together with the above in vivo α9 KO mice study, the results firmly demonstrate that antagonizing α9-containing nAChRs is the dominant mechanism of the observed RgIA-5524 analgesic effects.

Drug-induced cardiotoxicity has become one of the major reasons leading to drug withdrawal in the past decades, which is closely related to the blockade of the human ether-a-go-go-related gene (hERG) K⁺ channel. No evidence for cardiovascular liability was indicated from an automated-whole cell patch-clamp assay in which RgIA-5524 caused <25% inhibition at a high concentration of 100 μM, as shown in FIGS. 11a-B and 11a-F. Meanwhile, RgIA-5524 is inactive in a set of enzyme and uptake assays including acetylcholinesterase and MAO, which can be used in several neurodegenerative disorders, as shown in FIG. 11a-C. Finally, we assessed the potential of RgIA-5524 to influence drug-drug interactions; no inhibition was observed against a wide panel of CYP enzyme isoforms at 10 μM, as shown in FIG. 11a-G.

### Example 2-E: NMR Spectroscopy and Structural Analysis

### Methods:

Structural Analysis. NMR Spectroscopy. Peptide samples (prepared at concentration of 2.0 mM dissolved in a 10% D20 containing buffer pH 3.5 with 20 mM Na₂HPO₄, 50 mM NaCl, 50 μM NaN₃, and 0.1 mM EDTA, uncorrected for isotope effects) were recorded at 298 K on an Inova 600 MHz spectrometer. Secondary structure determination was achieved using TOCSY (80 ms), NOESY (200 ms), g11-NOESY, gCOSY, and HSQC. Excitation sculpting schemes were used for water suppression. Spectra were analyzed using NMRPipe and SPARKY. Molecular representations were prepared using PyMOL program. Overlay of the amide regions of TOCSY (blue) and NOESY (red) with HSQC aliphatic region and aromatic region. Assignments were made using SPARKY. See e.g., FIGS. 11b to 11j, and Tables 2E-1, 2E-2, and 2E-3 (with Scs=L-S-methylene-Cysteine; Cit=L-Citrulline, Tiy=L-3-iodo-Tyrosine).

TABLE 2E-1
RgIA-5533

| Res | C_{α} | C_{β}, | Other C | H_{N} | H_{α}, | H_{β} | Other H |
|---|---|---|---|---|---|---|---|
| G1 | 43.49 | — | — | — | 3.871, | — | — |
| | | | | | 3.949 | | |
| C2 | ND | 39.86 | — | 8.736 | 4.752 | 2.921, 3.535 | — |
| Scs3 | 57.85 | 33.67 | C_{δ} 36.59 | 8.273 | 4.269 | 2.869, 3.212 | — |
| T4 | 61.76 | 68.51 | C_{γ} 21.51 | 7.738 | 4.295 | 4.426 | H_{γ} 1.067 |
| D5 | 50.34 | 39.77 | — | 7.890 | 5.050 | 2.783, 3.076 | — |
| P6 | 64.39 | 32.01 | C_{γ} 27.20; C_{δ} 50.75 | — | 4.232 | 1.834, 2.291 | H_{γ} 1.958; H_{δ} 3.7 [symbol] |
| | | | | | | | 3.861 |
| R7 | 55.64 | 29.49 | C_{γ} 27.07; C_{δ} 42.95 | 7.879 | 4.157 | 1.638, 1.807 | H_{γ} 1.483; H_{δ} 3.086; |
| | | | | | | | 7.159 |
| C8 | 55.29 | 40.65 | — | 7.890 | 4.434 | 2.940, 3.347 | — |
| Cit9 | 57.52 | ND | C_{γ} 27.50; C_{δ} 42.02 | 8.199 | 4.046 | 1.617 | H_{γ} 1.162; 1.259; |
| | | | | | | | 2.921 |
| Tiy10 | ND | 38.39 | C_{δ} 141.1; 132.0; | 7.285 | 4.640 | 2.821 | H_{δ} 7.486; 6.994; |
| | | | C_{ε} 116.5 | | | | 6.811 |
| Q11 | 55.64 | ND | C_{γ} 33.65 | 8.160 | 4.137 | 1.843 | H_{γ} 2.124; H_{ε} 7.661 |
| C12 | 54.59 | 33.86 | — | 8.238 | 4.484 | 2.808, 2.924 | — |
| Y13 | 56.84 | 38.36 | C_{δ} 131.7; C_{ε} 116.6 | 7.965 | 4.530 | 2.834, 3.075 | H_{δ} 7.023; H_{ε} 6.714 |

[symbol]

indicates data missing or illegible when filed

TABLE 2E-2
RgIA-5617

| Res | C_{α} | C_{β}, | Other C | H_{N} | H_{α}, | H_{β} | Other H |
|---|---|---|---|---|---|---|---|
| G1 | 43.68 | — | — | — | 3.898, | — | — |
| | | | | | 3.966 | | |
| Scs2 | 57.38 | 34.03 | C_{δ} 3.707 3.769 | 8.887 | 4.691 | 3.036, 3.443 | — |
| C3 | 57.35 | 39.97 | — | 8.429 | 4.530 | 3.002, 3.193 | — |
| T4 | 64.79 | 68.79 | C_{γ} 21.86 | 7.740 | 4.362 | 4.477 | H_{γ} 1.191 |
| D5 | 50.17 | 39.02 | — | 8.058 | 5.118 | 2.986, 3.393 | — |
| P6 | 62.28 | 32.26 | C_{γ} 27.38; C_{δ} 51.23 | — | 4.307 | 1.967, 2.394 | H_{γ} 2.070; H_{δ} 3.92 [symbol] |
| | | | | | | | 4.057 |
| R7 | 56.66 | 29.65 | C_{γ} 27.41; C_{δ} 43.16 | 7.822 | 4.235 | 1.744, 1.916 | H_{γ} 1.623; H_{δ} 3.192 |
| C8 | 56.29 | 35.92 | ND | 7.821 | 4.481 | 2.978, 3.274 | — |
| Cit9 | 58.43 | 30.21 | C_{γ} 27.89; C_{δ} 42.31 | 8.404 | 4.064 | 1.710, 1.756 | H_{γ} 1.317; 1.370; |
| | | | | | | | 3.028 |
| Tiy10 | 56.13 | 37.72 | C_{δ} 133.6; C_{ε} 118.0 | 7.868 | 4.571 | 2.911, 3.042 | H_{δ} 7.215; 7.079; |
| | | | | | | | 6.886 |
| Q11 | 55.87 | 29.32 | C_{γ} 34.10 | 8.026 | 4.249 | 1.904, 1.972 | H_{γ} 2.123; 2.198 |
| C12 | 56.56 | 43.62 | ND | 8.164 | 4.602 | 2.931, 3.038 | — |
| Y13 | 56.86 | 38.48 | C_{δ} 133.4; C_{ε} 118.2 | 8.093 | 4.638 | 2.921, 3.172 | H_{δ} 7.106; H_{ε} 6.797 |

[symbol]

indicates data missing or illegible when filed

TABLE 2E-3
RgIA-5524

| Res | C_{α} | C_{β}, | Other C | H_{N} | H_{α}, | H_{β} | Other H |
|---|---|---|---|---|---|---|---|
| G1 | 43.64 | — | — | — | 3.946, | — | — |
| | | | | | 4.018 | | |
| C2 | ND | ND | — | 8.908 | 4.803 | 3.056, 3.534 | — |
| Scs3 | 58.43 | 34.51 | — | 8.412 | 4.387 | 2.985, 3.283 | H_{δ} 3.630 |
| T4 | 61.85 | 68.90 | C_{γ} 21.76 | 7.753 | 4.387 | 4.518 | H_{γ} 1.166 |
| D5 | 50.69 | 41.67 | — | 7.965 | 5.116 | 2.760, 3.173 | — |
| P6 | 64.67 | 32.31 | C_{γ} 27.38; C_{δ} 51.19 | — | 4.320 | 1.962, 2.394 | H_{γ} 2.050; H_{δ} 3. [symbol] |
| | | | | | | | 3.995 |
| R7 | 56.14 | 29.80 | C_{γ} 27.34 | 8.216 | 4.245 | 1.759, 1.919 | H_{γ} 1.611; H_{δ} 3.197 |
| | | | | | | | 7.368 |
| C8 | 55.93 | ND | ND | 7.964 | 4.480 | 3.180, 3.508 | — |
| R9 | 57.52 | 30.05 | C_{γ} 26.44; C_{δ} 43.41 | 8.347 | 4.171 | 1.748 | H_{γ} 1.374; 1.463; |
| | | | | | | | 3.094 |
| Tiy10 | 56.73 | 38.24 | C_{δ} 142.6; 133.5; C_{ε} 118.0 | 7.482 | 4.706 | 2.927, 3.069 | H_{δ} 7.599; 7.123; |
| | | | | | | | 6.912 |
| Q11 | 56.03 | 29.36 | C_{γ} 33.95 | 8.163 | 4.213 | 1.947, 1.998 | H_{γ} 2.147; 2.231 |
| C12 | 55.04 | 34.82 | ND | 8.114 | 4.456 | 2.849 | — |
| Bhy13 | 51.84 | 41.92 | C_{δ} 133.5; C_{ε} 118.0; | 8.011 | 4.422 | 2.637, 2.855 | H_{○} 2.474; 2.644; |
| | | | C_{○} 43.38 | | | | 7.110; H_{ε} 6.780 |
| R14 | 56.20 | 31.09 | C_{γ} 27.04; C_{δ} 43.41 | 8.127 | 4.257 | 1.713, 1.839 | H_{γ} 1.593; H_{δ} 3.177 |
| | | | | | | | 7.155 |

[symbol]

indicates data missing or illegible when filed

Structure Calculations. Three dimensional structures were calculated from the two-dimensional spectra using CYANA 3.0 with backbone dihedral angle constrains predicted by TALOS program. Non-canonical amino acids (L-citrulline, L-3-iodo-tyrosine, L-S-methylene-cysteine and L-β-homotyrosine) were built based on their corresponding natural amino acids using CYANA 3.0 as previously described. The 20 lowest-energy ensembles out of total 200 calculated structures were chosen for further analysis of the Cα distance measurement. The ensemble of 20 lowest energy structures are superimposed on the backbone atoms (N, O, Cα and Hα) shown as sticks with hydrogens omitted and with calculation statistics presented, as shown in FIGS. 11j to 11m.

### Results and Discussion:

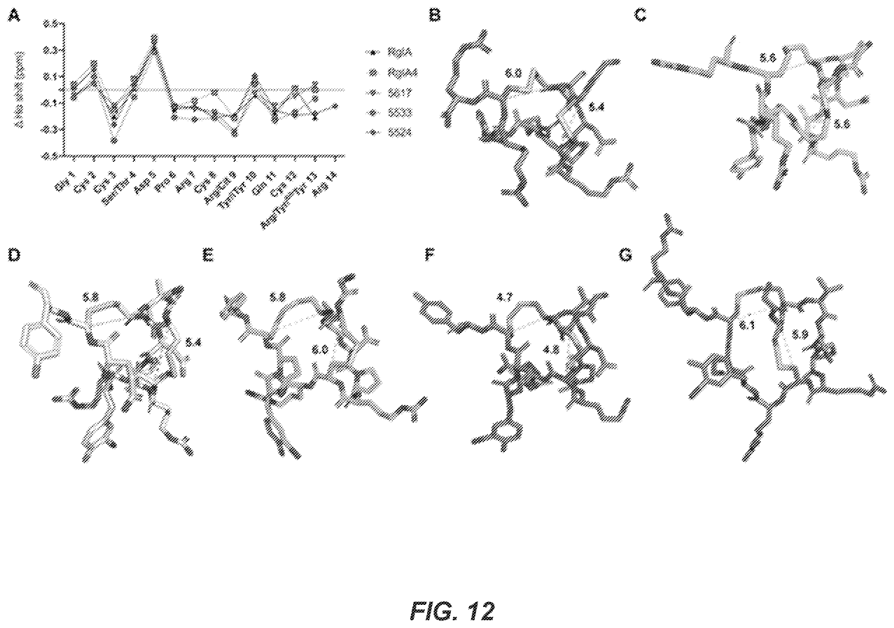

NMR studies of the modified analogues were performed to compare and contrast the structural features. Analogues including RgIA-5533, RgIA-5617, and RgIA-5524 were analyzed by homo-nuclear 2D ¹H-NMR spectroscopy including TOCSY, NOESY, COSY and HSQC. Assignments on all residues except for N-terminal Gly¹ primary amine were achieved. In all molecules studied, Pro⁴ was identified as trans conformation by strong NOEs observed from Asp⁵ Hα to Pro⁶ Hδ. Hα secondary-shift analysis was used to assess any changes in the secondary structure elements. In general, secondary Hα shift confirmed that RgIA-5533, 5524, and 5617 all maintained the globular conformations. Subtle changes were observed in the residues including Asp⁵-Pro⁶-Arg⁷ and Arg/Cit⁹-Tyr/iTyr¹⁰-Gln/Arg¹¹ segments. No obvious differences among the potent analogue RgIA-5533, RgIA-5524, or inactive RgIA-5617 compared with that of native disulfides bonded RgIA and RgIA4 were observed from Hα secondary chemical shifts. Slight deviations existed at C-termini of individual peptides primarily due to terminal flexibility, as shown in FIG. 12A.

Three-dimensional NMR solution structures of these analogues were calculated using CYANA 3.0 with atom distance and dihedral angle restraints generated from g11-NOESY and NOESY (200 ms) spectra. Backbone dihedral angle restraints including φ, ψ, and χ1 were predicted by the TALOS program based on the Hα, Cα, Cβ and amide hydrogen (HN) chemical shifts. The 20 lowest energy state ensembles were obtained with low RMSD. Together with previously reported structures of RgIA (NMR solution structure PDB 2JUQ and co-crystal extract PDB 6HY7) and RgIA4, the “closest to mean” energy states were chosen to represent each peptide and shown as FIGS. 12B, 12C, 12D, 12E, 12F, and 12G with average Cα distance of cysteine pairs measured by PyMOL program. All methylene thioacetal modified peptides maintained globular conformations closely resembling that of RgIA and RgIA4. The most significant difference between RgIA-5617 and the potent analogues (RgIA4, RgIA-5533, and 5524) were the Cα distances of cysteine pairs. Both the Cα distance of the two cysteine pairs in RgIA-5617 are apparently shortened (average of 4.8 and 4.7 Å in cysteine loop I and loop II, receptively) compared with the other molecules (average of 5.4-6.1 Å). One potential reason for the potency loss of analogue RgIA-5617 and RgIA-5618 is that the insertion of CH₂ group in loop I disulfide [Cys^{I}-Cys^{III}] forced a conformation “shrink” in these loop I modified analogues to accommodate the dihedral and torsional angle changes, which reduced their binding affinities. It has also been proposed by MD stimulation that the loop I disulfide in RgIA analogues might provide stacking interaction towards the receptor by directly contacting with the C-loop disulfide of the α9(+) surface. Therefore, methylene thioacetal replacement at this loop could cause potency loss by interfering with this binding site, which could be another contributing factor despite their minor secondary structure perturbations.

### Example 2-F: In Vitro Stability Assays

### Methods:

Stability Assays. Testing peptides were dissolved in PBS 7.4 at concentration of 1.0 mg/mL for stock solution and were further diluted with either human serum (AB type, Sigma-Aldrich), or PBS 7.4 containing reduced glutathione (10 equiv.) to a final testing peptide concentration of 0.1 mg/mL. Then the diluted solutions were incubated at 37° C. and portions of the mixture was taken up at predetermined time points for RP-HPLC analysis. Serum protein were removed by denaturation with addition of equal volume of ACN, cooled on ice for 10 min and followed by centrifugation at 13,000 g for 10 min. The supernatant was collected and analyzed by RP-HPLC. The stability at each time point was calculated as the area of the treated peptide peaks (220 nm) on RP-HPLC as percentage of the area of the 0 h treated peptides. Each experiment was performed in triplicate. Data were analyzed by student t (unpaired) test. P values were **P<0.01, ***P<0.001 for significant difference at each time point.

### Results and Discussion:

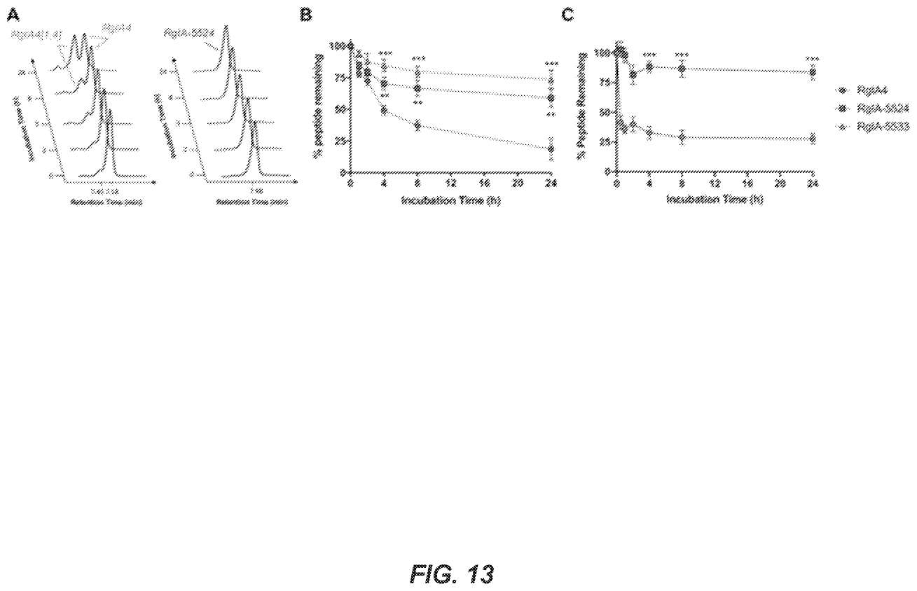

Generally, disulfide-folded peptides and proteins have rigid structures which result in relatively enhanced stability against proteases. However, free reducing thiols in human serum can interfere with the disulfide connectivity of cysteine-rich peptides by scrambling and thus lead to enzymatic degradation and potency loss. To determine how methylene thioacetal influenced the metabolic stability of RgIA4, in vitro human serum stability assay of RgIA-5544 and RgIA-5533 were performed. Peptides (0.1 mg/mL in 90% human serum AB type) were continuously incubated in human serum at 37° C. for 24 h and the amount of remaining peptide was determined by RP-HPLC at time point 0, 1, 2, 4, 8, and 24 h post-incubation. As shown in FIG. 13A, RgIA4 scrambled rapidly into its isomer RgIA4[1,4] and ended up with less than 25% of the globular RgIA4, which is consistent with our previous observations. RgIA-5533 was significantly more stable than RgIA4 where above 70% of the peptide was intact even after 24 h incubation. RgIA-5524 was slightly less stable compared with RgIA-5533 possibly due to its higher arginine-rich sequence which can be cleaved by trypsin, as shown in FIG. 13B. Also, complete disulfide scrambling inhibition was achieved when methylene thioacetal was introduced. We also assessed the stability of RgIA-5524 against RgIA4 in the presence of reduced glutathione (GSH) at physiological pH. Similar to human serum degradation results, single methylene thioacetal replacement in RgIA-5524 was able to largely suppress disulfide scrambling, as shown in FIG. 13C. Overall, RgIA-5524 exhibited significantly enhanced stability which makes it a more attractive and promising candidate for further developments.

### Example 2-G: Materials and Methods

Chemicals. All chemicals were purchased and used directly without further purification. Fmoc-protected amino acids and reagents were purchased from Chemimpex, Thermal Fischer, and Sigma Aldrich. Oocytes. Xenopus laevis frog oocytes used for two-electrode voltage clamp experiments were purchased from Xenopus One. Mice. CBA/CaJ inbred strain mice (2-3 weeks, male) used for in vivo assays were obtained from Jackson Laboratory.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Reference throughout this specification to “an example” means that a particular feature, structure, or characteristic described in connection with the example is included in at least one embodiment of the present disclosure. Thus, appearances of the phrases “in an example” in various places throughout this specification are not necessarily all referring to the same embodiment.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

The foregoing detailed description describes the disclosure with reference to specific exemplary embodiments. However, it will be appreciated that various modifications and changes can be made without departing from the scope of the present disclosure as set forth in the appended claims. The detailed description and accompanying drawings are to be regarded as merely illustrative, rather than as restrictive, and all such modifications or changes, if any, are intended to fall within the scope of the present disclosure as described and set forth herein.

1. An α-RgIA4 peptide analog comprising:
a recognition finger region configured to bind to an α9α10 nicotinic acetylcholine receptor; and
a side chain bonding configuration that protects an inter-cysteine sulfur linkage,
wherein the analog has a binding affinity for the α9α10 nicotinic acetylcholine receptor that is at least 2.5% of a binding affinity of an α-RgIA4 peptide.

2. (canceled)

3. The α-RgIA4 peptide analog as in claim 1, wherein the binding affinity for the α9α10 nicotinic acetylcholine receptor is:
at least 5% of the binding affinity of the α-RgIA4 peptide, or
at least 7.5% of the binding affinity of the α-RgIA4 peptide, or
at least 15% of the binding affinity of the α-RgIA4 peptide, or
at least 25% of the binding affinity of the α-RgIA4 peptide, or
at least 40% of the binding affinity of the α-RgIA4 peptide, or
at least 50% of the binding affinity of the α-RgIA4 peptide, or
at least 80% of the binding affinity of the α-RgIA4 peptide, or
substantially equal to the binding affinity of the α-RgIA4 peptide, or greater than the binding affinity of an α-RgIA4 peptide.

4. The α-RgIA4 peptide analog as in claim 1, wherein the protected inter-cysteine sulfur linkage provides an increase in potency compared to a potency of an α-RgIA4 peptide.

5. The α-RgIA4 peptide analog as in claim 1, wherein the analog provides an α9α10 nicotinic acetylcholine receptor IC₅₀ value that is:
substantially equal to an α9α10 nicotinic acetylcholine receptor IC₅₀ value of the α-RgIA4 peptide, or
no greater than 2.0× the α9α10 nicotinic acetylcholine receptor IC₅₀ value of the α-RgIA4 peptide, or
no greater than 3.0× the α9α10 nicotinic acetylcholine receptor IC₅₀ value of the α-RgIA4 peptide, or
no greater than 5.0× the α9α10 nicotinic acetylcholine receptor IC₅₀ value of the α-RgIA4 peptide, or
no greater than 15.0× the α9α10 nicotinic acetylcholine receptor IC₅₀ value of the α-RgIA4 peptide, or
no greater than 25.0× the α9α10 nicotinic acetylcholine receptor IC₅₀ value of the α-RgIA4 peptide.

6. The α-RgIA4 peptide analog as in claim 1, wherein the protected inter-cysteine sulfur linkage reduces one or more of disulfide bridge scrambling, disulfide bridge degradation, or a combination thereof as compared to an α-RgIA4 peptide, or α-RgIA4 peptide analog without a protected inter-cysteine sulfur linkage.

7. The α-RgIA4 peptide analog of claim 1, wherein the side chain bonding configuration comprises a one or more of a methylene thioacetal, an N-terminal amino acid side chain that is cyclized to a C-terminal amino acid side chain with a lactam bridge, or a combination thereof.

8. The α-RgIA4 peptide analog of claim 7, wherein the side chain bonding configuration is a methylene thioacetal comprising an inter-cysteine linkage between C^{II} and C^{IV}.

9. The α-RgIA4 peptide analog of claim 7, wherein the side chain bonding configuration is an N-terminal amino acid side chain that is cyclized to a C-terminal amino acid side chain with a lactam bridge.

10. The α-RgIA4 peptide analog of claim 9, wherein the N-terminal amino acid is selected from the group consisting of glutamic acid and aspartic acid.

11. The α-RgIA4 peptide analog of claim 9, wherein the C-terminal amino acid is selected from the group consisting of lysine and L-2,3-diaminopropionic acid.

12. The α-RgIA4 peptide analog of claim 9, wherein the N-terminal amino acid is glutamic acid and the C-terminal amino acid is lysine.

13. The α-RgIA4 peptide analog as in claim 1, wherein the protected inter-cysteine sulfur linkage provides a stability for the α-RgIA4 peptide analog in human serum that is greater than the stability of an α-RgIA4 peptide in human serum, wherein the stability in the human serum is measured by the amount remaining after incubation of 0.1 mg/mL of the α-RgIA4 peptide analog or the α-RgIA4 peptide in 90% human serum AB type and incubated at 37° C. for at least one of 1, 2, 4, 8, 24, 48, or 72 hours.

14. The α-RgIA4 peptide analog of claim 13, wherein the stability in human serum of the α-RgIA4 peptide analog is at least one or more of 10%, 20%, 40%, 60%, 80%, 100%, 200%, 300%, 400%, 500%, or 1000% greater than the stability of the α-RgIA4 peptide in human serum.

15. The α-RgIA4 peptide analog as in claim 1, wherein the protected inter-cysteine sulfur linkage provides a stability for the α-RgIA4 peptide analog in reduced glutathione that is greater than the stability of an α-RgIA4 peptide in reduced glutathione, wherein the stability in the reduced glutathione is measured by the amount remaining after incubation of 0.1 mg/mL of the α-RgIA4 peptide analog or the α-RgIA4 peptide in 10 equivalents of reduced glutathione in phosphate buffered saline (PBS) having a pH of 7.4 and incubated at 37° C. for at least one of 1, 2, 4, 8, 24, 48, or 72 hours.

16. The α-RgIA4 peptide analog of claim 15, wherein the stability in the reduced glutathione of the α-RgIA4 peptide analog is at least one or more of 10%, 20%, 40%, 60%, 80%, 100%, 200%, 300%, 400%, 500%, or 1000% greater than the stability of the α-RgIA4 peptide in the reduced glutathione.

17. The α-RgIA4 peptide analog as in claim 1, wherein the protected inter-cysteine sulfur linkage provides an α9α10 nicotinic acetylcholine receptor selectivity that is substantially equal to the α9α10 nicotinic acetylcholine receptor selectivity of an α-RgIA4 peptide.

18. The α-RgIA4 peptide analog as in claim 1, wherein the protected inter-cysteine sulfur linkage provides a α9α10 nicotinic acetylcholine receptor selectivity that is at least one or more of 5×, 10×, 20×, 50×, 100×, or 200× more selective for the α9α10 nicotinic acetylcholine receptor compared to a selectivity of a different nicotinic acetylcholine receptor (nAChR) subtype.

19. The α-RgIA4 peptide analog of claim 18, wherein the different nAChR subtype is selected from the group consisting of: α1β1δε, α2β2, α2β4, α3β2, α3β4α4β2, α4β4, α6/β3β2β3 and α6/α3β4.

20. The α-RgIA4 peptide analog as in claim 1, wherein the protected inter-cysteine sulfur linkage provides a safety profile that is substantially equal to or greater than the safety profile of an α-RgIA4 peptide, wherein the safety profile is measured by one or more of:
the analog present in a concentration of 100 μM inhibits less than 25% of the human ether-a-go-go-related gene (hERG) K⁺ channel as measured from an automated-whole cell patch-clamp assay,
the analog present in a concentration of 100 μM has inhibitory activity of less than about 20% as measured by a monoamine oxidase (MAO) assay, or
the analog present in a concentration of 10 μM has inhibitory activity of less than 20% as measured in a CYP assay.

21. The α-RgIA4 peptide analog as in claim 1, wherein the protected inter-cysteine linkage is one or more of an inter-cysteine linkage between C^{I} and Cu^{III}, C^{II} and C^{IV}, or a combination thereof.

22. The α-RgIA4 peptide analog of claim 21, wherein the structure is globular.

23-85. (canceled)
